(19) United States
(12) Patent Application Publication
(10) Pub. No.: US 2016/0078168 A1
(43) Pub. Date: Mar. 17, 2016

(54) FUSION TRANSCRIPT DETECTION METHODS AND FUSION TRANSCRIPTS IDENTIFIED THEREBY

(71) Applicant: SPLICINGCODES.COM, Palmetto Bay, FL (US)

(72) Inventors: Degen ZHUO, Palmetto Bay, FL (US); Xiaoyan YANG, Zionsville, IN (US)

(73) Assignee: SPLICINGCODES.COM, Palmetto Bay, FL (US)

(21) Appl. No.: 14/792,613

(22) Filed: Jul. 7, 2015

Related U.S. Application Data
(63) Continuation-in-part of application No. 13/372,180, filed on Feb. 13, 2012.

(51) Int. Cl. G06F 19/22; C12Q 1/68

(52) U.S. Cl. CPC G06F 19/22; C12Q 1/6886; C12Q 2600/156

(57) ABSTRACT

This present disclosure generally relates to compositions and methods for cancer diagnosis, research and therapy, including but not limited to, cancer markers. In particular, the present disclosure provides a computerized method for detecting fusion transcripts from RNA-seq data and provides the fusion transcripts identified thereby in human cancers. Compositions and methods for identifying the fusion transcripts are also provided.

### CROSS-REFERENCE TO RELATED APPLICATIONS

The present application is a continuation-in-part of U.S. patent application Ser. No. 13/372,180, filed Feb. 13, 2012, the contents of which are hereby incorporated by reference in its entirety.

### REFERENCE TO SEQUENCE LISTING SUBMITTED ELECTRONICALLY

The content of the electronically submitted sequence listing, file name Human_Cancer_Fusion_Transcripts20150705.txt, size 176,469,241 bytes; and date of creation Jul. 5, 2015, filed herewith, is incorporated herein by reference in its entirety.

### BACKGROUND OF THE INVENTION

Cancer is one of the leading causes of deaths in the world and a class of heterogeneous complex diseases with multiple genes in diverse pathways involved in its initiation, uncontrolled growth, invasion, and metastasis. One of the cancer hallmarks is genetic instabilities that can result in chromosomal translocation, insertion, duplication, deletion, and inversion. These genetic alternations often cause fusion genes, which in turn are transcribed into fusion mRNAs or fusion transcripts (Mitelman, et al. 2007). Numerous methods have been developed to characterize the cancer genomic aberrations. Introduction of molecular cytogenetic technologies such as chromosomal fluorescence in situ hybridization (FISH) and multicolor FISH into the repertoire of clinical testing and genetic investigation has led to an explosion of information about chromosomal aberrations in cancers, which has greatly improved our understanding of the prevalence and variety of these genomic rearrangements. Comparative genomic hybridization (CGH) and—array CGH are developed to detect chromosomal aberration and copy-number variations in cancers. Applications of these technologies in clinical and genetic investigations have accumulated an abundance of information about chromosomal aberrations, which is stored in NCI's Cancer Chromosomes database (Mitelman, et al. 2015).

Next-generation sequencing of transcriptomes (RNA-seq) is one of the most recent technological advances and provides one of the most important tools to unbiasedly profile gene expression and to uncover the novel splice sites. However, RNA-Seq faces several bioinformatics challenges from developing efficient methods to storing, retrieving and processing large amounts of RNA-Seq data, which disproportionally accumulate highly expressed mRNA sequences. Existence of spliceosomal introns in gene sequences, especially in the mammalian genes makes analyses of these short sequences more problematic and computationally expensive. To overcome these challenges, a number of softwares have been developed to profile gene expression and to identify novel alternatively-spliced splice sites and fusion transcripts. The software to be able to detect fusion transcripts include TopHat-Fusion, SOAPfusion, SnowShoes-FTD, ShortFuse, BreakFusion, ChimeraScan, Comrad, FusionAnalyser, deFuse, FusionMap, FusionHunter, FusionSeq, R-SAP, Trans-ABySS and Trinity.

These technological advances have led to the identification of multiple novel fusion transcripts (Klijn, et al. 2015, Robinson, et al. 2011, Sakarya, et al. 2012). More recently, transcriptome sequencing and RNA-seq have been used to identify the fusion genes (Maher, et al. 2009, Zhao, et al. 2009). Using paired-end RNA sequencing, Maher et al. has identified 12 novel chimeric transcripts of fusion genes in 4 cancer cell line (Maher, et al. 2009). Edgren et al. have applied paired-end RNA-seq to identify 24 novel and 3 previously known fusion genes in breast cancer cells (Edgren, et al. 2011). The software improvement has led to the identification of more fusion transcripts (Kim and Salzberg 2011). Recently, Sakarya et al. have used next-generation sequencing to analyze MCF-7 breast cancers and have identified 40 novel fusion genes (Sakarya, et al. 2012). More recently, Klijn et al have performed comprehensive RNA-seq analysis of 675 human cancer cell lines and have identified 2,200 unique pairs of fusion genes, 1,435 of which had been previously not found (Klijn, et al. 2015). Many of these chimeric transcripts have shown to have multiple isoforms (Robinson, et al. 2011). The read-though fusion transcripts have been shown to be associated with breast cancer (Varley, et al. 2014).

However, current approaches are inefficient to analyze large RNA-seq datasets. Majority of them often are very slow and require large memories and powerful computation systems. They are effective to uncover highly-expressed fusion transcripts and may be unable to discover lowly-expressed fusion transcripts. Because some algorithms used may be unintentional to remove some fusion transcripts from considerations. A large amounts of RNA-seq datasets have been accumulated in ENCODE (ENCODE 2015), ENA (ENA 2014) and NCBI (NCBI 2014). However, the numbers of fusion transcripts identified so far remain small considering cancer extreme heterogeneities and complexities.

### SUMMARY OF THE INVENTION

This application generally relates to a method for identifying fusion transcripts in cancers, and more specifically to a computerized method for identifying fusion transcripts from RNA sequencing data obtained from cancer cells. The application also relates to sequences of fusion transcripts identified by the above method.

Previously, the applicant had disclosed a method of identifying exons and introns from predetermined genome data including nucleotide sequence data, predetermined 5′ and 3′ splicing junction data, and exon and intron data (U.S. Pat. No. 8,185,323). The contents of the above patent are hereby incorporated by reference in its entirety.

The applicant had observed that recently-gained human spliceosomal introns had identical 5′ and 3′ splice sites (Zhuo, et al. 2007). Based on this finding, the applicant had found that both 5′ exonic sequences (E5) immediately upstream of introns and 3′ intronic sequences (13) were dynamically conserved and appears rather reminiscent of self-splicing group II ribozymes and of constraints imposed by base pairing between intronic-binding sites (IBSs) and exonic-binding sites (EBSs) (Zhuo, et al. 2012). Therefore, the applicant has proposed that both E5 and I3 sequences constitute splicing codes, which are deciphered by splicer proteins/RNAs via specific base-pairing (Zhuo, et al. 2012). This splicing code model suggested that a yet-to-be characterized splicer proteins/RNA would decode identical sequences in all pre-mRNAs in conjugation with U snRNAs and spliceosomes, regardless whether the E5 and I3 sequences are in the one molecule or two different molecules.

Based on this splicing code model, the applicant has developed a simple, accurate and fast computation system to analyze RNA-seq data for the discovery of fusion transcripts, and has identified a large number of novel fusion transcripts, some of which can be used for early detection and prognosis of cancer.

Disclosed herein includes a method of detecting alternatively spliced transcripts or fusion transcripts in at least one RNA sequence obtained from biochemical analysis of a biological sample from a species or from a database, comprising the steps of:

(a) providing a computer for data identification, aligning, and comparison purposes, wherein the computer has access to predetermined genome data of said species, comprising data of predetermined genomic nucleotide sequences, predetermined splicing junctions, predetermined exons, predetermined introns, and annotated genes;

(b) generating a splicing code table using the predetermined genome data, the splicing code table comprising ordered E5 keys, I5 keys, E3 keys and I3 keys, wherein the E5 keys, the I5 keys, the E3 keys and the I3 keys are subsequences of predetermined 5′ exonic (E5), 5′ intronic (I5), 3′ exonic (E3), and 3′ intronic (I3) splicing sequences for each of the predetermined splicing junctions respectively;

(c) aligning the at least one RNA sequence with each of the E5 keys and each of the E3 keys in the splicing code table; and

(d) determining that the at least one RNA sequence is an alternatively spliced transcript if: the at least one RNA sequence contains a first subsequence substantially identical to an E5 key of a first splicing junction and a second subsequence substantially identical to an E3 key of a second splicing junction of the same gene; or the at least RNA sequence contains a subsequence substantially identical to an E5 key of an annotated gene, but an immediate downstream sequence of said subsequence is mapped to an intron region of the same annotated gene; or the at least one RNA sequence contains a subsequence substantially identical to an E3 key of a splicing junction, but an immediate upstream sequence of said subsequence is mapped to an intron region of the same annotated gene; or determining that the at least one RNA sequence is a fusion transcript if: the at least one RNA sequence contains a subsequence substantially identical to an E5 key of a first annotated gene, and an immediate downstream sequence of said subsequence is substantially identical to an E3 key of a second annotated gene; or the at least RNA sequence contains a subsequence substantially identical to an E5 key of a first annotated gene, and an immediate downstream sequence of said subsequence is mapped to a second annotated gene; or the at least one RNA sequence contains a subsequence substantially identical to an E3 key of a first annotated gene, and an immediate upstream sequence of said subsequence is mapped to a second annotated gene.

In some embodiments of the method, the E5 keys, the I5 keys, the E3 keys and the I3 keys in the splicing code table in step (b) have a length of about 20-50 bp.

In some embodiments of the method, the at least one RNA sequence is obtained from a biochemical analysis such as RT-PCR followed by direct sequencing, RNA sequencing, and transcriptome sequencing (whole-genome RNA sequencing). In some embodiments, the at least one RNA sequence may be retrieved from an online database in which a set of predetermined RNA sequences are deposited.

In some embodiments, the method for detecting alternatively spliced transcripts or fusion transcripts in RNA sequences may further comprising a quality control step between step (b) and step c), wherein the quality control step comprises removing reads from the at least one RNA sequence, wherein the reads have substantially same sequences as at least one of mitochondrial gene sequences, mitochondrial ribosomal RNA sequences, ribosomal RNA sequences, poly (A) sequences, GC-repetitive sequences, AT-rich sequences, and simple and contaminant sequence reads.

This method of analyzing RNA sequences for detecting alternatively spliced transcripts or fusion transcripts as disclosed above can be applied to any eukaryotic organism where RNA splicing occurs. Examples of such applications in mammals includes human, mouse or rat. The at least one RNA sequences can be obtained from a biological sample, such as a cell line, a tissue, or a cell-free plasma sample.

Disclosed herein also includes a method of utilizing knowledge of predetermined fusion transcripts to identify one or more such fusion transcripts from a transcriptome RNA sequencing data obtained from a biological sample, and to then quantitatively determine the expression level of the fusion transcripts in the biological sample. Such a qualitative and quantitative method to characterize at least one RNA sequence read in a transcriptome dataset for fusion transcripts is disclosed, comprising the steps of:

(a) providing a computer for data identification, aligning, comparison and computation purposes, wherein: the computer has access to the transcriptome dataset, the transcriptome dataset comprising data of genome-wide RNA sequence reads and counts thereof and; and the computer has access to a predetermined fusion transcript table, the predetermined fusion transcript table comprising data of predetermined E5-E3 keys, wherein: each of the predetermined E5-E3 keys corresponds to junction sequence of a predetermined fusion transcript, comprising an E5 key and an E3 key, wherein the E5 key corresponds to a 5′-end subsequence of the predetermined fusion transcript and is mapped to a first annotated gene; the E3 key corresponds to a 3′-end subsequence of the predetermined fusion transcript and is mapped to a second annotated gene; and the E5 key and the E3 key is connected at a junction of the predetermined fusion transcript;

(b) aligning the at least one RNA sequence read with each of the E5-E3 keys in the predetermined fusion transcript table; and

(c) determining that the at least one RNA sequence read is mapped to a predetermined fusion transcript if the at least one RNA sequence read contains a subsequence substantially identical to an E5-E3 key in the predetermined fusion transcript table.

Optionally in some embodiments, the method may further comprise, following step (c), a step of determining expression level of the predetermined fusion transcript to which the at least one RNA sequence read is mapped in the biological sample, the step comprising: (i) determining that E5 key and E3 key of the E5-E3 key, which corresponds to the predetermined fusion transcript, are unique in the transcriptome dataset; and (ii) determining the expression level of the predetermined fusion transcript in the biological sample, by dividing the count of the at least one RNA sequence read by sum of the counts of the genome-wide RNA sequence reads in the transcriptome dataset.

This disclosure also provides all the fusion transcripts identified by the above mentioned method applied in human cancer cells, with their junction sequences specifically disclosed herein.

A set of isolated, cloned recombinant or synthetic polynucleotides, is provided herein, comprising at least one polynucleotide, wherein each of the at least one polynucleotide encodes a fusion transcript, the fusion transcript comprising a 5′ portion from a first gene and a 3′ portion from a second gene, wherein the 5′ portion from the first gene and the 3′ portion from the second gene is connected at a junction; the junction has a flanking sequence, comprising a sequence selected from the group of nucleotide sequences as set forth in SEQ ID NOs: 1-258,853, or from complementary sequences thereof.

Disclosed herein also includes compositions and methods for detecting the presence of the fusion transcripts as disclosed above, based substantially on approaches to detect the above disclosed junction sequences of these fusion transcripts.

As such, this disclosure provides a composition for detecting, from a biological sample from a subject, the set of polynucleotides which correspond to the above disclosed junction sequences of the fusion genes.

In some embodiments, the composition may comprise at least one probe, wherein each of the at least one probe comprises a sequence that hybridizes specifically to a junction of a fusion transcript encoded by one of the set of polynucleotides. One such example may include one or more polynucleotide probes for Northern blot analysis to detect the presence of fusion transcripts. Another example may include a plurality of probes, which are immobilized on a substrate and used for microarray analysis to detect the presence of fusion transcripts.

Yet in some other embodiments, the composition may comprise at least one pair of probes, wherein each of the at least one pair of probes comprises: a first probe comprising a sequence that hybridizes specifically to a first gene of a fusion transcript encoded by one of the set of polynucleotides; and a second probe comprising a sequence that hybridizes specifically to a second gene of the fusion transcript. One example may include one or more pairs of hybridizing probes used in an in situ hybridization (ISH) assay to detect the presence of fusion transcripts.

Yet in some other embodiments, the composition may comprise at least one pair of amplification primers, wherein each of the at least one pair of amplification primers comprise a first amplification primer comprising a sequence that hybridizes specifically to a first gene of a fusion transcript encoded by one of the set of polynucleotides; a second amplification primer comprising a sequence that hybridizes specifically to a second gene of the fusion transcript; and a means for detecting an amplified product generated between the first amplification primer and the second amplification primer. One example may include a pair of amplification primers used for RT-PCR analysis to detect the presence of fusion transcripts. The composition as such may also comprise a means for generating cDNA molecules from mRNA molecules in the biological sample, such as a reverse transcriptase.

This disclosure further provides a method for detecting, from a biological sample from a subject, the presence of at least one of the set of polynucleotides which correspond to the above disclosed junction sequences of the fusion genes, comprising: (a) performing a biochemical assay on the biological sample, using at least one gene fusion informative composition for detection of the at least one of the set of polynucleotides; and (b) determining the presence, or absence, of the at least one of the set of polynucleotides in the biological sample.

In some embodiments of the method, the biochemical assay in step (a) comprises a nucleic acid hybridization technique, such as in situ hybridization (ISH), microarray analysis, and Northern blot analysis. In the embodiment where the biochemical assay in step (a) is a microarray analysis, the biochemical assay may comprise the sub-steps of: (i) isolating mRNA molecules from the biological sample; (ii) converting the mRNA molecules into cDNA molecules, and optionally amplifying the cDNA molecules; (iii) labeling the cDNA molecules; (iv) hybridizing the labeled cDNA molecules to a microarray chip, wherein the microarray chip comprises a plurality of probes and a substrate; the plurality of probes are immobilized on the substrate; and each of the plurality of probes comprises an oligonucleotide sequence that hybridizes specifically to a junction of a fusion transcript encoded by one of the set of polynucleotides; and (v) detecting a pattern of hybridization for each of the plurality of probes.

Yet in some other embodiments of the method, the biochemical assay in step (a) comprises a nucleic acid amplification technique, selected from the group consisting of: polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). In the embodiment where the biochemical assay is reverse transcription polymerase chain reaction (RT-PCR), the biochemical assay in step (a) comprises the sub-steps of: (i) isolating mRNA molecules from the biological sample; (ii) converting the mRNA molecules into cDNA molecules; (iii) performing at least one PCR on the cDNA molecules, using at least one pair of amplification primers, wherein each of the at least one pair of amplification primers comprise a first amplification primer comprising a sequence that hybridizes specifically to a first gene of a fusion transcript encoded by one of the set of polynucleotides; a second amplification primer comprising a sequence that hybridizes specifically to a second gene of said fusion transcript encoded by one of the set of polynucleotides; and (iv) detecting amplification products from the at least one PCR.

In some embodiments of the method, the biochemical assay in step (a) comprises a nucleic acid hybridization technique, such as in situ hybridization (ISH), microarray analysis, Northern blot analysis, and RNA CaptureSeq. In the embodiment where the biochemical assay is RNA CaptureSeq, the biochemical assay in step (a) comprises the sub-steps of: (i) isolating mRNA molecules from the biological sample; (ii) designing DNA oligonucleotide probes specific to splicing junctions of fusion transcripts; (iii) propagating cDNA libraries; (iv) hybridizing libraries to probes; (v) washing and removing no targeted cDNA; (vi) eluting targeted cDNA for sequencing; and (vi) analyzing captureseq data described above.

### BRIEF DESCRIPTION OF THE DRAWINGS

FIG. 1 shows schematic diagram of classification of different types of alternatively-spliced isoforms and fusion transcripts. 1 and 2, 3 are upstream, middle and downstream introns. The white, gray and black squares represent upstream, middle and downstream exons, respectively. Reference (REF) is a verified annotated sequence and is used to generate splicing code table. Horizontal arrows indicate alternative splice sites. Vertical arrows indicate junctions of pre-mRNA splicing. A) The sequence is identical to the reference sequence. B) The sequence has no middle exon to form a novel intron. C) The sequence has identical 3′ splice site, but 5′ splice is different from the reference. Splicing generates a 5′ alternatively-spliced isoform. D) The sequence has identical 5′ splice site, but 3′ splice is different from the reference. Pre-mRNA splicing forms a 3′ alternatively-spliced isoform. E) The sequence has both different 5′ and 3′ splice sites. This is a novel intron. F) Two different transcriptional units are originally transcribed separately into different molecules. Genetic alternations have brought two genes together to form a new transcriptional unit and to generate fusion transcripts. Alternatively, trans-splicing generates a fusion transcript.

FIG. 2 shows schematic procedure of using the splicingcode model to analyze RNA-seq data. The splicingcode program can generate three different tables, which are E5-E3 table, E5 table and E3 table. Using these three tables, we can obtain the most important information of RNA-seq data. The black arrows indicate directions. Horizontal arrows represent two pathways: identification of novel splicing isoforms and discovery of fusion transcripts.

FIG. 3 shows a detailed description of the method to identify fusion transcripts from RNA-seq reads, shown in the right pathway in FIG. 2.

FIG. 4 shows detailed characterization of the 16,570 fusion transcripts with canonical splice junctions identified from ENCODE from thirty-nine cancer cell line datasets (ECD39). FT and PFG represent fusion transcripts and putative fusion genes supported fusion transcripts, reprehensively. a) Characterization of the fusion transcripts identified from ENCODE thirty-nine cancer cell lines (ECD39). White bar represents total 16,570 fusion transcripts. Some of fusion transcripts are alternatively spliced from the two same putative fusion genes indicated by gray bar. Black bar and gray doted bar represent numbers of 5′ unique genes and 3′ unique genes, respectively. The numbers reduced from total PFG's numbers indicate 5′ and 3′ gene redundancies, which suggest the numbers of genes can be fused two or more different genes. Dark doted gray bar shows the total numbers of unique genes of both 5′ and 3′ genes, reduction of which indicates a gene can be used as a donor or as an acceptor. Black and gray bars in the Insert of FIG. 1a represent average numbers of sequence reads across splice junctions and average lengths of fusion transcripts, respectively. b) Distribution of fusion transcripts in 39 cancer cell lines. Gray, black, and white bars represent the putative fusion genes, fusion transcripts and the millions of sequence reads used to identify fusion transcripts; c). Type distributions of fusion transcripts. Gray and black bars indicate the putative fusion genes and fusion transcripts, respectively; d). Distributions of cancer cell lines in which fusion transcripts have been identified. Gray, dark gray and black bars represent percentages of fusion transcripts that are detected in 1, 2 and ≧3 cancer cell lines, respectively.

FIG. 5 shows a Van diagram of overlapped fusion transcripts between different datasets. In this paper, “overlapped” means “identical”. Gray and white circles represent the ECD39's MCF7 fusion transcripts we have identified and those fusion transcripts validated by Sakarya et al. (Sakarya, et al. 2012).

FIG. 6 shows Van diagrams of overlapped fusion genes between ECD39 and GCD. a). Van diagram showing identical (overlapped) fusion genes between the ECD39 MCF7 fusion transcripts (dark gray) and the GCD MCF7 fusion transcripts (light gray); b). Van diagram showing identical (overlapped) fusion genes between the total ECD39 fusion transcripts (white circle) and the total GCD fusion transcripts (light gray).

FIG. 7 shows analysis and characterization of HMGA2|LUM fusion transcripts in osteosarcoma SJSA1 cell line, a multipotential sarcoma. a). Structures of HMGA2 and LUM genes, which are represented by black and gray arrows, respectively. Both genes are on chromosome 12 and separated by 25 Mb. They are brought together by deletions or translocations, which are indicated a pair of paralleled lines. Dashed white box indicates unknown regions between two gens. Black and gray squares represent exons of two different genes while triangle lines represent introns, respectively. Dashed line are omitted exons and introns. Dashed arrow indicates that two genes are close enough to be transcribed into a single molecule pre-mRNA; b) There are two fusion transcripts that differ by two nucleotides (isoform 1 vs isoform 2). c) Expression levels of these two isoforms (isoform 1 vs isoform 2) differ by 4200 folds.

FIG. 8 shows illustrations and experimental verification of the lowly-expressed CPSF6|CACNA1E fusion transcripts in lymphoblastoid cells GM12878. a). CPSF6 gene on the chromosome 12 and CACNA1E gene on the chromosome 1 have been brought together via translocation indicated by arrows. Black and gray squares represent exons to demonstrate where breakpoints are located on the genes. The numbers indicate exon positions. Solid angle lines and dashed dots represent introns and gaps, respectively. b). RNA-splicing has removed intronic sequences of the putative CPSF6|CACNA1E fusion gene. Black and gray capital letters represent 5′ and 3′ exonic sequences, respectively. Gray and black italic letters represent 5′ and 3′ intronic sequences, respectively. The numbers indicate sequence gaps. c). Diagrams show that the CPSF6|CACNA1E fusion transcript is amplified by RT-PCR. cDNA fragments are then cloned into pCR4-TOPO clone vector. The positive clones are sequenced. The fusion transcripts are verified by blast and visual inspections. Arrow indicates splice junction of the CPSF6|CACNA1E fusion transcripts. Black and gray squares represent CPSF6 exons and CACNA1E exons, respectively.

FIG. 9 shows analysis and characterization of MTG1|SCART1 (LOC609217) read-through fusion transcripts. a). Schematic diagram of structures of MTG1 and SCART1 genes on the chromosome 10q26.3. The black and dark gray arrows represent MTG1 and SCART1 genes, respectively. Other genes around MTG1 and SCART1 genes are indicated by white and light gray arrows. Dashed lines represent omitted exons and introns. Dashed arrow indicates read-through transcription of a single pre-mRNA molecule, which is spliced into fusion transcript; b) There are eight MTG1|SCART1 fusion transcripts identified, which are shown to be alternatively spliced; The black and gray boxes represent MTG1 and SCART1 exon, respectively. The numbers above the boxes are exon numbers. The numbers in the sequence indicate numbers of omitted nucleotides; c) Distribution of eight MTG1|SCART1 fusion transcripts. Black bars represent the numbers of eight MTG1|SCART1 fusion transcripts detected, respectively. d). Distribution of the total MTG1|SCART1 fusion transcripts detected among different cancer cell lines; and e). Distribution of the normalized MTG1-SCART1 fusion transcripts among different cancer cell lines. Y-axe unit is numbers of transcripts per million sequence reads (NSJMR).

FIG. 10 shows differential expression of read-through C19orf47|AKT2 fusion transcripts. a). The C19orf47|AKT2 fusion transcripts have been detected in nine normal tissues, which include bone marrow (b. marrow), colon, duodenum, fallopian tubes (f. tube), fat gall bladder (g. bladder), testis, thyroid, tonsil and not found in 20 other tissues including breast and HMEC; b). The C19orf47|AKT2 fusion transcripts have been observed in 9 samples out of 168 HIBCD breast cancer samples. The expressional levels of the C19orf47|AKT2 fusion transcripts are expressed in NSJMR (numbers of splice junctions per million reads).

FIG. 11 shows analysis of read-through GAL3ST2|NEU4 fusion transcripts. The GAL3ST2|NEU4 fusion transcripts have been found to be expressed only in normal colon tissues, but absent in 26 other tissues and HMEC. This demonstrates that GAL3ST2|NEU4 are differentially expressed. The GAL3ST2|NEU4 fusion transcripts have been detected in 5 different individual cancer tissues. The expressional levels of the GAL3ST2|NEU4 fusion transcripts are expressed in NSJMR (numbers of splice junctions per million reads).

FIG. 12 shows analysis and characterization of KANSL1 (KIAA1267)|ARL17A fusion transcripts. a). Schematic diagram of structures of ARL17A and KANSL1 genes on the chromosome 17. A potential inversion results in KANSL1-ARL17A gene structure. The gray and black arrows represent the KANSL1 and ARL17A genes, respectively. Dashes arrow indicate potential fusion pre-mRNA; b) there are six KANSL1|ARL17A fusion transcripts identified from cancer cell lines. Black and gray capital letters represent 5′ and 3′ exonic sequences, respectively. The numbers within the sequences indicate the omitted nucleotides; c) Distribution of six KANSL1|ARL17A fusion transcripts detected; d). Distribution of the total KANSL1|ARL17A fusion transcripts among different cancer cell lines; and e). Expression of the normalized KANSL1|ARL17A fusion transcripts among different cancer cell lines. Y-axe unit is numbers of splice junctions per million of sequence-reads (NSJMR). The black and gray boxes represent KANSL1 and ARL17A exons, respectively. Dashed lines indicate omitted exons and introns. The numbers above the boxes are exon numbers.

FIG. 13 shows an example of using the fusion transcripts' hit maps of fusion transcripts to identify genetic rearrangement hotspots. a). Distribution of total fusion transcripts and inversion fusion transcripts along the chromosome 17. b). Distribution of total fusion transcripts and inversion fusion transcripts found in ≧2 cancer cell lines along the chromosome 17. Each X-axe unit represents 5M bp. Arrows indicate the locations of KANSL1|ARL17A fusion transcripts. The gray triangles and black squares represent total fusion transcripts and inversion fusion transcripts, respectively.

FIG. 14 shows genome-wide hit maps of fusion transcripts. Relationship between total putative fusion genes (gray triangles) and putative inversion fusion genes whose transcripts existed in two or more cancer cell lines (black squares). a, b, c, d, e, f, g, h, i, j, k, l, m, n, o, p, q, r, s, t, u, v and x represent human chromosome 1, 2, 3, 4, 5, 6, 7, 8, 9, 10, 11, 12, 13, 14, 15, 16, 17, 18, 19, 20, 21, 22, and X. Each of X-axe units represents 5 Mb.

FIG. 15 shows results of comparative analyses of numbers of KANSL1|ARL17A samples between HIBCD and SKBCP datasets. Gray and black squares represent total numbers of samples and the numbers of samples that are found to have KANSL1|ARL17A, respectively. The difference of KANSL1|ARL17A samples between HIBCD and SKBCP is found to be statically significant (p<0.001).

FIG. 16 shows expressions of KANSL1|ARL17A fusion transcripts in the 168 HIBCD breast cancer samples. X-axe indicates samples' IDs. Y-axe is numbers of splice junctions per million reads (NSJMR).

FIG. 17 shows results of analysis of the 168 HIBCD (a) and SKBCP (b) breast cancer samples and identification of GABBR1andUBD|PSPH fusion transcripts. a). The GABBR1andUBD|PSPH fusion transcripts have been found in 31 HIBCD samples. b). The GABBR1andUBD|PSPH fusion transcripts have been detected in 7 SKBCP samples. Y-axe is NSJMR.

FIG. 18 shows verification results of the low-level expressed GABBR1andUBD|PSPH fusion transcripts in breast cancer cell line BT-474. a) GABBR1andUBD gene is located on chromosome 6 and has 24 exons while PSPH genes is on chromosome 7 and has 8 exons. Black and gray squares represent GABBR1andUBD exons demonstrate where breakpoints are located on the genes. Dark and light gray boxes represent PSPH exons to demonstrate where breakpoints are located on the genes. A potential translocation results in putative GABBR1andUBD|PSPH fusion gene, which is represented by black and light gray boxes; b). Black Capital and dark italic gray letters represent exonic and intronic sequences of GABBR1andUBD 5′ splice junction sequences. The light gray italic and gray Capital letters are intronic and exonic sequences of the PSPH 3′ fusion junction; c). These GABBR1andUBD|PSPH fusion transcripts are amplified by RT-PCR. d). RT-PCR fragments are then cloned into pCR4-TOPO clone vector. The positive clones are isolated and sequenced. The arrow indicates splice junctions of the GABBR1andUBD|PSPH fusion transcripts. The black and light gray boxes represent GABBR1andUBD and PSPH exons, respectively.

FIG. 19 illustrates complex fusion transcripts between non-coding RNA oncogene PVT1 and protein-coding EXOC4 genes. a). A rod-like structure shows that EXOC4 gene is located on Chromosome 7. Gray boxes and black line triangles represent exons and introns, respectively; b). A rod-like structure shows that non-coding RNA PVT1 gene is located on chromosome 8q24 and has been shown to be an non-coding RNA oncogene. The black boxes and triangle lines indicate PVT1 gene structure; c) PVT1|EXOC4 fusion transcripts. 9 fusion transcripts have been identified have been identified in SH-N-SK cancer cell line, a human neuroblastoma. The black and gray rectangle boxes represent the PVT1 and EXOC4 exons, respectively. d) Differential Expression of PVT1|EXOC4 fusion transcripts; e). EXOC4|PVT1 fusion transcripts have been detected in SH—N-SK cancer cell lines. The black and gray rectangle boxes represent the PVT1 and EXOC4 exons, respectively; f) Differential Expression of EXOC4|PVT1 fusion transcripts; g). Expression comparison between EXOC4|PVT1 and EXOC4|PVT1 fusion genes. The gray and black bars represent the PVT1|EXOC4 fusion gene and EXOC4|PVT1 fusion gene, respectively. Y-axe unit is numbers of fusion transcripts. Since these fusion transcripts come from the same dataset, they reflect the differences of these fusion transcript expressions.

FIG. 20 shows analysis and characterization of non-coding RNA-RNA fusion transcripts. a). The gray and black arrows MEG8 and SNORD114-1 genes respectively. The dashed arrow shows potential inversions or regional duplications of chromosomal 14q32.31 have resulted in inversion of MEG8 and SNORD114-1 gene orders to generate putative SNORD114-1|MEG8 fusion genes; b) Five SNORD114-1|MEG8 fusion transcripts have been detected; c) Distribution of total SNORD114-1|MEG8 fusion transcripts. SNORD114-1|MEG8 fusion transcripts have been detected in seven cancer lines; and d) Distribution of normalized SNORD114-1|MEG8 fusion transcripts in seven cancer lines. Y-axe unit is numbers of transcripts per million sequence reads (NSJMR). The black and gray rectangle boxes represent SNORD114-1 and MEG8 exons, respectively. Here, SNORD114-1 and MEG8 represent abbreviated SNORD114-1andSNORD114-2andSNORD114-3 gene and MEG8andSNORD112andSNORD113-3 gene, respectively.

FIG. 21 shows results of analysis and characterization of non-coding RNA fusion transcripts. a). Distribution of non-coding RNA fusion transcripts (gray) and PFG (black) among different classes of non-coding RNA fusion transcripts. b) Distribution of -coding RNA fusion transcripts (gray bars) and PFG (black bars) among different cancer cell lines; c) Distribution of different SNHG fusion transcripts. d). Distribution of SNHG3 fusion transcripts among different cancer cell lines; e). Comparison of upstream (gray bars) and downstream (black bars) SNHG fusion transcripts; and f). Comparison of upstream (gray bars) and downstream (black bars) natural networks formed by fusion transcripts.

FIG. 22 shows diagrams of verification of the lowly-expressed ncRNA00188|GNAI3 fusion transcripts in lymphoblastoid cells GM12878. a). ncRNA00188 gene is located on the chromosome 17 and codes for a non-coding RNA. GNAI3 gene is on the chromosome 1 and a protein-coding gene. Two genes have been brought together via translocation indicated by arrows. Black and gray boxes represent ncRNA00188 exons to demonstrate where breakpoints are located on the ncRNA00188 gene. Black and white boxes represent GNAI3 exons to demonstrate where breakpoints are located on the GNAI3 gene. The numbers indicate above the boxes exon positions. Solid angle lines and dashed dots represent introns and gaps, respectively. b). RNA-splicing has removed intronic sequences of the putative ncRNA00188|GNAI3 fusion gene. Black italic letters and Capital gray letters represent 3′ intronic and 3′ exonic sequences of the GNAI3 splice junction, respectively. The numbers within the sequence indicate sequence gaps. c). Diagrams show that the ncRNA00188|GNAI3 fusion transcript is amplified by RT-PCR. cDNA fragments are then cloned into pCR4-TOPO clone vector. The positive clones are sequenced. The fusion transcripts are verified by blast and visual inspections. Arrow indicates splice junction of the ncRNA00188|GNAI3 fusion transcripts. RT is RT-PCR amplification of GM12878 cDNAs. No products have been detected in other cancer cell lines. M represents DNA markers.

### BRIEF DESCRIPTION OF THE SEQUENCE LISTING

The instant disclosure includes a plurality of nucleotide sequences. Throughout the disclosure and the accompanying sequence listing, the WIPO Standard ST.25 (1998; hereinafter the “ST.25 Standard”) is employed to identify nucleotides. The sequences of SEQ ID NOs: 1-258,077 are novel fusion transcripts. The sequences of SEQ ID NOs: 258,078-258,853 may have overlapped with Gene IDs of Mitelman Database of Chromosome Aberrations and Gene Fusions in Cancer (Mitelman, et al. 2015). The sequences from SEQ ID NOs: 258,854-259,170 have identical splice junctions to those of the fusion transcripts that have been published.

### DETAILED DESCRIPTION

Previously, we have observed that recently-gained human spliceosomal introns have identical 5′ and 3′ splice sites (Zhuo, et al. 2007). Based on this finding, we have found that both 5′ exonic sequences (E5) immediately upstream of introns and 3′ intronic sequences (13) are dynamically conserved and appears rather reminiscent of self-splicing group II ribozymes and of constraints imposed by base pairing between intronic-binding sites (IBSs) and exonic-binding sites (EBSs) (Zhuo, et al. 2012). Therefore, we have proposed that both E5 and 13 sequences constitute splicing codes, which are deciphered by splicer proteins/RNAs via specific base-pairing (Zhuo, et al. 2012). Our splicing code model suggested that a yet-to-be characterized splicer proteins/RNA would decode identical sequences in all pre-mRNAs in conjugation with U snRNAs and spliceosomes, regardless whether the E5 and 13 sequences are in the one molecule or two different molecules.

In order to generate splicingcode tables, we first and 2010 exons/introns coordinates file are downloaded from the NCBI AceView (ACEVIEW 2010) and the human hg19 genome sequences from UCSC (UCSC 2014). The sequences from the splicing sites have been parsed out by a software program. Generating the human splicing codes have been described in details in U.S. patent application Ser. No. 13/372,180 filed on Feb. 13, 2012 and titled SYSTEM AND METHOD FOR ANALYZING SPLICING CODES OF SPLICEOSOMAL INTRONS. Briefly, we divided 5′ splice site and 3′ splice sites. Starting from the splicing junctions, 5′ splice site are further divided into its 5′ exonic sequence (E5) and 5′ intronic sequence (I5). Similarly, 3′ splice site is divided into 3′ intronic sequence (13) and 3′ exonic sequence (E3). Starting from the splice junction, we scored the length of identical nucleotides (LIN) in an uninterrupted stretch independently for the E5-I3 and I5-E3 alignments. The total LIN of splice sites is sum of the LINs of the E5-I3 and I5-E3 alignments. To increase the quality of fusion transcripts, we removed the introns with LIN≧10 from the splicing codes. Furthermore, we arbitrarily removed all introns with lower-case letters to further improve the quality of fusion transcripts in this study. These two steps reduce to unique introns to 308,854, which are used to measure gene expression. To further reduce redundant E5 and E3 sequences, we only retained introns whose E5 splice sites or E3 splice sites can have maximum of 20 isoforms. Consequently, we reduced the unique E5 sequences to 229,170 and unique E3 sequences to 213,327.

For the program convenience and clarity, we use the human splicing codes to generate E5-E3 hash tables. Then we use E5-E3 table to generate an E5 table and an E3 table. These three tables have different types of keys, but are associated with a unique ordered value. Selecting the key lengths of the E5-E3 table depends on length of RNA-seq reads. If key lengths are too short, it will put multiple sequences from different genes into on one exon-exon junction. It will increase error if these exon-exon junctions are used to evaluate gene expression patterns. If it is too long, it will increase the quality of the expression data. It may result in less of data points and loss of information especially if lengths of RNA-seq reads are variable. Generally, we have used 20 bp unless they are specified in the context. We have used this E5-E3 table to generate an E5 table and an E3 table.

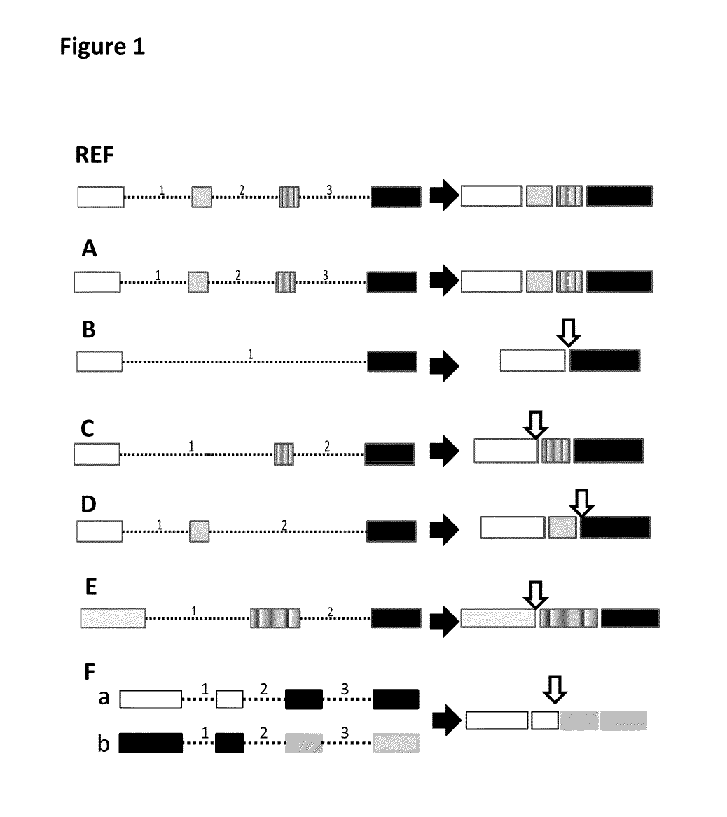

In order to be more efficient and accurate to get the most important information of the entire transcriptome, we must correctly identify their splicing junctions. RNA-Seq reads without splicing junctions are less important and contribute a little to their reconstructing genes. Therefore, we will evaluate these RNA-Seq reads further if necessary. In order to get more accurate identification of different classes of splice junctions in RNA-seq datasets, we have selected a well-annotated mRNAs from each gene as reference sequences (REF) shown in FIG. 1. RNA-seq sequences are then searched to see whether they have identical E5-E3 junctions or E5 sequences or E3 portions. If they have either E5 or E3 portions, they may be potential novel isoforms or fusion transcripts. Splicing of uncharacterized introns marked by vertical arrows in FIG. 1 can be classified into following five types of splicing junctions: A) identical introns; B) cassette introns; C) 5′ alternative introns; D) 3′ alternative introns; and E) novel introns. In FIG. 1F, two transcriptional units or genes may be located on different chromosomes or on different regions of the same chromosome. Inter-chromosomal or intra-chromosomal translocations have brought two transcriptional units close each other to generate a fusion gene, which in turn are transcribed into fusion transcripts. In some other cases, two transcriptional units may be separated by relatively short stretches of sequences (30 Kb-1,000 kb). However, under certain conditions and/or in some tissues, the two transcriptional units are transcribed into one unit to generate fusion transcripts. In other cases, two RNAs from two different molecules are trans-spliced to generate fusion transcripts.

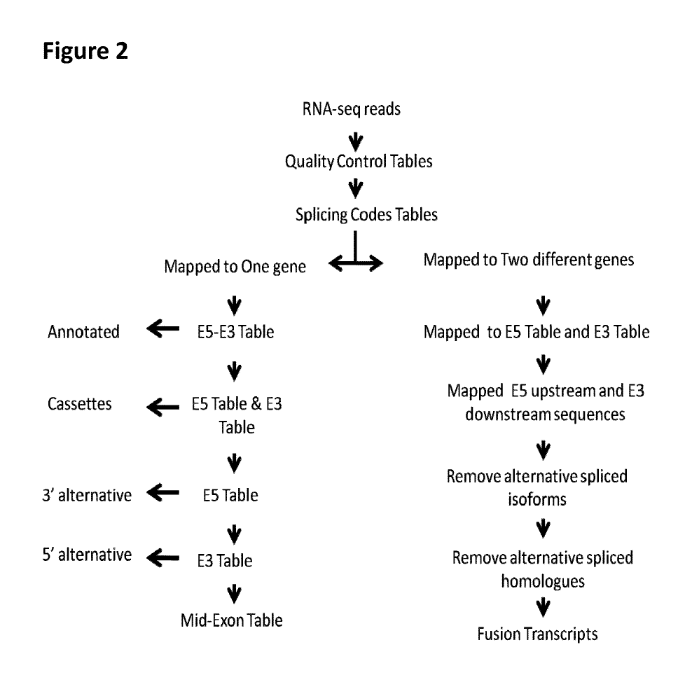

Since our goal is to generate high-quality data, novel isoforms and fusion transcripts, we have to reduce the most noises first. As shown in FIG. 2, in the first step, we have used Quality Control Table to remove mitochondrial gene sequences, mitochondrial ribosomal RNAs, ribosomal RNA sequences, simple sequences, such as poly (A) sequences, GC-repetitive sequences and AT-rich sequences found in the human genomes, and another other sequences, which are thought to be contaminants. To generate Quality Control Table, the selected sequences are used to generate continuous ordered keys. Each key is associated with upstream and downstream sequences, which are used to confirm whether the key is in correct context of the associated sequences. Even though all samples have been rRNA-depleted, we have found that the samples contains up to 20% of ribosomal rRNA sequences and mitochondrial gene sequences. More importantly, we can use this table to remove poor-quality RNA-Seq reads, simple repeat sequences and adaptor sequences.

If a sequence is found to have a substring present in this E5-E3 hash table, the read's remaining sequence will be aligned to the corresponding E5 and E3 exonic sequences perfectly or with errors or gaps set by users such as one nucleotide. If the sequence reads match both E5 and E3 sequences from the same splice junctions, these reads will be accounted for gene expression profiling. Otherwise, they are treated as poor-quality reads or as novel transcripts for further analysis. Then we have used both E5 table and E3 table to identify novel alternatively-expressed transcripts and fusion transcripts.

If RNA-seq reads are mapped into both E5 table and E3 table, but not from the same splice junctions, then they have two different pathways as shown in FIG. 2. If both E5 key and E3 key are from the same gene or transcriptional unit (the identical gene ID), they are novel alternative splicing. If both E5 key and E3 key are associated with different gene IDs or transcriptional units, they are potentially fusion transcripts and will be described in detail later.

If both E5 and E3 keys have the same gene ID and from the same transcription units, then we can check the orders of both E5 key and E3 key to determine types of alternative splicing.

If a RNA-seq read has been mapped on the same transcriptional unit, there are two or more gaps between the E5 ID value and the E3 ID value. Two more exons have been removed from transcripts. This RNA-seq read is cassette introns as shown by vertical arrow (Type B in FIG. 1).

The sequence has a subsequence in the E5 table and its immediate downstream sequences are mapped to an E3 key associated with a different value. The transcript sequence is thought to have identical 5′ splice site, but has different 3′ splice site. This sequence is thought to have 3′ alternative splicing as the intron 1 shown in Type C in FIG. 1.

If the transcript is present in the E5 table and length of its downstream sequence is more than the key length, these sequences will be searched by blast to determine the sequence location. If the sequences are located within the downstream gene or downstream sequences of the transcription unit, this sequence is thought to be 3′ alternative splicing. If the sequences are located in another transcription unit, this sequence is thought to be a fusion transcript.

If the transcript is present in the E3 table and its immediately upstream sequence is more than the key length, these sequences will be searched by blast to determine the sequence location. If the sequences are located within the upstream gene or upstream sequences of the transcription unit, this sequence is thought to be 5′ alternative splicing. If the sequences are located in another transcription unit, this sequence is thought to be a fusion transcript.

If a RNA-seq read has been mapped to the E3 key, its immediately upstream sequence is mapped to the E5 key with different value. That is, a sequence has identical 3′ splice site with the REF sequence, but has different 5′ splice site, this sequence is thought to have 3′ alternative splicing as shown in Type D in FIG. 1.

If the transcript is present in the E3 table and the length of its upstream sequence is more than the key length, these sequences will be searched by blast to determine the sequence location. If the sequences are located within the upstream gene or upstream sequences of the transcription unit, this sequence is thought to be 5′ alternative splicing. If the sequences are located in another transcription unit, this sequence is thought to be a fusion transcript.

If the E5 key and E3 key are mapped to keys with different values compared to their REF sequences, this transcript has different 5′ and 3′ splice sites compared to the reference sequence (REF in FIG. 1). The intron 1 of the Type E has been shown to be a novel intron in FIG. 1. If the transcript is present in the E3 table and the length of its upstream sequence is more than the key length, these sequences will be searched by blast to determine the sequence location. If the sequences are located within the upstream gene or upstream sequences of the transcription unit, this sequence is thought to be 5′ alternative splicing. If the sequences are located in another transcription unit, this sequence is thought to be a fusion transcript.

In order to assemble the transcriptome and to characterize novel and unpredictable transcriptional events, we have added middle exon table in this RNA-seq analysis program. To generate the middle exon table, we have adopted one of two strategies deepening on the computer system memories and lengths of RNA-seq reads: continuous non-redundant and unique keys or gapped (normally less than half of the key length) non-redundant and unique keys. RNA-seq reads are mapped into the middle exon table.

To measure the gene expression, we have adopted splice junction centered strategy. That is, we would count the sequence reads covering splice junctions and ignore all other parts of mRNA sequences. We first selected the human 308,854 splice junctions from human Aceview 37 genes from 382,279 distinct exon/intron junction sequences as described above. As described above, we removed the introns with LIN ≧10 from the splicing codes. We arbitrarily removed all introns with lower-case letters to further improve the quality of fusion transcripts in this study. These two steps reduce to unique introns to 308,854, which are used to measure gene expression. We have combined 20 bp E5 and 20 bp E3 key sequences as unique splice junction database. RNA-seq reads are searched against this human splice junction database. If a sequence read contain sequences in the splice junction database, this splice junction is counted. To be consistent with identification of fusion transcripts, we allow no mismatches. To quantify gene expression levels, we summed the total numbers of sequence reads per gene. The numbers of the splice junctions we have identified are divided by the sums of sequence reads. The results are expressed in Numbers of Splice Junction per Million mapped Reads (NSJMR).

In order to measure expression of the fusion transcripts identified so far, we have adopted a strategy similar to measure gene expression described above. We have divided the fusion transcripts into E5 and E3 sequences from fusion junctions as described above. We have taken a substring of an E5 sequence as the E5 key and a substring of an E3 sequence as E3 key. Both E5 and E3 keys of the same fusion transcripts are combined together to form a join key of a fusion transcript. The length of each of both E5 and E3 keys are at least 20 bp to make sure that the joint key will be unique in a transcriptome. If a sequence contains this joint key, this sequence is counted as a fusion transcript, the numbers of this fusion transcript are summed together in a dataset. The numbers of the fusion junctions we have identified are divided by the sums of sequence reads of the dataset. The results are expressed in Numbers of Splice Junction per Million mapped Reads (NSJMR).

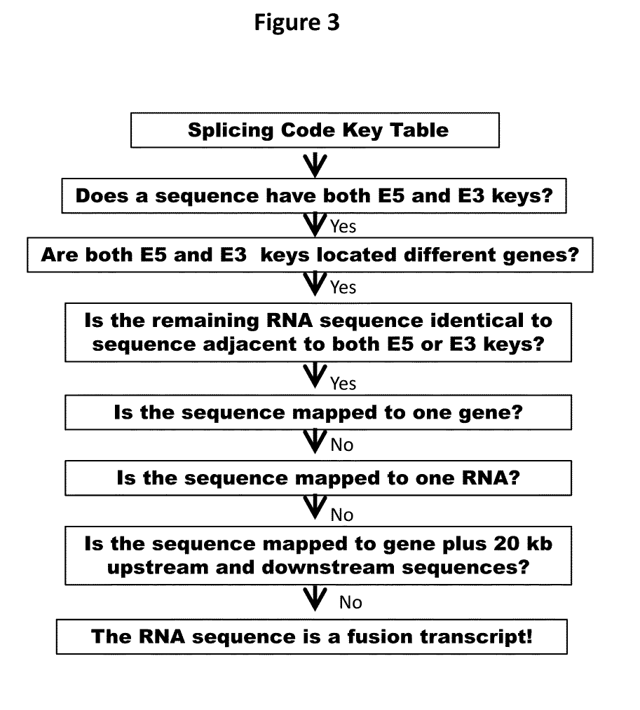

As shown in FIG. 2, when a sequence read is mapped to E5 table and its immediately downstream key is mapped to an E3 key of different gene, this sequence read is thought to be a putative fusion transcript. Due to enormous importance of fusion transcripts, we have given more detailed description to discover fusion transcripts in FIG. 3. After we have found that a sequence have both E5 and E3 keys on different genes, we will further check whether 5′ RNA-seq read sequences have identical sequences upstream of the E5 key sequence and if 3′ remaining read sequence match an identical sequence downstream of the E3 key sequences. If a read sequence has identical E5 and E3 sequences from two different genes, this read sequence are further checked by BLAST against the mRNA database to see if they come from pseudogenes or from gene duplications or from alternative splicing. If the RNA-seq read doesn't originate from one single transcription unit, this fusion sequence is searched against E5 and E3 gene sequences via gene hash tables to rule out whether the fusion transcript comes from alternative splicing. The entire identification process of fusion transcripts has used zero tolerance of errors in this study. The fusion transcripts have been randomly selected and verified by manual inspections. In addition, the fusion transcripts are systematically BLASTed against AceView mRNA sequences and BLASTed against human genes parsed from human hg19 genome sequences to make sure that each of the fusion transcripts originates from two different genes.

To use the splicing code to identify fusion transcripts, a computation system used three steps: 1) mapping a sequence read to 20 bp 5′ (E5) and 20 bp 3′ (E3) exonic sequences of canonical splice-sites of two different transcription units; 2) aligning remaining sequences to corresponding upstream and downstream regions; and 3) removing alternatively-spliced false positive sequences from one transcription unit by blast against mRNA and gene databases. These steps have shown that splicing code table is the key to determine qualities of fusion transcripts. We have downloaded AceView-NCBI-37 genes, which contain 382, 279 distinct introns (Thierry-Mieg and Thierry-Mieg 2006). After removing introns from intergenic regions and E5 or E3 sequences whose frequencies are larger than 20, the table contained 221,970 E5 sequences and 213,327 E3 sequences, respectively. A sequence is mapped to E5 and E3 keys from two different genes. Then, the upstream sequence of the E5 key and the downstream of the E3 key are aligned to the corresponding genomic regions, respectively. If they are identical, this sequence is thought to be a fusion transcript. Consequently, our system would greatly reduce randomly generated false positive sequences, but also remove some true fusion transcripts. The maximum random error to generate a fusion transcript is 1.2×10⁻²⁴ and the medium error is 1×10⁻⁵⁹.

Using this computation system, first we have analyzed 37,208 millions of RNA-seq reads from thirty-nine cancer lines, majorities of which are downloaded from ENCODE project (ENCODE 2015). RNAs data sizes range from 31 millions of MDA-MB-231 to 6945 millions of MCF-7. For convenience, we have assigned these 16,570 fusion transcripts as Encode Cancer 39 Datasets (ECD39 Dataset) (ENCODE 2015). After we have analyzed ECD39 fusion transcripts and obtained summary information of the total fusion transcripts.

We have further downloaded four colon cancer datasets, two breast cancer datasets, two lung cancer data and normal tissues and primary cell lines (ENCODE 2015, SCILIFELAB 2015).

After we completed analyses of ECD39, we have continued analyzing the other cancer datasets downloaded from NCBI (ENA 2014) and ENA (ENA 2014). So far, we have identified total of 259, 170 fusion transcripts with unique canonical splice sites and represent 242,578 putative fusion genes. Then, we have downloaded the information from four large fusion transcripts, which include TCGA Fusion genes (Yoshihara, et al. 2014), Genentech's cancer fusion genes (Klijn, et al. 2015), Life Technology′ breast cancer fusion transcripts (Sakarya, et al. 2012) and Mayo Clinic Rochester's breast cancer fusion genes (Asmann, et al. 2012). We have parsed out >14,000 fusion transcripts from these fusion gene data. We have shown that 317 transcripts out of 253,747 fusion transcripts have identical fusion junctions. Next, we have compared our unique IDs with Mitelman Cancer Fusion Gene Database (Mitelman, et al. 2015), which contains 10,004 fusion genes so far. We have identified 776 fusion transcripts, whose Gene IDs are overlapped with those from Mitelman Cancer Fusion Gene Databases (Mitelman, et al. 2015). These have demonstrated that most of the fusion transcripts are novel and unique. Since the majorities of 39 cancer cell lines are from ENCODE projects (Table 1), their data handling and experimental error controls are uniforms. Because of these properties and characteristics of ENCODE datasets, it has made us much easier to remove mistakes and errors. The conclusions have been much reliable and reproducible. Therefore, our discussion will focus on this subset of datasets.

After we have performed analyses of the ENCODE RNA-seq datasets, we have discovered 92,817 fusion transcripts from these thirty-nine RNA-seq data, which represents 36.6% of the total fusion transcripts. In order to be more efficient to characterize the fusion transcripts, we have used them to analyze and dissect characteristics of fusion transcripts in more details and the other fusion transcripts are presented in the context of discussions, we have indentified 16,570 subset of fusion transcripts, which are supported by at least three sequences across the splice junction by minimum 40 bp (at least 20 bp at each of fusion transcripts) or by at least two alternatively-spliced fusion transcripts of the same two genes. For convenience, we have assigned these 16,570 fusion transcripts as Encode Cancer 39 Fusion Transcript Data (ECD39).

Table 1 has shown list of the thirty-nine cancer cell lines in the ECD39 datasets, the numbers of fusion transcripts (FT), the numbers of putative fusion genes (PFG), and the numbers of RNA-seq reads used for analyses.

TABLE 1
The information of the thirty-nine cancer cell lines (ECD39)
and their fusion transcripts identified. FT and PFG represent
fusion transcripts and putative fusion genes, respectively.

| Cancer Cell Lines | # of FT | # of PFG | # of Million Reads |
|---|---|---|---|
| A172 | 190 | 186 | 393 |
| A375 | 375 | 362 | 445 |
| A431 | 263 | 244 | 409 |
| A549 | 2053 | 1765 | 1933 |
| Caki2 | 146 | 142 | 447 |
| CUTLL | 554 | 455 | 462 |
| Daoy | 226 | 219 | 393 |
| G401 | 91 | 90 | 398 |
| H4 | 216 | 213 | 390 |
| H460 | 378 | 357 | 849 |
| HCC1599 | 442 | 387 | 230 |
| HCT116 | 422 | 403 | 498 |
| Hela-3 | 1177 | 1025 | 1977 |
| HepG2 | 2377 | 1886 | 5116 |
| HT1080 | 446 | 441 | 391 |
| HT29 | 392 | 382 | 465 |
| K562 | 3374 | 2572 | 3683 |
| Karpas422 | 211 | 205 | 293 |
| KATOIII | 128 | 111 | 186 |
| LHCN-M2 | 860 | 768 | 1391 |
| LIM1899 | 327 | 283 | 216 |
| LIM2405 | 87 | 76 | 248 |
| M059J | 206 | 203 | 327 |
| MCF7 | 2315 | 1763 | 6945 |
| MDA-MB | 114 | 105 | 31 |
| MG63 | 149 | 147 | 304 |
| OCI-Ly7 | 342 | 332 | 309 |
| PC3 | 317 | 311 | 437 |
| REC1 | 465 | 403 | 258 |
| RPMI-7951 | 420 | 406 | 345 |
| SJCRH30 | 565 | 530 | 380 |
| SJSA1 | 251 | 242 | 388 |
| SK-Mel-5 | 300 | 294 | 413 |
| SK-N-DZ | 826 | 799 | 1131 |
| SK-N-SH | 1731 | 1445 | 4622 |
| SUN16 | 55 | 47 | 138 |
| U251 | 33 | 33 | 110 |
| U2OS | 21 | 20 | 102 |
| U87 | 148 | 130 | 158 |

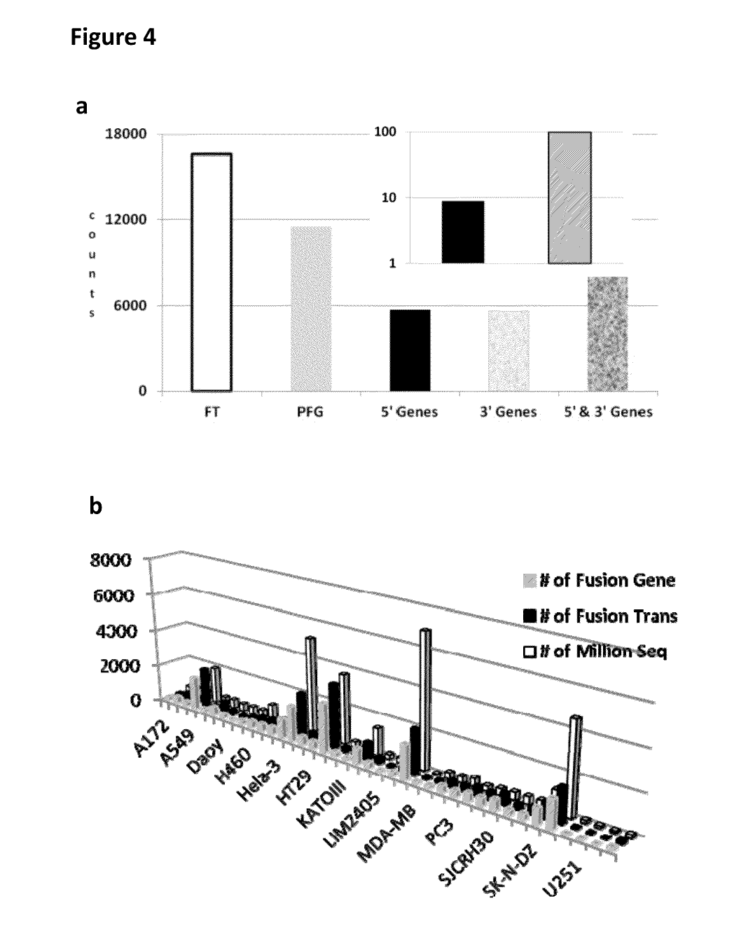

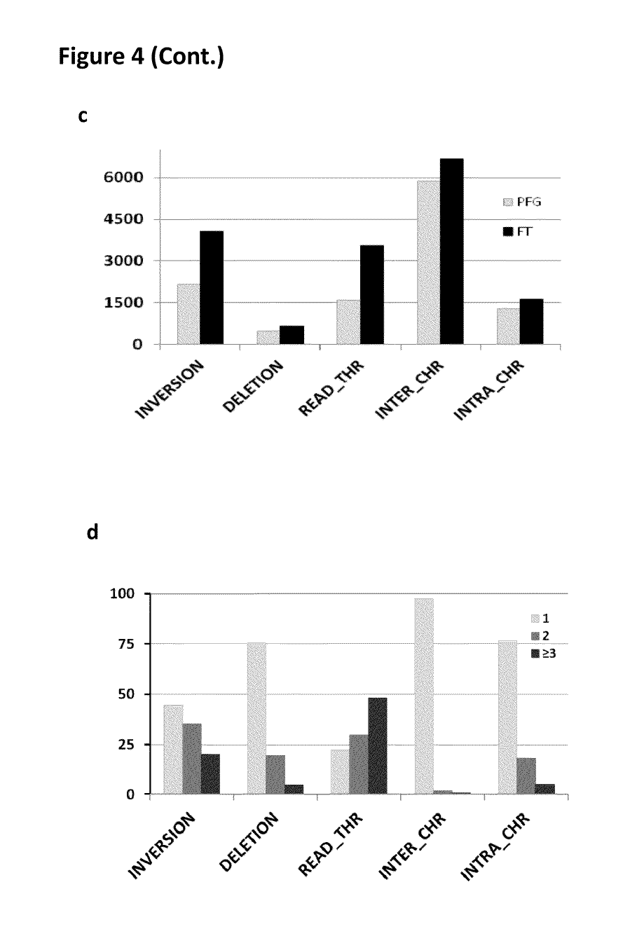

The ECD39 fusion transcripts have 16,570 fusion transcripts with canonical splice junctions which, on average, are supported by 8.9 copies of sequence reads and are 98 bp long (FIG. 4a Insert). These fusion transcripts represent 11,488 unique combinations of putative fusion genes (PFGs) (FIG. 1a). On average each PFG have 1.44 fusion transcript isoforms. This suggests that PFGs are similar to annotated genes, which have complex alternatively-spliced isoforms. FIG. 4a shows that 11,488 PFGs have 5705 unique 5 ‘-genes and 5606 unique 3’-genes, respectively, which indicate that each 5′ or 3′ gene could form two different PFGs (FIG. 4a). The total 11488 PFGs have 8229 unique genes, 39% of which are involved in both 5′ and 3′ gene fusion (FIG. 4a). These data are consistent with previous findings that fusion events are recurrent in cancer. To evaluate origins of the fusion transcripts, we have analyzed distributions of the fusion transcripts among 39 cell lines. The numbers of fusion transcripts identified range from 21 in U2OS to 3374 in K562, lymphoblast of chronic myelogenous leukemia (FIG. 4b). Even though larger data result in more numbers of fusion transcripts, there is no direct correlation among them. Among eight cell lines that have >1,000 million RNA-seq reads, A549, adenocarcinomic human lung epithelial cells, have 1.06 numbers of splice sites per million reads (NSJMR) while MCF-7 and SK-N-SH have 0.33 and 0.38 NSJMR, which may partly reflect characteristics of cancer types.

To systematically characterize properties of these ECD39 fusion transcripts, we have arbitrarily classified these fusion events into five groups based on locations, orientations and distances between two genes: inter-chromosomal translocations, intra-chromosomal translocations, inversions, deletions, and read-through. These five genetic types of the fusion transcripts are defined as below. If 5′ and 3′ regions of a fusion transcript originate from two different chromosomes, this fusion transcript is thought to be inter-chromosomal translocation. If 5′ and 3′ regions of a fusion transcript are from the same chromosome and the distances between two regions are more than 1 million by in length, this fusion transcript is defined as the intra-chromosomal translocation. If 5′ and 3′ regions of a fusion transcript come from the same chromosome and the distances between two regions are larger than 1 million by in length and the both 5′ and 3′ regions are on the same strands, this fusion transcript is defined as the deletion. If 5′ and 3′ regions of a fusion transcript come from the same chromosome and the distances between two regions are less than 1 million by in length but 5′ and 3′ regions are the opposite strands, this fusion transcript is an inversion. If 5′ and 3′ regions of a fusion transcript come from the same chromosome and the distances between two regions are less than 1 million by in length and 5′ and 3′ regions are the same strands, this fusion transcript is thought to be read-through.

FIG. 4c shows that inter-chromosomal transcripts and FPGs are the highest among the five groups and accounted for 40% and 51%, while the deletion transcripts and PFGs are the lowest and count for 4.6% and 4.1% respectively. As Table 2 shows, inter-chromosomal translocation, intra-chromosomal translocation and deletion transcripts, whose gaps between two genes are ≧1 million bp, have very low fusion transcripts per PFG and ranged from 1.13 to 1.31. On the other hand, FIG. 4c has shown that the read-through and inversion transcripts, whose gaps between two genes are ≦1 million bp, have the most fusion transcripts per PFG, which are 2.22 and 1.86, respectively. That the fusion transcripts per PFG of read-though and inversion are much larger than those of inter-chromosomal translocation, intra-chromosomal translocation and deletion suggests that numbers of transcripts per PFG are associated with the gap sizes between two genes. Since the read-through genes are more like traditional genes, inter-chromosomal, intra-chromosomal and deletion fusion genes may have some mechanisms different from the “traditional” ones to generate fusion transcripts. Because identification of recurrent fusion transcripts among different types of cancer is extremely important for cancer diagnosis, therapy and prognosis, we have analyzed the recurrent fusion transcripts among the different groups of cancer cell lines.

To characterize the differences between the splicingcode method and other methods to identify fusion transcripts, we use the human multiple cancer types dataset (named as HMCT) from Stanford University (Giacomini, et al.). The HMCT dataset has seven samples, which have been generated by two types of sequence machines: Illuminia HiSeq 2000 and Genome Analyzer II. The four samples analyzed by Genome Analyzer II have 35 bp RNA-seq reads in length and three samples by Illuminia HiSeq 2000 have 100 bp RNA-seq reads. Due to short sequences lacking specificities, we have to discard four samples with shorter 35 bp sequences from further analysis. We have performed data analysis of three samples by Illuminia HiSeq 2000 and have identified 2205 fusion transcripts, four of which have been validated by Giacomini et al (Giacomini, et al. 2013).

Compared to other methods, we have less copy numbers of supporting RNA-seq reads per fusion transcript. We have analyzed the numbers of supporting sequence reads. Table 2 shows differences of supported sequence reads among the four genes uncovered by splicingcodes and validated by Giacomini et al (Giacomini, et al. 2013). From Table 2, the four genes have an average of the HMCT 54.7 sequence reads while they are supported by 7.5 sequence reads in our splicingcodes model, which are 7.5 folds less than the former. Table 2 shows that the BCL6|RAF1 fusion transcript has been supported by 39 HMCT reads and 2 SplicingCodes reads, respectively. This is almost 20 fold differences. This has demonstrated that splicingcodes model has used

TABLE 2
Differences of numbers of supported reads

| 5′ Genes | 3′ Genes | HMCT | SplicingCodes |
|---|---|---|---|
| BCL6 | RAF1 | 39 | 2 |
| FAM133B | CDK6 | 30 | 10 |
| EWSR1 | CREM | 120 | 14 |
| ABL1 | CBFB | 30 | 4 |

| | | |
|---|---|---|
| Average | 54.75 | 7.5 |

much stringent conditions.

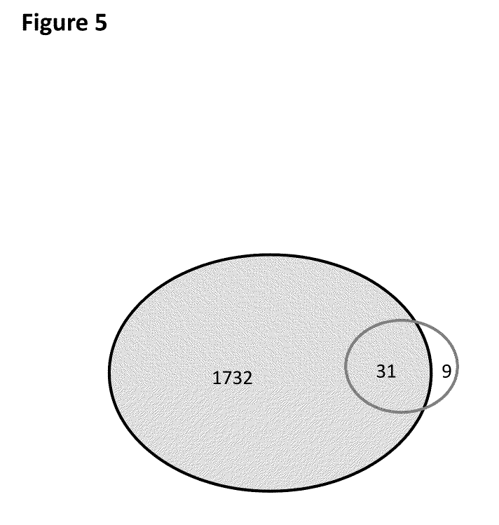

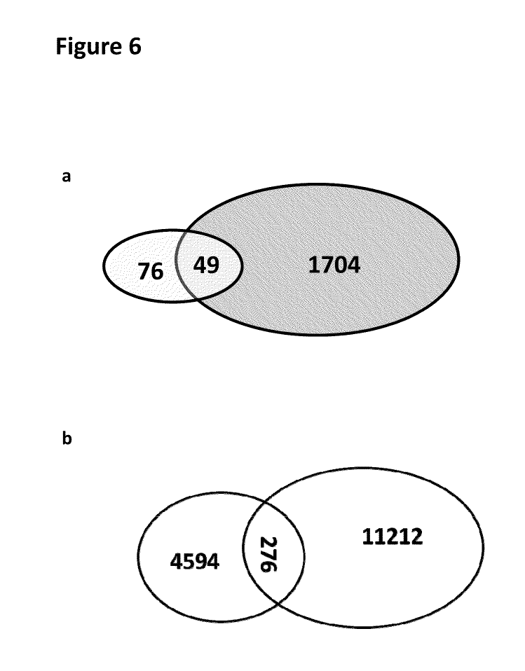

As shown in Table 1 and FIG. 4b, we have identified 2315 fusion transcripts with unique canonical splice sites, which represent 1763 unique putative fusion genes. Since MCF7 has been well-studied in transcriptional studies, it is natural for the MCF7 fusion transcripts from two different studies should have common identical fusion transcripts. Sakarya et al. have used a suffix array algorithm to analyze a MCF7 RNA-seq dataset and identified 40 and validated novel fusion genes (Sakarya, et al. 2012). FIG. 5 has shown the Van diagram between our fusion transcripts and those identified and validated by Sakarya et al (Sakarya, et al. 2012). Even though our datasets contain no MCF-7 RNA-seq datasets used by Sakarya et al., we have found that 31 (75%) of fusion transcripts are identical with those identified by Sakarya et al. (Sakarya, et al. 2012).

To further evaluate the quality of our fusion gene detection method, we have performed analysis on our ECD39 MCF7 fusion transcripts, which have MCF-7 2315 fusion transcripts representing 1763 fusion genes. Then, we parse out 132 GCD MCF7 fusion transcripts from the GCD datasets (Klijn, et al. 2015). FIG. 6a has shown that the ECD39's MCF7 fusion transcripts have been shown to have 49 (39.9%) genes overlapped with GCD MCF-7 132 fusion genes. Based on numbers of supporting reads, we can conclude that the fusion transcripts majorities of which are highly expressed. This strongly supports that our method is highly accurate.

To further characterize fusion transcripts, we have compared our data with large scale identification of 5451 fusion transcripts from 675 human cancer cell lines by Klijin et al. (referred as Genetech Cancer Data (GCD)) (Klijn, et al. 2015). Compared to the total GCD fusion transcripts, FIG. 6b shows that our ECD39 fusion transcripts have been found to have only identified 276 fusion transcripts, whose gene IDs are overlapped with GCD fusion genes, which count for 1.7%. Since the GCD fusion transcripts originated from 675 human cancer cell lines (Klijn, et al. 2015), there are eight cell lines overlapped between two datasets. Only small numbers of overlapped transcripts between two datasets of fusion transcripts have further confirmed that cancer is heterogeneous.

Reviewing identical fusion transcripts have shown that these fusion genes have been highly expressed based on the numbers of supporting sequence reads. It seems that all methods of identification of fusion transcripts are able to identify the highly-expressed fusion transcripts. However, our method identifies highly-expressed fusion transcripts, but also very lowly-expressed fusion transcripts.

In FIG. 4b, we have classified the fusion transcripts based on the ECD39 cell line types. Table 3 shows lists of the top ten fusion transcripts of the thirty-nine cancer cell lines.

TABLE 3
The top ten highly-expressed fusion transcripts in each of the
ECD39 thirty-nine cancer cell lines. Underlined gene symbols
represent a transcriptional unit of multiple gene complexes.

| | | | |
|---|---|---|---|
| Cell Lines | 5 Gene | 3 Gene | Counts |

Table 3a Top ten highly-expressed fusion
transcripts of A172, A375, A431 and A562

| | | | |
|---|---|---|---|
| A172 | CNOT1 | ARHGAP17 | 137 |
| A172 | SNTB2andVPS4A | IL34 | 130 |
| A172 | NSD1 | DHX15 | 85 |
| A172 | PIKFYVE | ACTL6A | 77 |
| A172 | URB1 | SLC27A1 | 70 |
| A172 | SMC4 | TAF9 | 69 |
| A172 | ABL1 | CBFB | 64 |
| A172 | DUSP14 | DDX52 | 60 |
| A172 | ALPK2 | ARID4BandRBM34 | 56 |
| A172 | METTL9 | SDK1 | 52 |
| A375 | KIAA1267 | ARL17AandARL17B | 60 |
| A375 | ST3GAL2 | COG4 | 48 |
| A375 | ALDH1A3 | CALM2andC2orf61 | 36 |
| A375 | HIF1AandSNAPC1 | PRKCH | 31 |
| A375 | ETV5 | TRA2B | 28 |
| A375 | C5orf30 | SYNCRIP | 26 |
| A375 | TPM4 | SUN1andGET4 | 25 |
| A375 | PPP3CA | HDGFRP3 | 24 |
| A375 | BAGE | BAGE3_{—} | 24 |
| A375 | MAP2K5 | SKOR1andPIAS1 | 23 |
| A431 | TPX2 | C20orf112 | 24 |
| A431 | PRIM1 | NACA | 21 |
| A431 | ZNF782 | ZNF510 | 19 |
| A431 | EGFR | PPARGC1A | 14 |
| A431 | LOC283299 | OVCH2 | 10 |
| A431 | EXOC4 | CHCHD3 | 10 |
| A431 | NRIP1 | LOC100128341 | 8 |
| A431 | SLC38A1 | SRSF2IP | 8 |
| A431 | FAM18B2andCDRT4 | TEKT3 | 8 |
| A431 | CLTC | TMEM49 | 8 |
| A549 | MFGE8 | HAPLN3 | 468 |
| A549 | SCAMP2 | WDR72 | 411 |
| A549 | KIAA1267 | ARL17AandARL17B | 212 |
| A549 | C19orf47 | AKT2 | 139 |
| A549 | UBA2 | WTIP | 133 |
| A549 | P2RY6 | ARHGEF17 | 112 |
| A549 | NCEH1 | MUC13 | 78 |
| A549 | ACCS | EXT2 | 73 |
| A549 | MFGE8 | HAPLN3 | 64 |
| A549 | ST6GALNAC4 | ST6GALNAC6andAK1 | 53 |

Table 3b Top ten highly-expressed fusion
transcripts of CUTLL, Caki2, Daoy and G401

| | | | |
|---|---|---|---|
| CUTLL | TRBV_{—} | NOTCH1 | 534 |
| CUTLL | LZTFL1 | SLC6A20 | 200 |
| CUTLL | THEMIS | PTPRK | 41 |
| CUTLL | C6orf106 | LOC100132288 | 34 |
| CUTLL | SLC35A3 | HIAT1 | 32 |
| CUTLL | TRBV_{—} | NOTCH1 | 30 |
| CUTLL | UBA2 | WTIP | 24 |
| CUTLL | ZNF782 | ZNF510 | 24 |
| CUTLL | ERBB2IP | SFRS12 | 19 |
| CUTLL | PSMA4 | CHRNA5 | 17 |
| Caki2 | MICALL1 | POLR2F | 524 |
| Caki2 | PKD1 | NTHL1 | 201 |
| Caki2 | DLG5 | TPH1andSERGEF | 158 |
| Caki2 | TSSC1 | KIDINS220 | 145 |
| Caki2 | TUSC3 | EXOC6B | 135 |
| Caki2 | PCMT1 | PDSS2 | 127 |
| Caki2 | MED26_{—} | ZBTB1 | 115 |
| Caki2 | C6orf105 | ZCCHC11 | 103 |
| Caki2 | CELSR1 | TMCO3 | 82 |
| Caki2 | AGPS | VAPA | 76 |
| Daoy | TM7SF3 | C12orf11 | 164 |
| Daoy | KIF5B | ZEB1 | 132 |
| Daoy | ALCAM | ACTR3 | 69 |
| Daoy | GNB2L1_{—} | ADPRHL2 | 65 |
| Daoy | ZNF782 | ZNF510 | 64 |
| Daoy | RC3H2 | KATNA1 | 62 |
| Daoy | YIPF4 | DYM | 60 |
| Daoy | G3BP1 | ANXA2 | 58 |
| Daoy | LEPROTL1 | INTS9 | 56 |
| Daoy | FNBP1 | GTF2IRD2B | 53 |
| G401 | LOC283299 | OVCH2 | 74 |
| G401 | HRSP12 | GDI2 | 69 |
| G401 | CLN6andCALML4 | GABRA5 | 55 |
| G401 | MLL3 | BAGE3_{—} | 45 |
| G401 | MTHFD2 | MOBKL1B | 44 |
| G401 | GDPD5 | CHD8 | 38 |
| G401 | PRDX2 | GNAS | 37 |
| G401 | LOC728190 | GLUD1 | 36 |
| G401 | TBC1D30 | MSRB3 | 32 |
| G401 | DCUN1D2 | LAMP1 | 26 |

Table 3c Top ten highly-expressed fusion
transcripts of H4, H460, HCC1599 and HCT116

| | | | |
|---|---|---|---|
| LHCN-M2 | ZNF782 | ZNF510 | 91 |
| LHCN-M2 | EEF1DP3 | FRY | 69 |
| LHCN-M2 | TBC1D23 | NIT2 | 30 |
| LHCN-M2 | MICAL3 | BCL2L13 | 28 |
| LHCN-M2 | ADAM9 | ADAM32 | 27 |
| LHCN-M2 | ZBED5_{—} | KIAA0319L | 25 |
| LHCN-M2 | SLC7A5P2 | LOC641298 | 25 |
| LHCN-M2 | NRIP1 | LOC100128341 | 23 |
| LHCN-M2 | CTNNA1 | SIL1 | 21 |
| LHCN-M2 | WLS | DIRAS3 | 19 |
| LIM2405 | VAX2 | ATP6V1B1 | 5 |
| LIM2405 | SUMO2 | HN1 | 3 |
| LIM2405 | ACCS | EXT2 | 3 |
| LIM2405 | CHCHD2 | PHKG1 | 3 |
| LIM2405 | NRIP1 | LOC100128341 | 2 |
| LIM2405 | XK | CYBB | 2 |
| LIM2405 | ACCS | EXT2 | 2 |
| LIM2405 | SLC35A3 | HIAT1 | 2 |
| LIM2405 | XK | CYBB | 1 |
| LIM2405 | ZW10 | TMPRSS5 | 1 |
| LIM1899 | UHRF1BP1L | ANKS1B | 102 |
| LIM1899 | CDK13 | C7orf10 | 14 |
| LIM1899 | MIR17HG | GPC5 | 12 |
| LIM1899 | ARNTL | MICAL2 | 9 |
| LIM1899 | UHRF1BP1L | ANKS1B | 9 |
| LIM1899 | SLC35A3 | HIAT1 | 8 |
| LIM1899 | LOC389641 | CHMP7 | 7 |
| LIM1899 | ZNF619andZNF620 | ZNF621 | 6 |
| LIM1899 | PLEKHM1P | LOC146880 | 5 |
| LIM1899 | UBA2 | WTIP | 5 |
| M059J | SLC23A2 | RNF130 | 51 |
| M059J | NLGN1 | IFI6 | 46 |
| M059J | PRSS23 | PSMB2 | 41 |
| M059J | CPSF6 | ZNF532 | 35 |
| M059J | CYLD | PHKB | 34 |
| M059J | CCBL2 | MYO19 | 33 |
| M059J | KIAA1267 | ARL17AandARL17B | 31 |
| M059J | HDAC8 | CITED1 | 30 |
| M059J | SIKE1andCSDE1andNRAS | ZNF148andSLC12A8 | 29 |
| M059J | FLJ34690 | MYOCD | 26 |

Table 3d Top ten highly-expressed fusion
transcripts of HT1080, HT29, Karpas422 and KATOIII

| | | | |
|---|---|---|---|
| HT1080 | YWHAQ | LPIN1 | 36 |
| HT1080 | WDFY1andAP1S3 | SERPINE2 | 32 |
| HT1080 | FBXO34 | FOXN3 | 27 |
| HT1080 | GAS6 | TFDP1 | 26 |
| HT1080 | KCNH5 | HIF1AandSNAPC1 | 26 |
| HT1080 | UBE2S | ACTN4 | 25 |
| HT1080 | BRIX1 | DPY19L4 | 25 |
| HT1080 | DYNC1H1 | PPP2R5C | 25 |
| HT1080 | CALN1 | HS3ST3B1 | 24 |
| HT1080 | ECSITandZNF653 | PRKCSH | 23 |
| HT29 | MTMR3 | APOH | 488 |
| HT29 | USP6NL | UPF2 | 269 |
| HT29 | KIAA1267 | ARL17AandARL17B | 194 |
| HT29 | UBA2 | WTIP | 79 |
| HT29 | RPL23AP5 | NME4andDECR2 | 78 |
| HT29 | EEF1DP3 | FRY | 60 |
| HT29 | C11orf9 | FAM132A | 53 |
| HT29 | PAWR | NAP1L1 | 53 |
| HT29 | TRA2B | RABGAP1andGPR21 | 45 |
| HT29 | PAOXandMTG1 | LOC619207 | 44 |
| Karpas422 | KIAA1267 | ARL17AandARL17B | 271 |
| Karpas422 | HNRNPA1L2 | EXOC4 | 82 |
| Karpas422 | DCAF16andFAM184B | PHF14 | 66 |
| Karpas422 | LOC100288132 | TRMT1 | 41 |
| Karpas422 | RPL23AP5 | NME4andDECR2 | 40 |
| Karpas422 | PLEKHM1P | LOC146880 | 36 |
| Karpas422 | EPN1 | BPTF | 30 |
| Karpas422 | MKNK2 | AGXT2L2 | 27 |
| Karpas422 | TRA2A | IGF2BP3 | 27 |
| Karpas422 | CDKL3andPPP2CA | SKP1 | 26 |
| KATOIII | PAFAH1B2 | SIK3 | 60 |
| KATOIII | FGFR2 | ULK4 | 18 |
| KATOIII | FOXA2 | NCRNA00261 | 7 |
| KATOIII | UBA2 | WTIP | 6 |
| KATOIII | CECR7andIL17RA | LOC100132288 | 3 |
| KATOIII | NRIP1 | LOC100128341 | 3 |
| KATOIII | ZNF782 | ZNF510 | 3 |
| KATOIII | HDAC4 | ILKAP | 3 |
| KATOIII | PLEKHM1P | LOC146880 | 3 |
| KATOIII | CTNNB1 | ULK4 | 3 |

Table 3e Top ten highly-expressed fusion
transcripts of LHCN-M2, LIM2405, LIM1899 and M059J.

| | | | |
|---|---|---|---|
| LHCN-M2 | ZNF782 | ZNF510 | 91 |
| LHCN-M2 | EEF1DP3 | FRY | 69 |
| LHCN-M2 | TBC1D23 | NIT2 | 30 |
| LHCN-M2 | MICAL3 | BCL2L13 | 28 |
| LHCN-M2 | ADAM9 | ADAM32 | 27 |
| LHCN-M2 | ZBED5_{—} | KIAA0319L | 25 |
| LHCN-M2 | SLC7A5P2 | LOC641298 | 25 |
| LHCN-M2 | NRIP1 | LOC100128341 | 23 |
| LHCN-M2 | CTNNA1 | SIL1 | 21 |
| LHCN-M2 | WLS | DIRAS3 | 19 |
| LIM2405 | VAX2 | ATP6V1B1 | 5 |
| LIM2405 | SUMO2 | HN1 | 3 |
| LIM2405 | ACCS | EXT2 | 3 |
| LIM2405 | CHCHD2 | PHKG1 | 3 |
| LIM2405 | NRIP1 | LOC100128341 | 2 |
| LIM2405 | XK | CYBB | 2 |
| LIM2405 | ACCS | EXT2 | 2 |
| LIM2405 | SLC35A3 | HIAT1 | 2 |
| LIM2405 | XK | CYBB | 1 |
| LIM2405 | ZW10 | TMPRSS5 | 1 |
| LIM1899 | UHRF1BP1L | ANKS1B | 102 |
| LIM1899 | CDK13 | C7orf10 | 14 |
| LIM1899 | MIR17HG | GPC5 | 12 |
| LIM1899 | ARNTL | MICAL2 | 9 |
| LIM1899 | UHRF1BP1L | ANKS1B | 9 |
| LIM1899 | SLC35A3 | HIAT1 | 8 |
| LIM1899 | LOC389641 | CHMP7 | 7 |
| LIM1899 | ZNF619andZNF620 | ZNF621 | 6 |
| LIM1899 | PLEKHM1P | LOC146880 | 5 |
| LIM1899 | UBA2 | WTIP | 5 |
| M059J | SLC23A2 | RNF130 | 51 |
| M059J | NLGN1 | IFI6 | 46 |
| M059J | PRSS23 | PSMB2 | 41 |
| M059J | CPSF6 | ZNF532 | 35 |
| M059J | CYLD | PHKB | 34 |
| M059J | CCBL2 | MYO19 | 33 |
| M059J | KIAA1267 | ARL17AandARL17B | 31 |
| M059J | HDAC8 | CITED1 | 30 |
| M059J | SIKE1andCSDE1andNRAS | ZNF148andSLC12A8 | 29 |
| M059J | FLJ34690 | MYOCD | 26 |

Table 3f Top ten highly-expressed fusion
transcripts of MDA-MB-231, MG63, OCI-Ly7 and PC3

| | | | |
|---|---|---|---|
| MDA-MB-231 | SLC29A1 | HSP90AB1 | 19 |
| MDA-MB-231 | REV1 | SUPT3H | 13 |
| MDA-MB-231 | HARS | TTC27 | 7 |
| MDA-MB-231 | SLC35A3 | HIAT1 | 5 |
| MDA-MB-231 | LOC283299 | OVCH2 | 4 |
| MDA-MB-231 | HARS | TTC27 | 4 |
| MDA-MB-231 | SLC35A3 | HIAT1 | 4 |
| MDA-MB-231 | PDPK2 | TCEB2 | 4 |
| MDA-MB-231 | TRIB3 | RBCK1 | 3 |
| MDA-MB-231 | RPS6KB1 | TMEM49 | 3 |
| MG63 | TFG | GPR128 | 495 |
| MG63 | DNER | ELL2 | 136 |
| MG63 | MTAPandCDKN2BAS | BNC2 | 121 |
| MG63 | THSD4 | LRRC49 | 108 |
| MG63 | PSMD8 | ATF7andNPFF | 107 |
| MG63 | ELL2 | TRIP12 | 78 |
| MG63 | HEATR7A | PARP10 | 76 |
| MG63 | TP53 | VAV1 | 64 |
| MG63 | CLIP4 | EPHB4 | 63 |
| MG63 | GAS6 | COMMD3andBMI1 | 57 |
| OCI-Ly7 | IGL_{—} | GRAPL | 69 |
| OCI-Ly7 | PIK3C2_{—} | UBE2D2 | 41 |
| OCI-Ly7 | SIT1 | CD72 | 38 |
| OCI-Ly7 | HIPK2 | NDUFA5 | 38 |
| OCI-Ly7 | ZC3HAV1 | UBN2 | 34 |
| OCI-Ly7 | LOC389641 | CHMP7 | 33 |
| OCI-Ly7 | UBA2 | WTIP | 30 |
| OCI-Ly7 | AVL9 | SP4 | 28 |
| OCI-Ly7 | PPFIA1 | RTN3 | 27 |
| OCI-Ly7 | DNAJB1 | PKM2 | 24 |
| PC3 | C12orf51 | RPL6 | 53 |
| PC3 | SAMD8 | ADKandMRPL35P3 | 41 |
| PC3 | PAAF1 | RPL141 | 36 |
| PC3 | AGAP6 | FRMPD2 | 29 |
| PC3 | ZMIZ1 | CTNNA3 | 26 |
| PC3 | FAF1 | AGBL4 | 25 |
| PC3 | MPP5 | FUT8 | 25 |
| PC3 | C1orf55 | ENAH | 25 |
| PC3 | MAD1L1 | CHFRandGOLGA3 | 24 |
| PC3 | PTK2 | SKP1 | 24 |

Table 3g Top ten highly-expressed fusion
transcripts of RPMI-7951, SJCRH30, SK-Mel-5 and SK-N-DZ

| | | | |
|---|---|---|---|
| RPMI-7951 | RPS5P1andDSE | FAM26F | 26 |
| RPMI-7951 | RPS11andSNORD35B | LUC7L | 23 |
| RPMI-7951 | MYO19 | ZNHIT3 | 22 |
| RPMI-7951 | LHFP | CREBZF | 22 |
| RPMI-7951 | EEF1DP3 | FRY | 22 |
| RPMI-7951 | ZNF649andZNF577 | TATDN2andGHRLOS | 22 |
| RPMI-7951 | UTRN | NME7 | 20 |
| RPMI-7951 | TRIO | CYBRD1 | 19 |
| RPMI-7951 | CRTC3 | MAPKBP1 | 19 |
| RPMI-7951 | TOPORSandDDX58 | ACO1 | 19 |
| SJCRH30 | MARS | AVIL | 714 |
| SJCRH30 | PAX3 | FOXO1 | 283 |
| SJCRH30 | SNORD114-1 | MEG8- | 135 |
| SJCRH30 | MEGF11 | RPL9P25andTIPIN | 44 |
| SJCRH30 | RPS6KC1 | FLVCR1 | 40 |
| SJCRH30 | FANCD2 | MTHFD1L | 32 |
| SJCRH30 | THSD4 | SERHL2 | 27 |
| SJCRH30 | ZNF782 | ZNF510 | 26 |
| SJCRH30 | NRIP1 | LOC100128341 | 23 |
| SJCRH30 | RAD18 | OXTR | 23 |
| SK-Mel-5 | C1orf43 | SCAMP3 | 1067 |
| SK-Mel-5 | UBE2Q1 | VPS72andTMOD4 | 279 |
| SK-Mel-5 | FSTL5 | MRPL21 | 65 |
| SK-Mel-5 | LOC340357 | LONRF1 | 27 |
| SK-Mel-5 | ZNF782 | ZNF510 | 27 |
| SK-Mel-5 | C1orf43 | SCAMP3 | 21 |
| SK-Mel-5 | CTTN | TRIM37 | 20 |
| SK-Mel-5 | DIXDC1 | SDHD | 17 |
| SK-Mel-5 | TUFT1 | EFNA4andEFNA3 | 17 |
| SK-Mel-5 | LOC729082 | RGS20 | 17 |
| SK-N-DZ | MAP1D | FARSB | 1327 |
| SK-N-DZ | KIAA1267 | ARL17AandARL17B | 682 |
| SK-N-DZ | CTSC | MAML2 | 134 |
| SK-N-DZ | DBI | SPAG16 | 66 |
| SK-N-DZ | AACSL | ZNF354A | 57 |
| SK-N-DZ | C2orf43 | FLJ30838 | 57 |
| SK-N-DZ | KLK4 | KLKP1 | 55 |
| SK-N-DZ | PSMB7 | CKS2 | 55 |
| SK-N-DZ | CAPZA2 | PTTG1 | 54 |
| SK-N-DZ | SNORD114-1 | MEG8 | 53 |

Table 3h Top ten highly-expressed fusion
transcripts of SK-N-SH, SUN-16, Hela-3 and REC1.

| | | | |
|---|---|---|---|
| SK-N-SH | EXOC4 | PVT1 | 235 |
| SK-N-SH | C19orf47 | AKT2 | 225 |
| SK-N-SH | EXOC4 | PVT1 | 142 |
| SK-N-SH | PAOXandMTG1 | LOC619207 | 131 |
| SK-N-SH | ACCS | EXT2 | 116 |
| SK-N-SH | VAX2 | ATP6V1B1 | 111 |
| SK-N-SH | PVT1 | EXOC4 | 107 |
| SK-N-SH | LMAN2 | MXD3andRAB24 | 93 |
| SK-N-SH | MFGE8 | HAPLN3 | 87 |
| SK-N-SH | RPL23AP5 | NME4andDECR2 | 84 |
| SUN16 | PVT1 | SLC1A2 | 22 |
| SUN16 | PVT1 | SLC1A2 | 14 |
| SUN16 | LOC389641 | CHMP7 | 4 |
| SUN16 | STS | VCX | 4 |
| SUN16 | EEF1DP3 | FRY | 4 |
| SUN16 | CTNNBIP1 | CLSTN1 | 4 |
| SUN16 | CENPK | UVRAG | 4 |
| SUN16 | NDUFAF2 | ZSWIM6 | 4 |
| SUN16 | RND3 | RALB | 4 |
| SUN16 | CMIP | DYNLRB2 | 4 |
| Hela-3 | RPS6KB1 | TMEM49 | 256 |
| Hela-3 | HNRNPUL2andBSCL2 | C11orf49 | 253 |
| Hela-3 | GNB1 | NADK | 149 |
| Hela-3 | ST6GALNAC4 | ST6GALNAC6andAK1 | 124 |
| Hela-3 | KIAA1267 | ARL17AandARL17B | 120 |
| Hela-3 | CCDC123 | PEPD | 79 |
| Hela-3 | UBA2 | WTIP | 64 |
| Hela-3 | PAOXandMTG1 | LOC619207 | 49 |
| Hela-3 | GNB1 | NADK | 47 |
| Hela-3 | C19orf47 | AKT2 | 42 |
| REC1 | AKNA | FBXL20 | 98 |
| REC1 | FBXL20 | AKNA | 41 |
| REC1 | MYST3 | PLEKHA5 | 27 |
| REC1 | LOC619207 | CYP2E1 | 20 |
| REC1 | FCRL2 | FCRL3 | 18 |
| REC1 | SLC29A1 | HSP90AB1 | 18 |
| REC1 | ZNF782 | ZNF510 | 14 |
| REC1 | LOC285972 | GIMAP8 | 13 |
| REC1 | PAOXandMTG1 | LOC619207 | 12 |
| REC1 | ST6GALNAC4 | ST6GALNAC6andAK1 | 11 |

Table 3i Top ten highly-expressed fusion
transcripts of U87, U2OS, U251 and MCF7.

| | | | |
|---|---|---|---|
| U87 | BMP7 | TMPRSS15 | 10 |
| U87 | PPP1R13L | ZNF541 | 9 |
| U87 | PPP1R13L | ZNF541 | 5 |
| U87 | ZNF782 | ZNF510 | 5 |
| U87 | CDKL3andPPP2CA | SKP1 | 5 |
| U87 | BMP7 | TMPRSS15 | 4 |
| U87 | UBA2 | WTIP | 3 |
| U87 | SACS | SGCG | 3 |
| U87 | C15orf26 | IL16 | 2 |
| U87 | ATP2C1 | NEK11 | 2 |
| U2OS | ADAM9 | ADAM32 | 33 |
| U2OS | UBA2 | WTIP | 6 |
| U2OS | SLC35A3 | HIAT1 | 4 |
| U2OS | MRPS10 | GUCA1B | 3 |
| U2OS | HDAC8 | CITED1 | 3 |
| U2OS | BAG4 | DDHD2 | 2 |
| U2OS | BAG4 | DDHD2 | 1 |
| U251 | ATP11C | MCF2 | 49 |
| U251 | NRIP1 | LOC100128341 | 6 |
| U251 | RASSF8 | SSPN | 4 |
| U251 | RAB31 | TXNDC2 | 4 |
| U251 | LARP1 | CNOT8 | 4 |
| U251 | ARF3 | FKBP11 | 3 |
| U251 | CORO1C | SELPLG | 3 |
| U251 | PPP1R12A | PAWR | 3 |
| MCF7 | ARFGEF2 | SULF2 | 2176 |
| MCF7 | RPS6KB1 | TMEM49 | 2107 |
| MCF7 | TANC2 | CA4 | 1526 |
| MCF7 | RPS6KB1 | TMEM49 | 1502 |
| MCF7 | PAPOLA | AK7 | 873 |
| MCF7 | SYTL2 | PICALM | 764 |
| MCF7 | ADAMTS19 | SLC27A6 | 685 |
| MCF7 | RPS6KB1 | DIAPH3 | 597 |
| MCF7 | ABCA5 | PPP4R1L | 535 |
| MCF7 | DEPDC1B | ELOVL7 | 532 |

Table 3j Top ten highly-expressed fusion
transcripts of HepG2, K562 and SJSA1.

| | | | |
|---|---|---|---|
| HepG2 | AHSG | GYG2P1andARSFP1 | 308 |
| HepG2 | FOXA2 | NCRNA00261 | 252 |
| HepG2 | ZNF782 | ZNF510 | 142 |
| HepG2 | LMO7 | UCHL3 | 134 |
| HepG2 | LMAN2 | MXD3andRAB24 | 90 |
| HepG2 | NRIP1 | LOC100128341 | 78 |
| HepG2 | PAOXandMTG1 | LOC619207 | 71 |
| HepG2 | VAX2 | ATP6V1B1 | 70 |
| HepG2 | SLC29A1 | HSP90AB1 | 58 |
| HepG2 | NRIP1 | LOC100128341 | 58 |
| K562 | BCR | ABL1 | 4043 |
| K562 | BAT3 | SLC44A4 | 2760 |
| K562 | NUP214 | XKR3 | 2443 |
| K562 | KIAA1267 | ARL17AandARL17B | 781 |
| K562 | C10orf76 | KCNIP2andMGEA5 | 432 |
| K562 | IMMP2L | DOCK4 | 254 |
| K562 | C15orf26 | IL16 | 218 |
| K562 | C16orf87 | ORC6L | 202 |
| K562 | PRIM1 | NACA | 188 |
| K562 | BAT3 | SLC44A4 | 171 |
| SJSA1 | HMGA2 | LUM | 4210 |
| SJSA1 | ARHGEF7 | CPM | 281 |
| SJSA1 | SNORD114-1 | MEG8 | 113 |
| SJSA1 | SLC7A5 | BANP | 107 |
| SJSA1 | SP140L | LCA5 | 96 |
| SJSA1 | KIF5A | STK24 | 94 |
| SJSA1 | KPNA6 | UBAP2L | 69 |
| SJSA1 | KIAA0427 | SMAD2 | 57 |
| SJSA1 | AGRN | TMEM8A | 51 |
| SJSA1 | SLC12A2 | DDX5 | 51 |

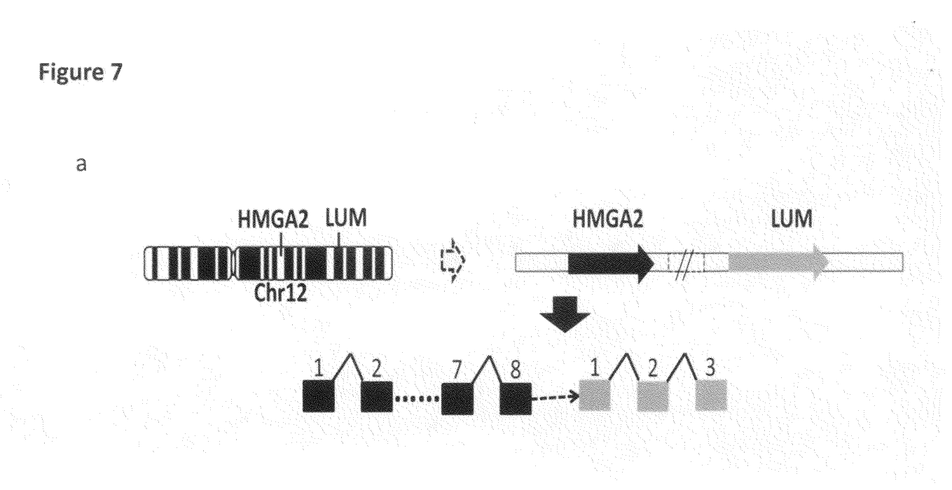

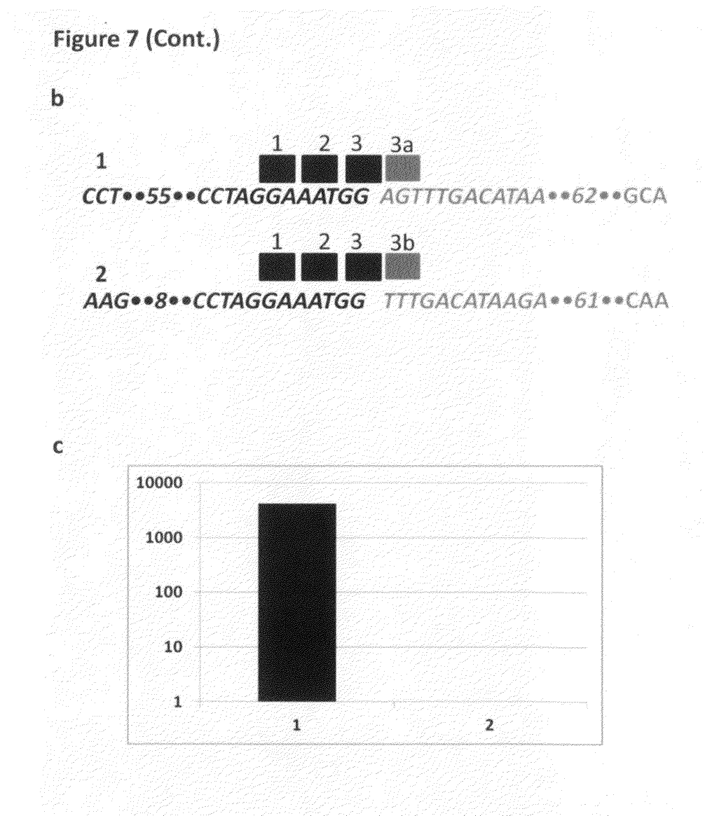

To characterize these large numbers of fusion transcripts, we have analyzed the fusion transcripts based on cancer cell lines and their supporting sequence reads. Table 3 has shown that many fusion transcripts are expressed at very high levels. However, they are often detected only in one type of cancer and are not recurrent in other cancer types. One of the most highly-expressed putative fusion genes is HMGA2-LUM putative fusion gene in osteosarcoma SJSA1 cell, which is a putative fusion gene between HMGA2 gene, encoding high mobility group AT-hook2 and associated with mesenchymoma and LUM gene coding for lumican and associated with corneal dystrophy. FIG. 7a shows that HMGA2 and LUM genes have undergone potential intra-chromosomal translocations and they are brought to close each other on the chromosome 12. FIG. 7b shows that HMGA2-LUM fusion gene has two isoforms (Isoform 1 and Isoform 2). FIG. 7b shows that Isoform 1 and Isoform 2 differ by only two nucleotides at their fusion junctions. Isoform 1 will have the normal LUM's last exon and generate a HMGA2-LUM fusion protein. On the hand, Isoform 2 will result in a truncated HMGA2 protein, which is 50 amino acids shorter than the Isoform 1. FIG. 7c shows that two expression levels differ by 4200 folds. The fact that HMGA2 isoforms similar to Isoform 2 have been observed in normal human tissues and cells has suggested that the Isoform 1 fusion protein may play in important role in SJSA1 cancer development.

As discussed above, we have adopted much stringent conditions to identify fusion transcripts. As shown in Table 2, our supporting sequence reads are 7.5 folds less than others. Technically, it is much more difficult for us to experimentally verify the lowly-expressed fusion transcripts than those highly-expressed fusion transcripts. Since we have identified large numbers of fusion transcripts, it is not practical for us to use “traditional” RT-PCR approaches and other “traditional” methods to validate these large numbers of fusion transcripts. However, if we can use the traditional RT-PCR methods to validate some lowly-expressed fusion transcripts, it will greatly help us to understand the characteristics of these fusion transcripts and will lay solid foundations for large-scale verification of all fusion transcripts, such as RNA CaptureS eq (Mercer, et al. 2014).

To verify the lowly-expressed fusion transcripts, we have isolated total RNAs from cancer cell lines MCF-7, Hela-3, HepG2, BT-474, K562, 293T and other cancer cell lines while GM12878 and MCF-10A normal cell line have been used as controls. Total RNAs are isolated by Qiagen RNeasy mini columns with DNase I digestion as suggested by the manufacturer. Briefly, 1×10⁶ cultured cells are harvested by centrifuging for 5 min at 300×g. Supernatants are removed by aspiration. Cell pellets are disrupted for 30 seconds in 350 μl of Buffer RLT. The lysates are pipetted directly into a QIAshredder spin column placed in a 2 ml collection tube, and centrifuge for 2 min at full speed. One volume of 70% ethanol is added to the cleared lysate, and mix well by pipetting. 700 μl of the sample are transferred to RNeasy mini spin columns sitting in a 2-ml collection tube and the columns are centrifuged for 30 seconds at maximum speed and flow-through is discarded. 700 μl Buffer RW1 are added onto the RNeasy column, the RNeasy columns are centrifuged for 30 seconds at maximum speed and flow-through is discarded. 350 μl Buffer RWT are added into the RNeasy Mini spin column and centrifuge for 15 at 8000×g. To remove potential DNA contamination, after 10 μl DNase I stock solution is mixed with 70 μl Buffer RDD by gently inverting tubes, the DNase solution is added into the RNeasy columns and incubated at room temperature for 30 minutes. The columns are washed again by adding 350 μl Buffer RWT. After RNeasy columns are transferred to new 2-ml collection tubes, the columns are washed twice using 500 μl Buffer RPE by centrifuging for 30 seconds at maximum speed. RNAs are eluted from the columns by adding 30 μl of RNase-free water

The first-strand cDNA synthesis is carried out using oligo(T)15 and/or random hexamers by TaqMan Reverse Transcription Reagents (Applied Biosystems Inc., Foster City, Calif., USA) as suggested by the manufacturer. In brief, to prepare the 2×RT master mix, we pool 10 μl of reaction mixes containing final concentrations of 1×RT Buffer, 1.75 mM MgCl₂, 2 mM dNTP mix (0.5 mM each), 5 mM DTT, 1× random primers, 1.0 U/μl RNase inhibitor and 5.0 U/μl MultiScribe®. The master mixes are prepared, spanned down and placed on ice. 10 μl of 2×RNA mixes containing 2 ug of total RNA are added into 10 μl 2× master mixes and mixed well. The reaction mixes are then placed in a thermal cycler of 25° C., 10 min, 37° C. 120 min, 95° C., 5 min and 4° C., ∞. The resulted cDNAs are diluted by 80 μl of H₂O.

To identify novel human fusion transcripts, fusion transcript specific primers have been designed to cover the 5′ and 3′ fusion transcripts. The primers are designed using the primer-designing software (SDG 2015). 5 μl of the cDNAs generated above are used to amplify fusion transcripts by PCR. PCR amplifications are carried out by HiFi Taq polymerase (Invitrogen, Carlsbad, Calif., USA). PCR reactions have been carried out by HiFi Taq polymerase (Invitrogen, Carlsbad, Calif., USA) using cycles of 94° C., 15″, 60-68° C., 15″ and 68° C., 2-5 min. The PCR products are separated on 2% agarose gels. The expected products are excised from gels and cloned into pCR4.0 TA vector (Invitrogen, Carlsbad, Calif., USA). Fusion transcripts are then verified by blast and manual inspection.

As discussed above, many highly-expressed fusion transcripts have been successfully validated in different cancer datasets. In our approach, we have identified majorities of fusion transcripts are expressed at very low levels based on the numbers of supporting sequence reads. After we have performed RNA-seq analysis of different lymphoblastoid cell lines from different individuals, we have found that lowly-expressed fusion transcripts are shown to have strong individuality. That is, these fusion transcripts can be detected only in one lymphoblastoid cell line, but not in any of other lymphoblastoid cell lines. Later, experimental data have confirmed this conclusion. So we have selected numbers of lowly-expressed fusion transcripts for validation and we have validated six of them so far.

Table 4 shows the list of the validated fusion transcripts expressed at very

TABLE 4
Characteristics of some lowly-expressed fusion transcripts
validated by RT-PCR and following by DNA sequencing.

| Fusion Transcripts | Cell Types | NSJMR |
|---|---|---|
| GABBR1andUBD\|PSPH | BT-474 | 0.001 |
| ncRNA00188_\|GNAI3 | GM12878 | 0.00051 |
| LRRC37A3\|VNN2 | BT-474 | 0.000891 |
| CPSF6\|CACNA1E | GM12878 | 0.000455 |
| FAM164A\|RASA4PandPOLR2J4 | Heart | 0.000394 |
| RRP8\|RAB2A | Heart | 0.00095 |

low levels, which range from 3.94×10⁻⁴ to 1×10⁻³ numbers of splice junctions per million reads (NSJMR).

Table 5 shows the primers used to validate the fusion transcripts.

TABLE 5
List of primers for validation of fusion transcripts.

| Fusion Transcripts | 5′ Primers | 3′ Primers |
|---|---|---|
| GABBR1andUBD\|PSPH | TGAGTAGCTGAAACTACAGGATGCTT | TCAGTGATATACCATTTGGCGTT, |
| ncRNA00188_\|GNAI3 | CACAGTGGGGGTGTGCAAAC | CGAGACCGTGACCGAGAG |
| LRRC37A3\|VNN2 | TGAGTAGCTGGGATTGCAGTACCA | TCCGGCTTTTCAGGGACATTAA |
| CPSF6\|CACNA1E | CGAGACCGTGACCGAGAG | CGAGACCGTGACCGAGAG |
| FAM164A\|RASA4PandPOLR2I4 | CCTCCCCAACCAAGCTTTCTGTA | CCTTCAATGCCTTTAATATTTCCACC |
| RRP8\|RAB2A | GATGTTCGAACCTTTCTGCGG | ACGACCTTGTGATGGAACGAAA |

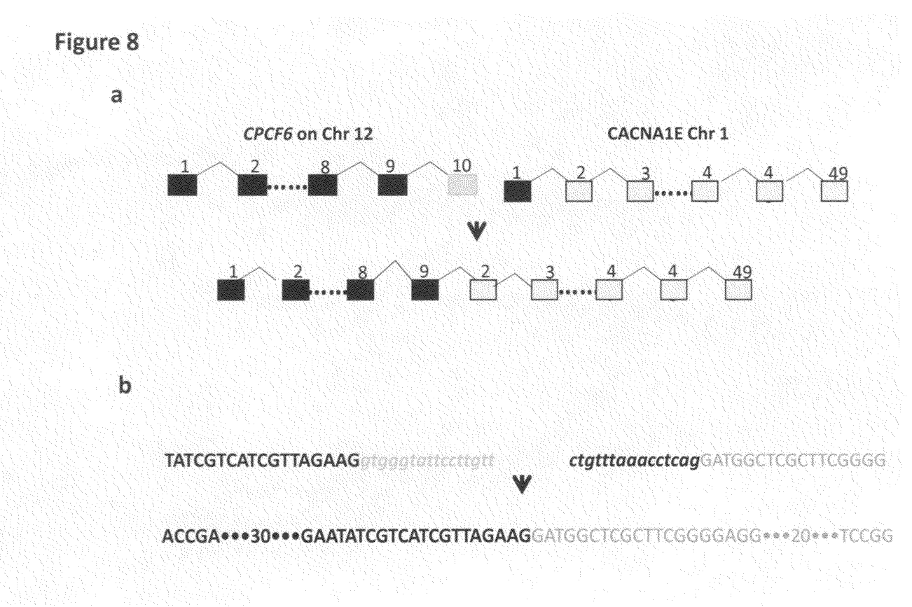

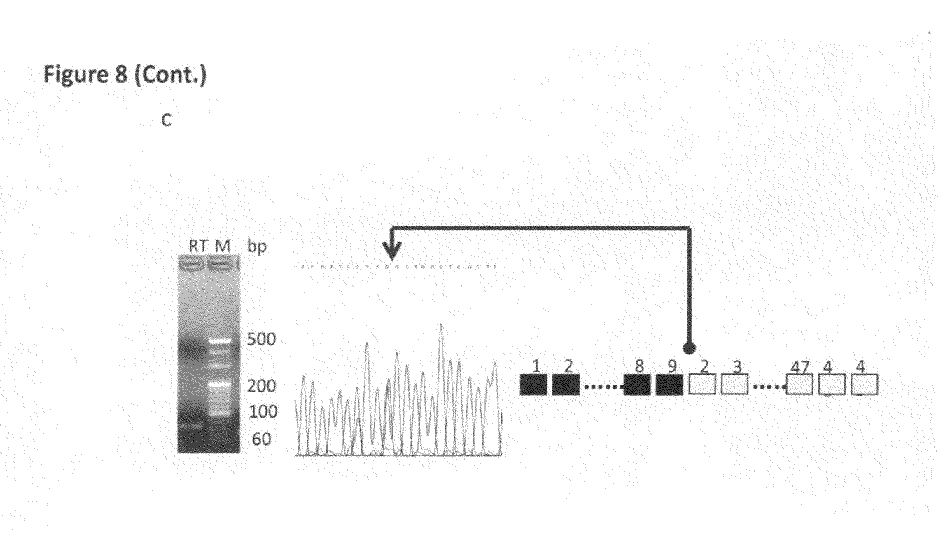

As shown in Table 4, the CPSF6|CACNA1E fusion transcripts have been found to be expressed at very low levels in the lymphoblastoid cell line of GM12878 and its NSJMR is 4.55×10⁻¹⁰. CPSF6 gene, encoding Cleavage And Polyadenylation Specific Factor 6, has been shown to be located on the chromosome 12 while CACNA1E gene, coding for Calcium Channel, R Type, Alpha-1 Polypeptide, is locate on the chromosome 1. The CPSF6|CACNA1E fusion transcripts are interchromosomal translocations. FIG. 8 has shown a schematic diagram of procedures to verify CPSF6|CACNA1E fusion transcripts in lymphoblastoid cell line. A potential translocation has brought CPSF6 and CACNA1E genes together. Total RNAs are isolated from the GM12878 cell lines and cDNAs are generated by TaqMan Reverse Transcription Reagents. Pair of primers has been designed to amplify cDNAs. The amplified DNAs are separated on a 2.0% agarose gel. The DNA fragments are isolated by QIAquick Gel Extraction Kit and are cloned into pCR4.0 TA vector (Invitrogen, Carlsbad, Calif., USA). The plasmid DNAs of the positive clones are isolated and sequenced. The sequenced data are used by blast and manual inspection to verify the fusion junctions (FIG. 8). The CPSF6|CACNA1E fusion transcript suggests that the in-frame CPSF6|CACNA1E fusion gene has eight exons of the CPSF6 nine exons and forty-eight exons of the CACNA1E forty-nine exons, which are much larger than both proteins.

In addition, we have verified two fusion transcripts, RRP8|RAB2A and FAM164A|RASA4PandPOLR2J4 in heart tissues from patients with heart diseases. As we have observed above, the fusion transcripts have been shown to have individuality. The validation of these lowly-expressed fusion transcripts have suggested that the many of the lowly-expressed fusion transcripts may play important roles in cancer initiation, developments, invasion, and metastasis.

To check whether these three fusion transcripts are expressed in other cancer cell lines, we have used identical conditions to perform individual RT-PCR amplification of cDNAs from these cancer cell lines described above without success. We have tested different experimental conditions without any success. Since we have such large numbers of lowly-expressed fusion transcripts, we need more efficient method to validate these fusion transcripts in varieties of tissues, cells and individuals.

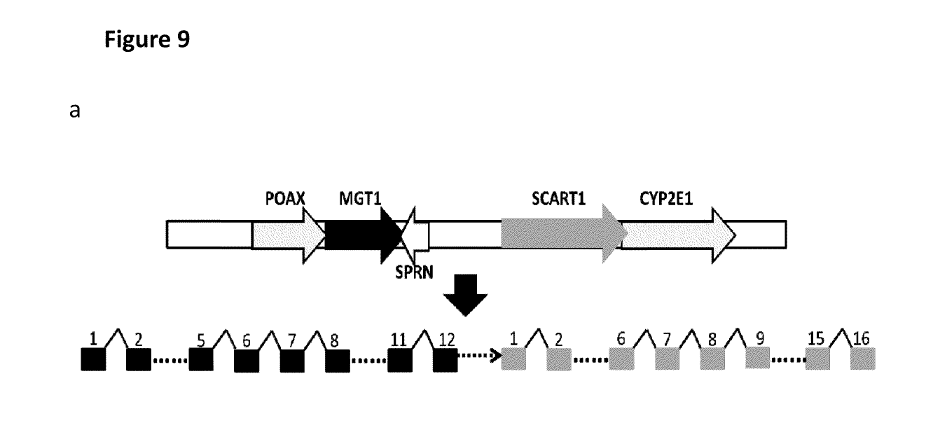

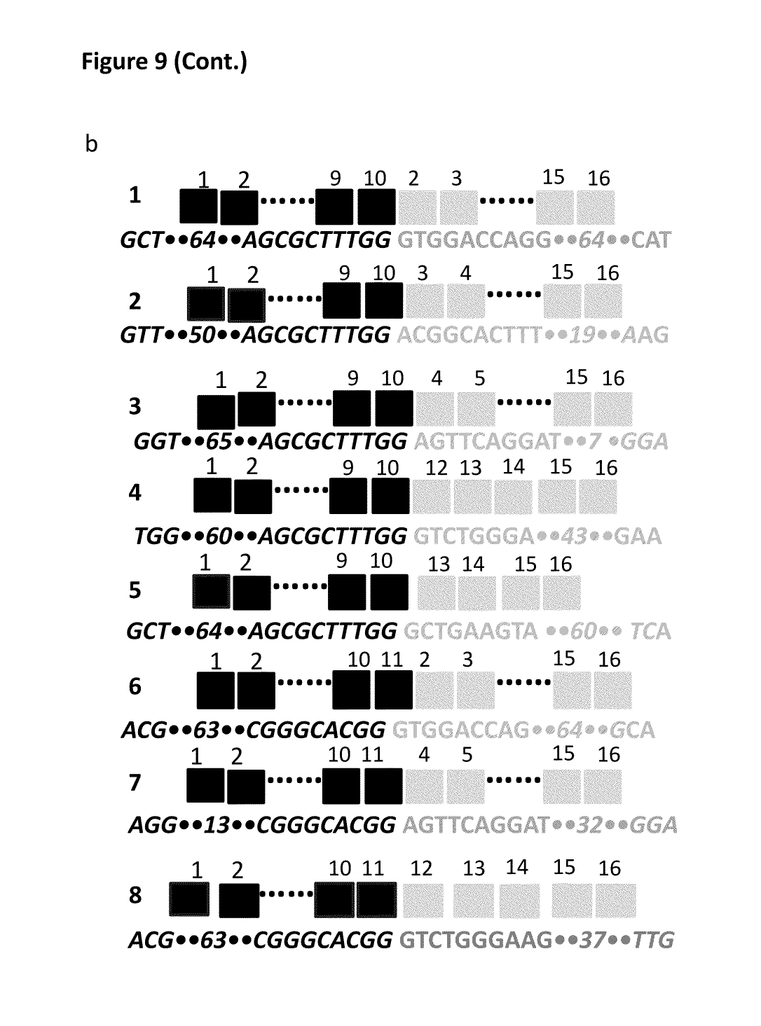

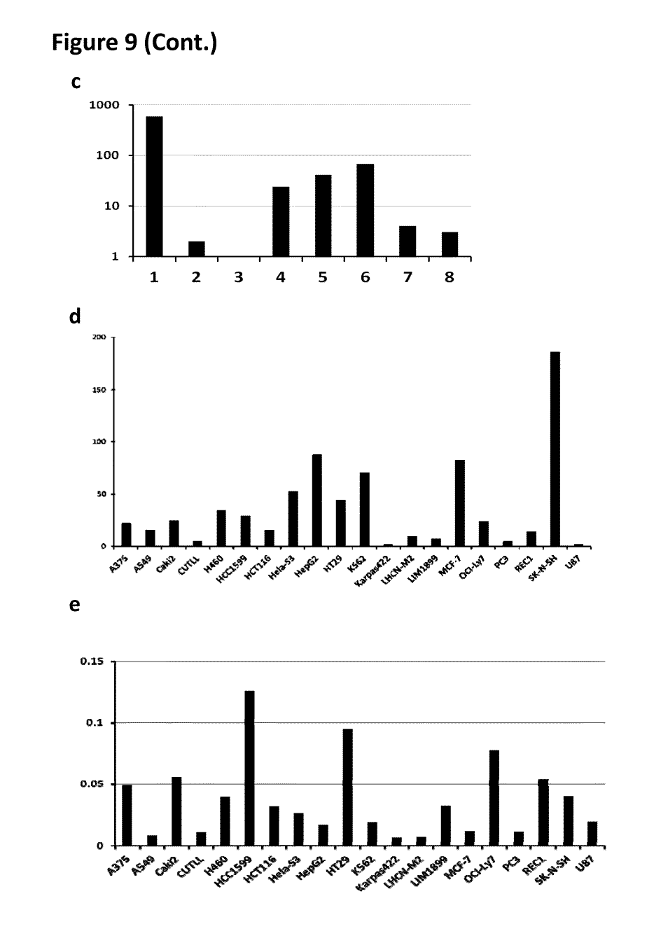

Table 3 shows that many top fusion transcripts are from read-though and recurrent in many cell lines. FIG. 9a shows MTG1 and SCART1 (LOC609217) on the chromosome 10, which encodes mitochondrial GTPase 1 homolog and a pseudogene of scavenger receptor protein family member, respectively. The read-though has resulted in fusion transcripts between MTG1 and SCART1 genes. Eight isoforms have been identified. Five fusion transcripts are 5′ alternatively-spliced at the MTG1 exon 10 while the remaining 3 fusion transcripts are alternatively-spliced at the MTG1 exon 11. These data have clearly shown that MTG1|SCART1 fusion gene is alternatively spliced and are able to generate in-frame hybrid proteins (FIG. 9b). These data have demonstrated that read-though fusion genes are similar to normal genes. MTG1|SCART1 isoform 1 has been the dominant isoform (FIG. 8c) and could generate a fusion protein containing majority of MTG1 and major part of SCART1 protein. FIG. 9d has shown that MTG1|SCART1 fusion transcripts have been detected in 29 out of 39 cancer cell lines. FIG. 9e shows that the expression levels among different types of cancer are significantly different and the ratios of different isoforms also differed significantly.

Read-through fusion transcripts are significantly different from the other four fusion transcripts. That is, two parental genes of fusion transcripts are close each other on the same chromosomes with the same orientations. Even though some read-though fusion transcripts may be caused by genetic alternations, majorities of read-though fusion transcripts may be due to failures of fail-safe transcriptional mechanisms (Porrua and Libri 2015). Many aberrant environmental and developmental factors often result in failures of transcriptional terminations and generate read-though fusion transcripts. More importantly, majority of fusion transcripts may be tissues-specifically expressed and have special functions. To verify whether expression of read-through fusion transcripts is tissues-specific, we have performed analysis of RNA-seq datasets of normal human tissues which include tissue samples from 95 human individuals representing 27 different tissues and primary cell lines (ENCODE 2015, SCILIFELAB 2015). Read-though fusion transcripts from different tissues have been used as negative controls to analyze cancer fusion transcripts.

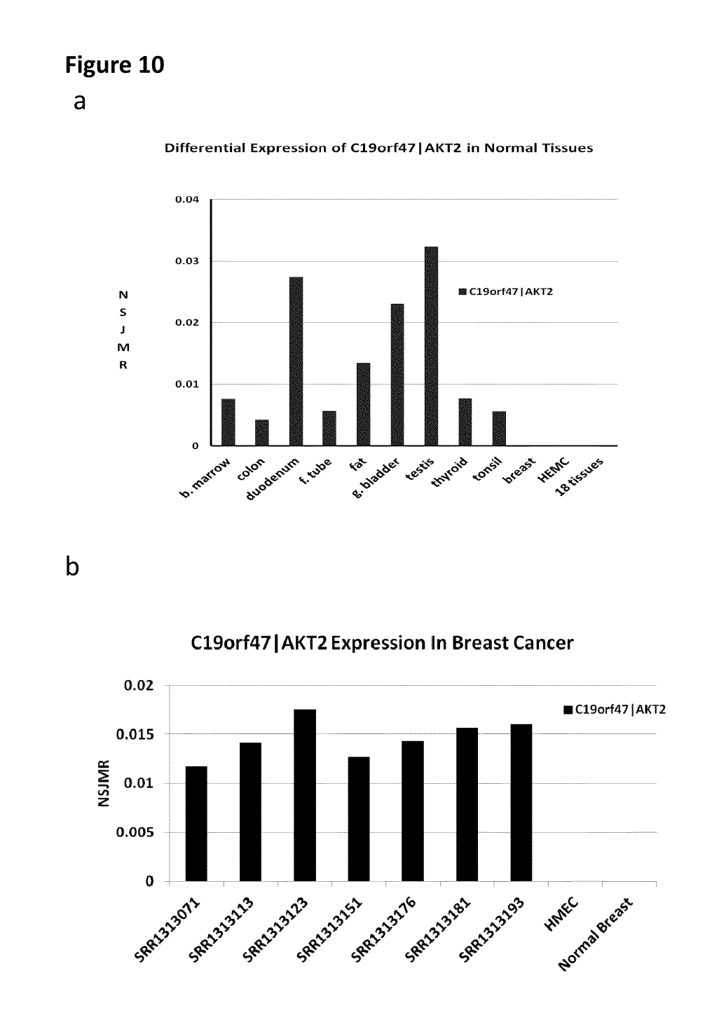

FIG. 10a shows an example demonstrating differential expression patterns of read-through fusion transcripts in normal tissues. The C19orf47 and AKT2 genes are located the chromosome 19 and are separated by 57 Kb. FIG. 10a has shown that the C19orf47|ATK2 fusion transcripts have been detected in bone marrow, colon, duodenum, Fallopian tube, fat gall bladder, testis, thyroid, tonsil but they are not found in other 18 other tissues as well as breast tissues and HMEC. In addition, FIG. 10a has also demonstrated that C19orf47|AKT2 fusion transcripts are expressed at significantly different levels among these nine tissues.

To demonstrate how to use read-though fusion transcripts as cancer biomarkers, we have performed analysis of breast cancer data from HudsonAlpha Institute for Biotechnology, AL, USA (designed as HIBCD) (Varley, et al. 2014), which have 168 breast cancer samples. FIG. 10b has shown that 7 (4%) breast cancer samples have been shown to express C19orf471ATK2 fusion transcripts out of the HIBCD 168 breast cancer samples.

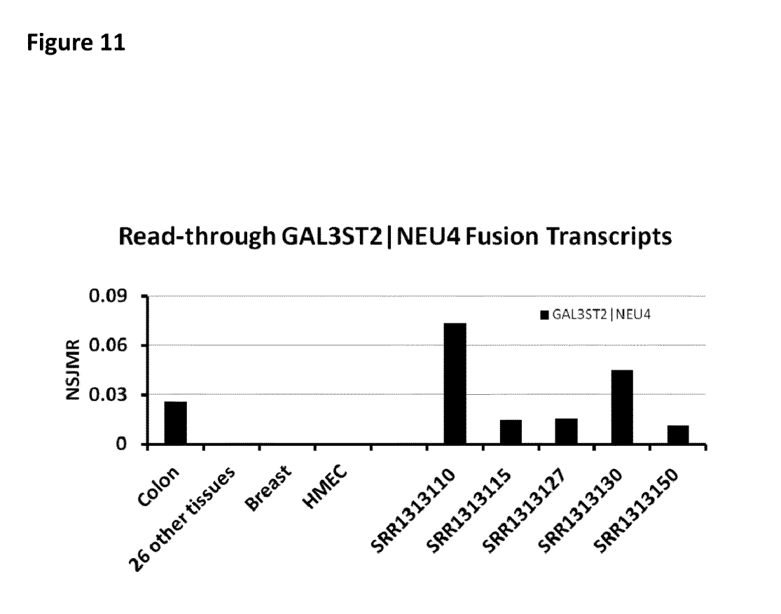

To further demonstrate how to use read-though fusion transcripts as cancer biomarkers, we have performed analysis of both HIBCD and South Korean breast cancer data (designed as SKBCP) (ERP010142 2015). Then we have performed comparative analyses of the fusion transcripts from normal human tissues and the HIBCD breast cancer samples. FIG. 11 has shown that GAL3ST2 gene, encoding galactose-3-O-sulfotransferase 3, and NEU4 gene, coding for N-Acetyl-Alpha-Neuraminidase 4, are located on the chromosome 11 and are separated by 17 Kb. GAL3ST2 gene has been implicated in tumor metastasis processes while NEU4 gene has been associated with NEU4 include galactosialidosis. The GAL3ST2|NEU4 fusion transcripts are expressed only in normal human colon and absent in 26 other human tissues, breast and human mammary epithelial cells (HMEC). As shown in FIG. 11, we have detected GAL3ST2|NEU4 fusion transcripts in 5 (3%) samples out of the 168 HIBCD breast cancer samples, two of which have much significantly higher expression levels than that in colon tissues. On the other hand, we have detected GAL3ST2|NEU4 fusion transcripts in only one (1.3%) sample out of 78 SKBCP breast cancer patients. These data have suggested that GAL3ST2|NEU4 fusion transcripts are far less frequent than what people have expected. This has demonstrated that read-though fusion transcripts can be used to test whether they are expressed in wrong tissues and wrong developmental stages.

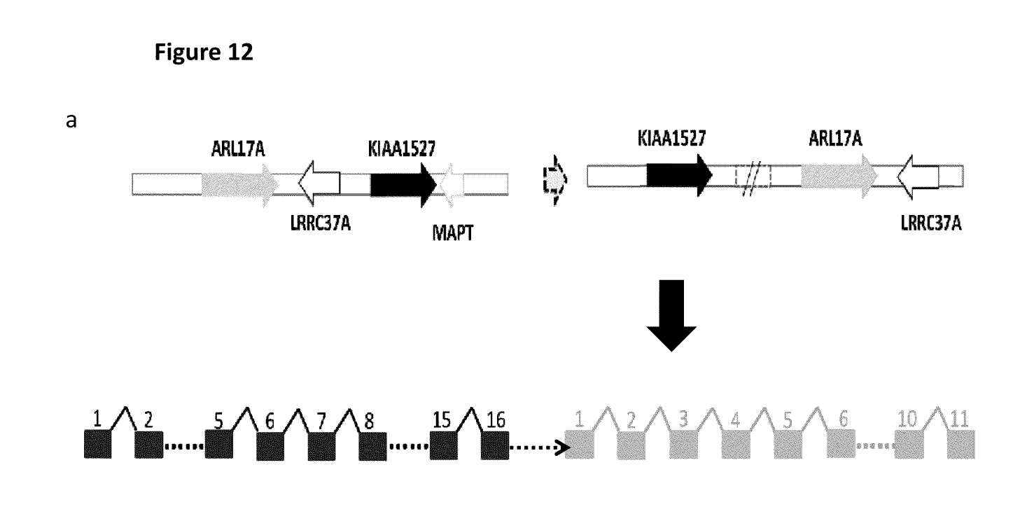

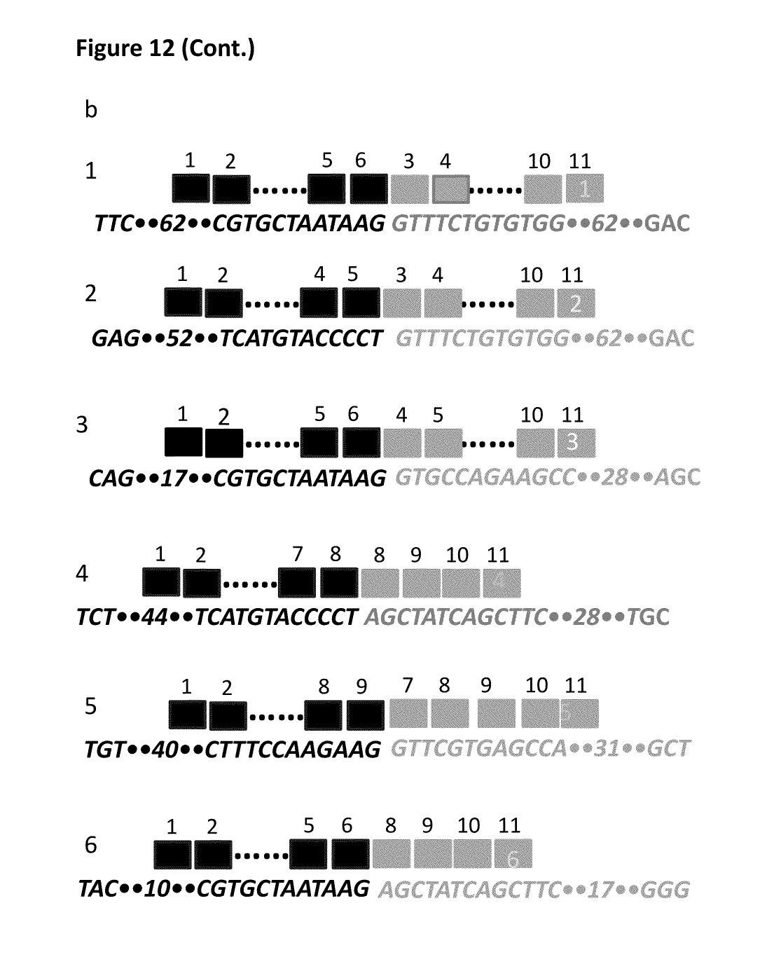

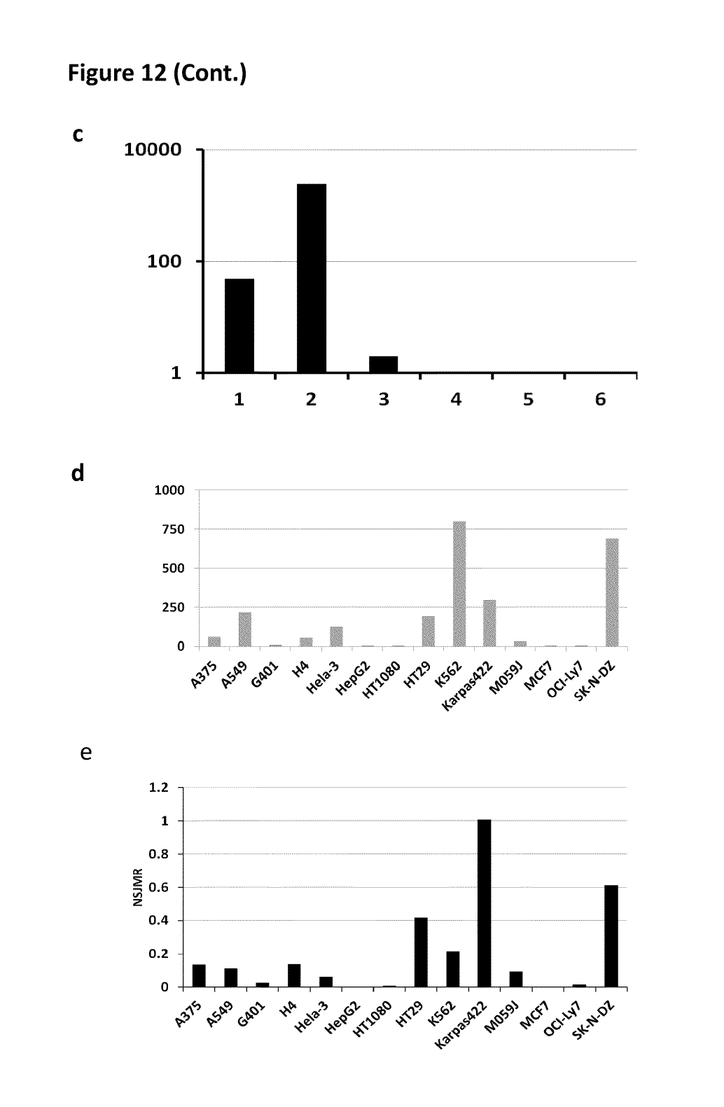

As shown in FIG. 4d, in addition to read-through, inversions have much more recurrent fusion transcripts than those of interchromosomal translocations, intrachromosomal translocations and deletions. So we have examined inversion fusion transcripts and identified large numbers of recurrent fusion transcripts as potential cancer detection biomarkers. Table 3 has shown that many high-expressed fusion transcripts come from inversions or duplications. One of the highly-expressed fusion transcripts is KANSL1 (KIAA1267)|ARL17A reported previously (Kinsella, et al. 2011), which is resulted from a chromosome 17 inversion (FIG. 12a). FIG. 12b has shown that the KANSL1|ARL17A fusion gene generates six fusion transcripts, which can produce potential KANSL1|ARL17A fusion proteins, five of which are novel fusion transcripts. FIG. 9c has shown that fusion transcript 2 is expressed at the highest levels among the six fusion transcripts. FIG. 12d has shown that the KANSL1|ARL17A fusion transcripts have been found in 14 out of 39 cancer cell lines and the largest and the second largest numbers of KANSL1|ARL17A fusion transcripts have been found in K562 and SK-N-DZ cancer cell lines. To rule out the size effects of RNA-seq datasets, we have normalized expression of the KANSL1|ARL17A fusion transcripts. FIG. 12e has shown that the highest expressed fusion transcripts have been found in Karapas-422 cancer cell line. A549, H4, HT29, A375, SK-N-SH, and K562 are among highly-expressed cancer cell lines (FIG. 12e). KANSL1 gene, located in 17q21.31, encodes KAT8 regulatory NSL complex subunit involved with histone acetylation and is associated with koolen de vries syndrome, formerly known as 17q21.31 microdeletion syndrome (Koolen, et al. 2006, de Jong, et al. 2012). Chromosomal band 17q21.31 contains common recurrent inversions in 20% population with European ancestry (Stefansson, et al. 2005). Based on the information of cancer cell lines, the majorities of the ECD39 cancer cell lines are Caucasian, which suggests their European ancestry. Our KANSL1|ARL17A fusion transcript data and genetic data (Koolen, et al. 2006, de Jong, et al. 2012) have suggested that KANSL1|ARL17A fusion transcripts are associated with recurrent inversions of the chromosomal band 17q21.31.

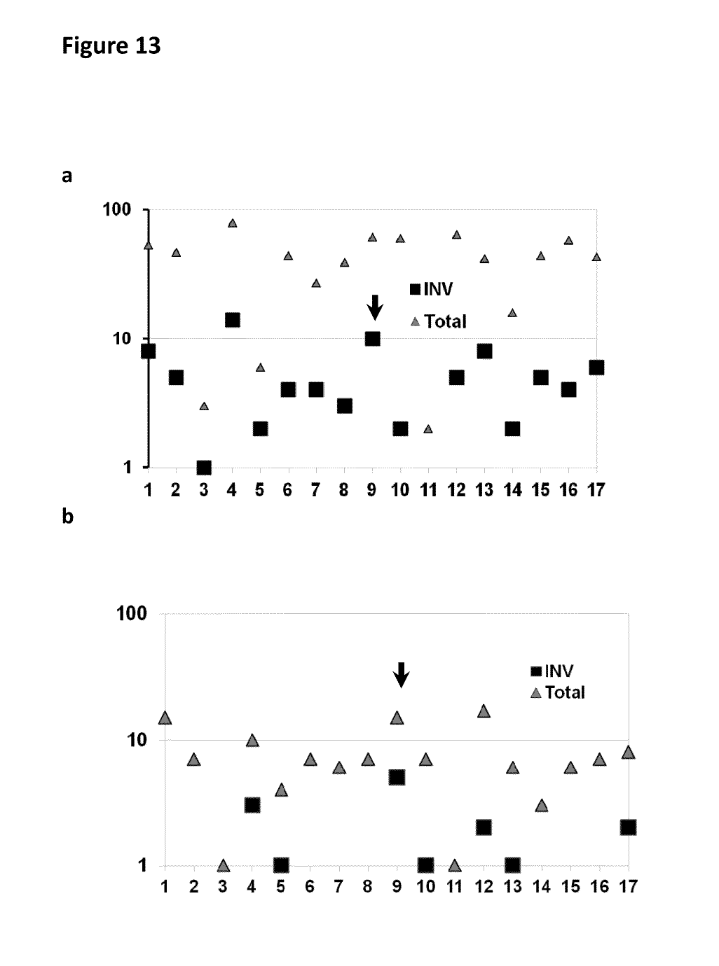

To explore whether the fusion transcripts can be used to investigate relationships between human evolutionary genetics and fusion transcripts, we have plotted the total fusion transcripts and inversion fusion transcripts along the human chromosome 17. FIG. 13a shows the relationships between the total fusion transcripts and inversion fusion transcripts and chromosome positions, each of which represents 5M bp. FIG. 13a shows that there is peaks of both total and inversion fusion transcripts between 41 Mb and 49 Mb. When we have plotted total fusion transcripts identified in ≧2 cancer cell lines and inversion fusion transcripts detected in ≧2 cancer cell lines along the human chromosome 17, FIG. 13b has shown patterns similar to those in FIG. 13a and locations of KANSL1|ARL17A fusion transcripts are indicated by arrows. These suggest that the region from 41 Mb to 49 Mb of the chromosome 17q21.31 band is associated with numbers of other recurrent fusion transcripts. In addition, we have found that three additional peaks may be associated with human genetic variations on the chromosome 17.

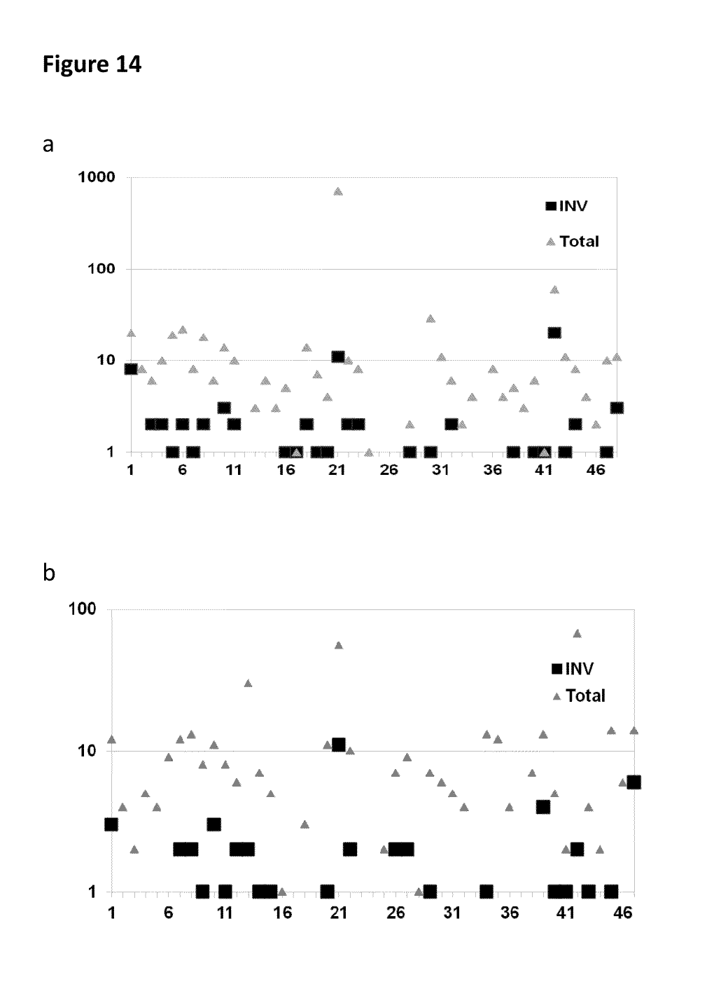

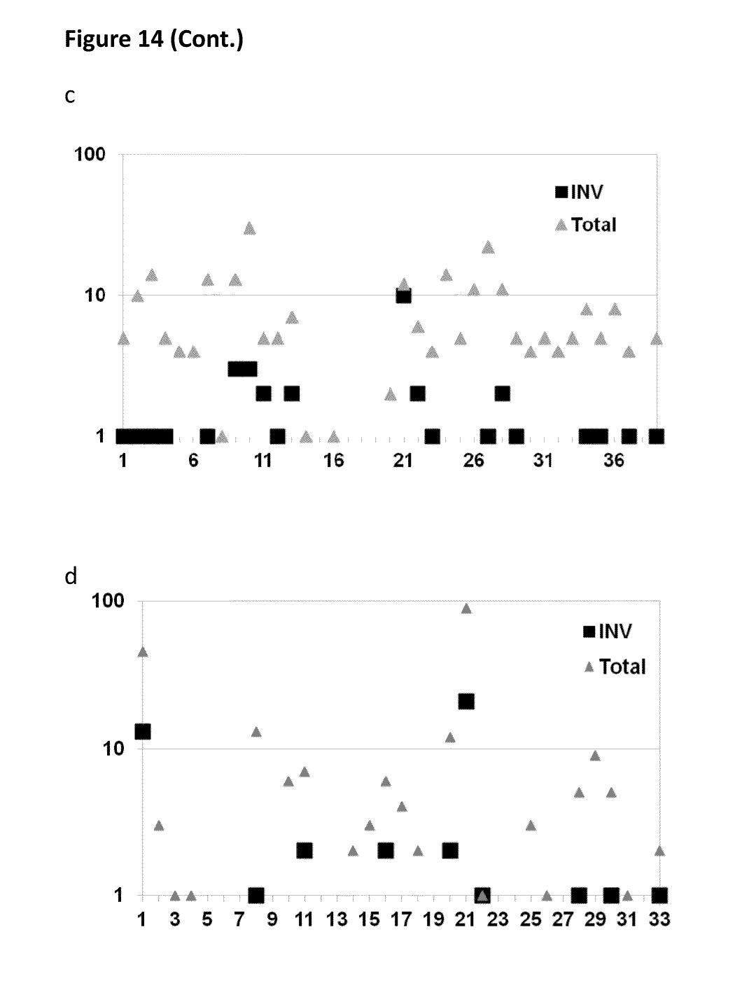

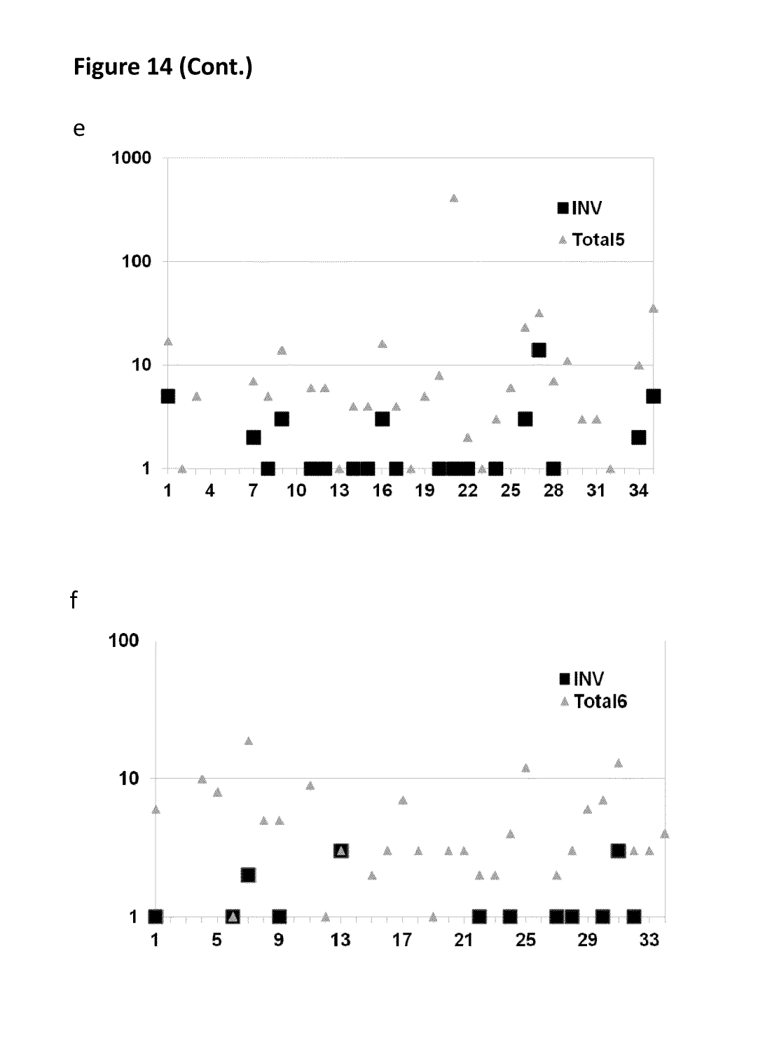

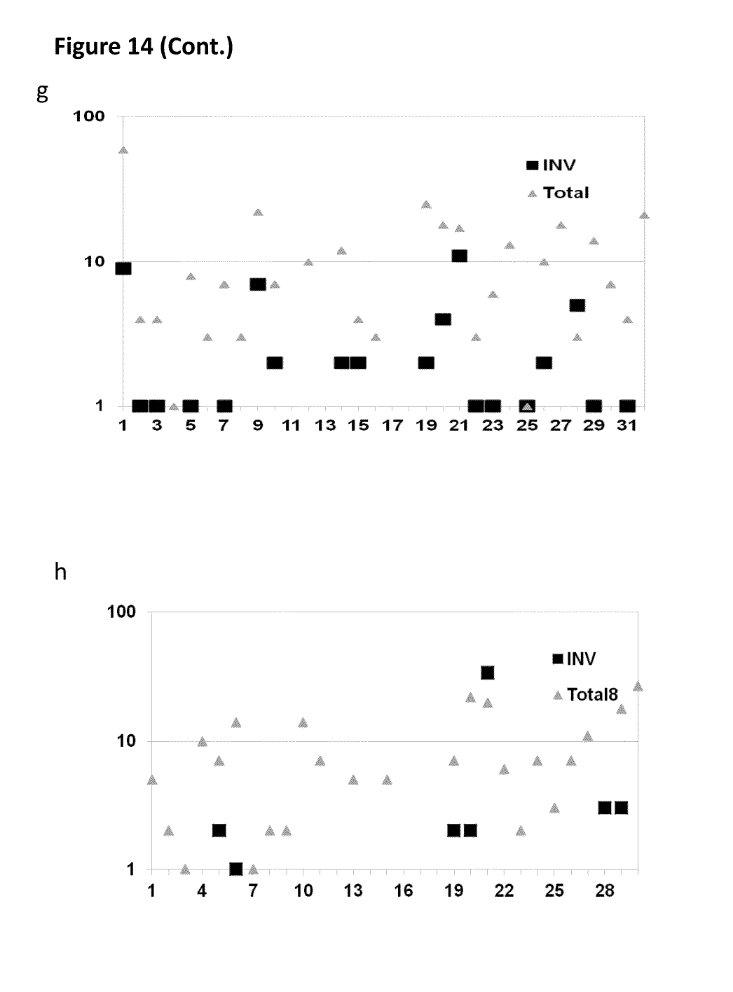

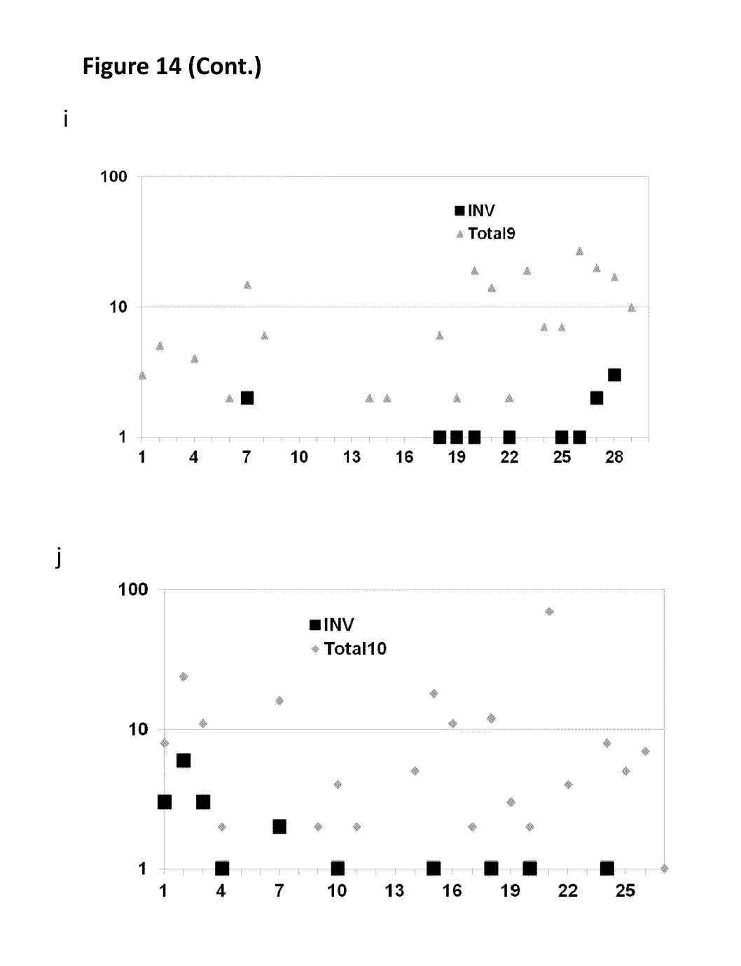

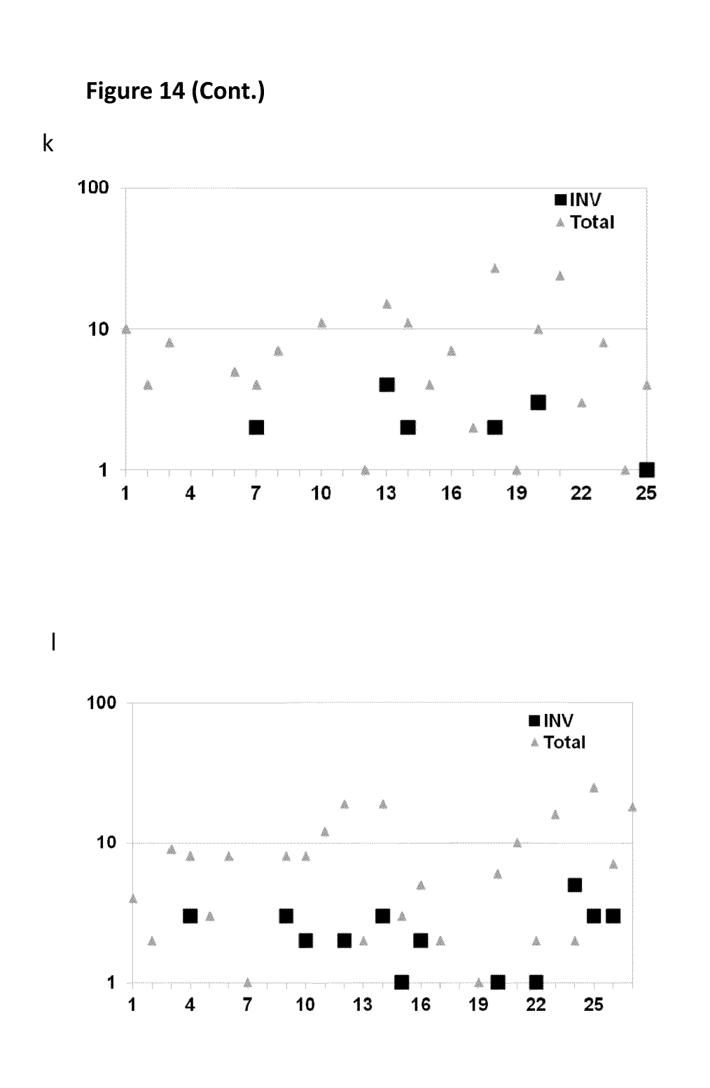

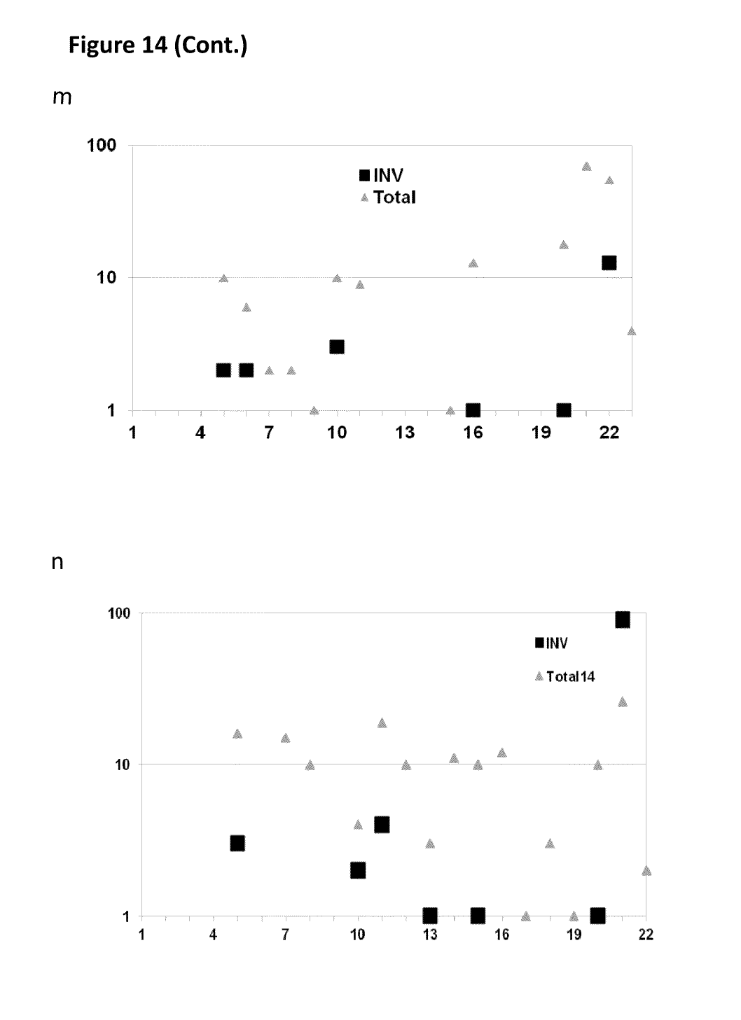

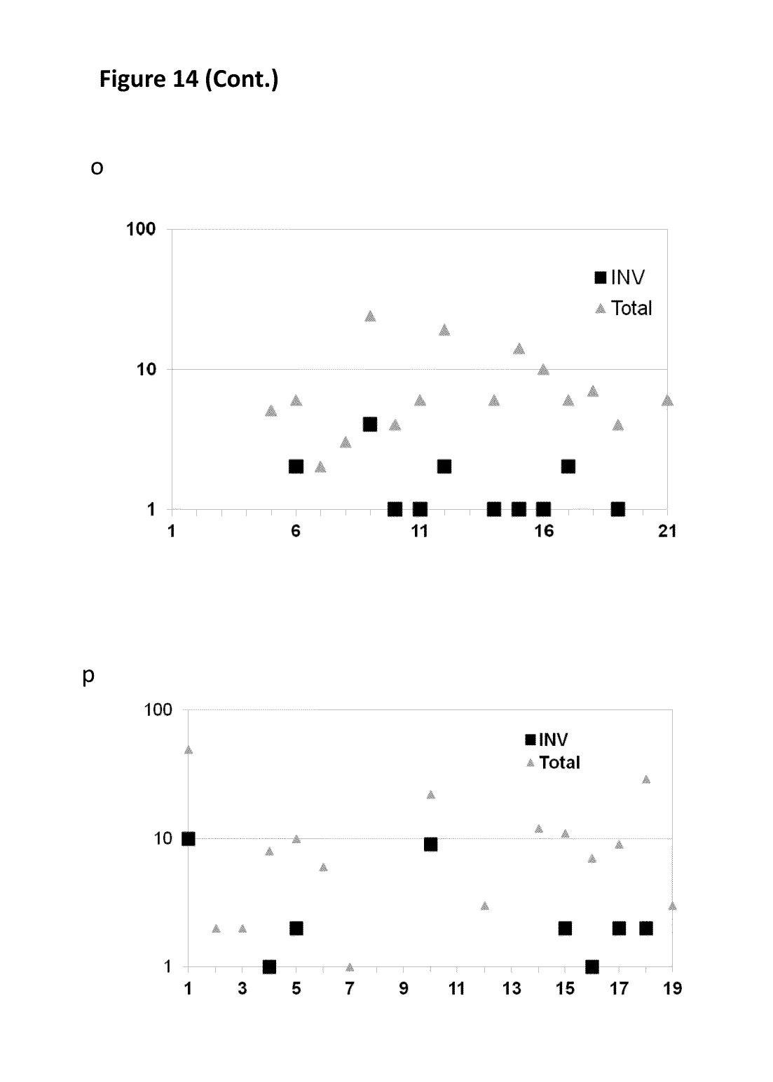

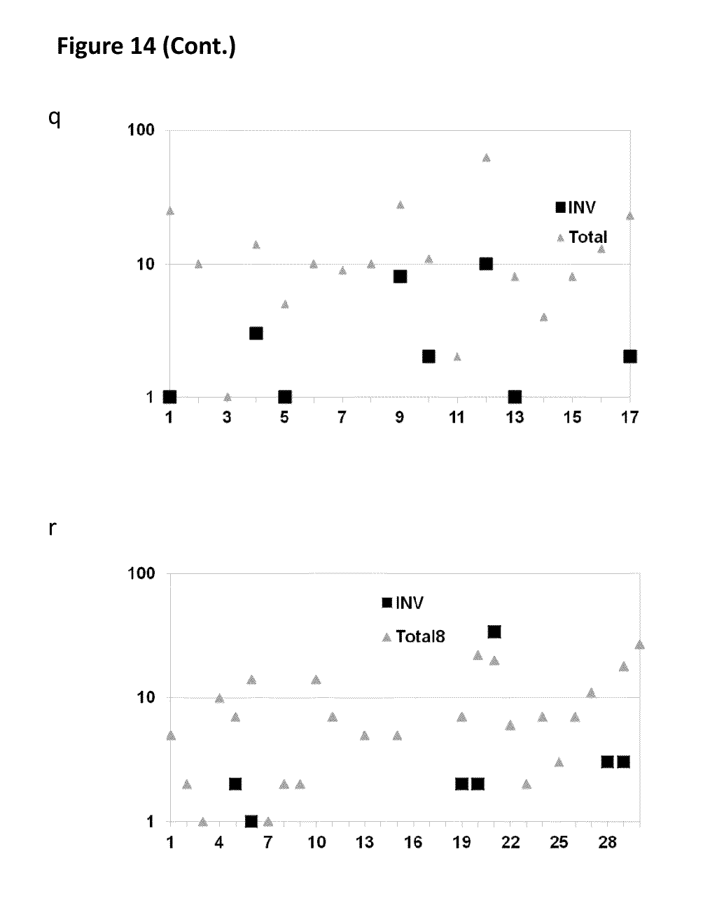

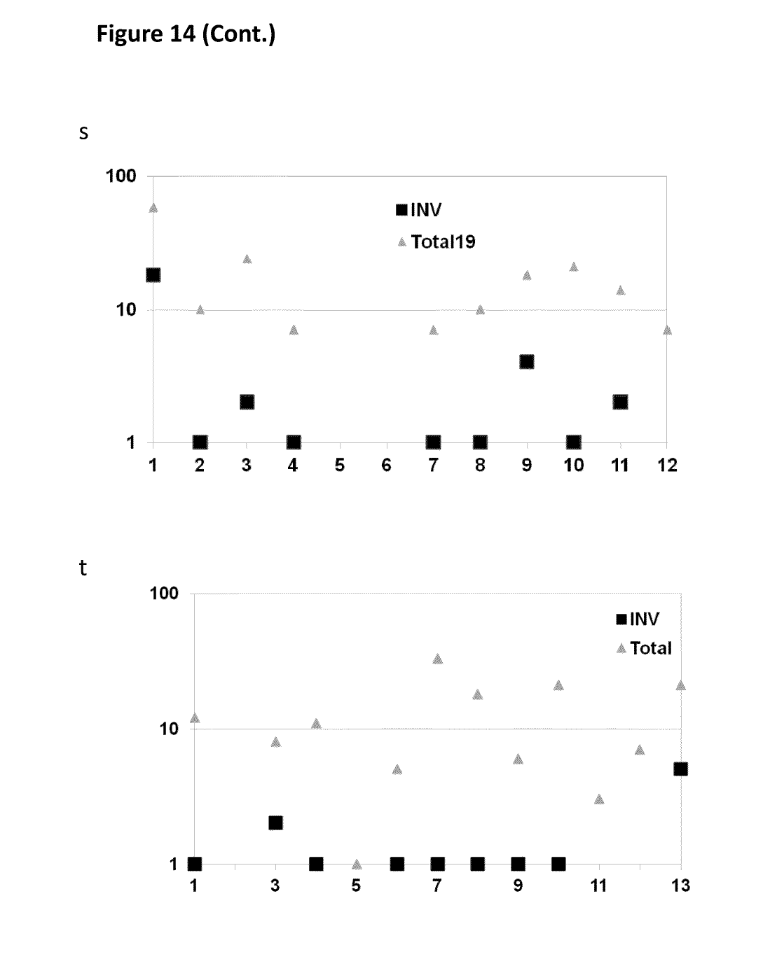

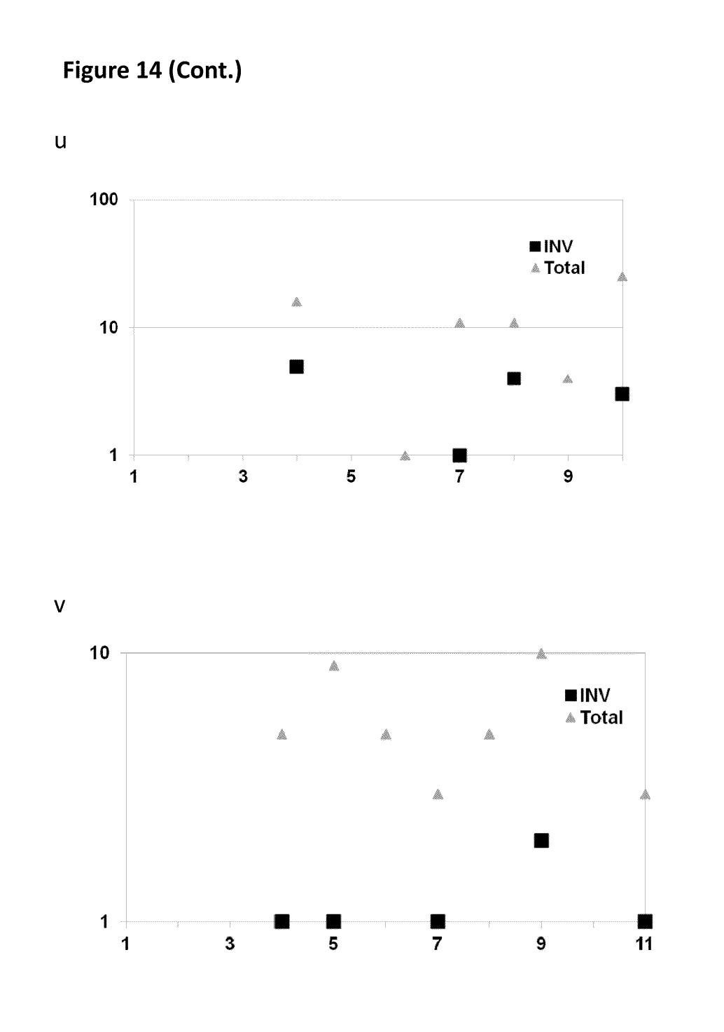

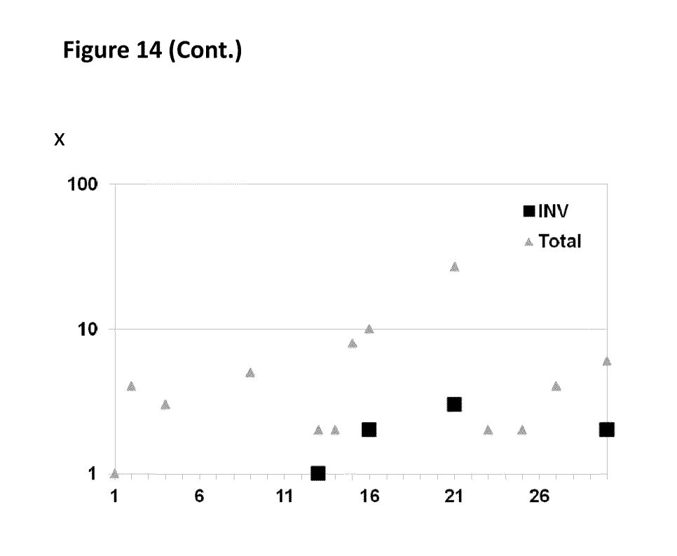

As we discussed above, we can use the hit maps of fusion transcripts to discover and locate recurrent chromosomal regions associated with cancers. We have plotted the hit maps of total fusion transcripts and inversion fusion transcripts. FIG. 14 shows the genome-wide hit maps of the total fusion transcripts detected in ≧2 cancer cell lines and inversion fusion transcripts detected in ≧2 cancer cell lines. The peaks in each hit map represent variable regions and may be associated with cancer.

FIG. 13a and FIG. 13b have shown that chromosomal band 17q21.31 contains multiple fusion transcripts. Table 6 shows 18 putative fusion genes from 41 Mb to 49 Mb of the chromosome 17q21.31 region, which are pointed by arrows and are supported by 34 fusion transcripts. Clustering large numbers of fusion transcripts suggests that certain genetic variations make these regions unstable and often result in genetic alternations, which generate fusion transcripts.

TABLE 6
List of fusion transcripts detected between 42 Mb to 48 Mb of
the chromosome 17.

| 5′ Gene | 5′ Positions (Mb) | 3′ Gene | 3′ Positions (Mb) |
|---|---|---|---|
| LRRC37A4 | 43.6 | NSF | 44.7 |
| LRRC37A4 | 43.7 | NMT1 | 43.2 |
| LRRC37A4 | 43.7 | KIAA1267 | 44.2 |
| LRRC37A4 | 43.7 | LRRC37A3 | 62.9 |
| LRRC37A4 | 43.7 | ARSG_{—} | 66.3 |
| C17orf69 | 43.7 | ARHGAP27 | 43.5 |
| KIAA1267 | 44.1 | ARL17A | 44.6 |
| ARL17A | 44.6 | KIAA1267 | 44.2 |
| NSF | 44.8 | LRRC37A3 | 62.9 |
| MRPL45P2 | 45.5 | NPEPPS | 45.6 |
| NPEPPS | 45.7 | ITGB3_{—} | 45.5 |
| MRPL10 | 45.9 | KIAA0100_{—} | 27 |
| HOXB6 | 46.7 | BAIAP2 | 79 |
| ATP5G1 | 47 | UBE2Z | 47 |
| GIP | 47 | SNF8 | 47 |
| SPOP | 47.7 | NME1-NME2 | 49.2 |

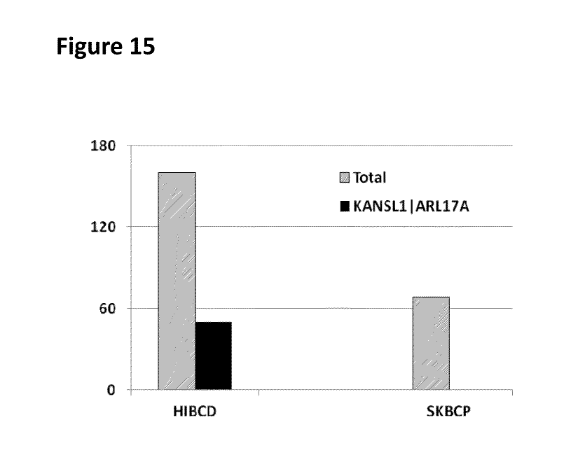

It has been reported that the H2 lineage is rare in Africans, almost absent in East Asians, but found in 20% population with European ancestry (Stefansson, et al. 2005). To further confirm the inversion KANSL1|ARL17A fusion transcript is a cancer-biomarker associated with European genetic backgrounds, we have performed analysis of HIBCD and SKBCP breast cancer data (Varley, et al. 2014, ERP010142 2015). The HIBCD contains 168 breast cancer cell lines and primary breast cancer tissues samples (Varley, et al.). The SKBCP has samples from 22 HRM (high-risk for distant metastasis) and 56 LRM (low-risk for distant metastasis) breast cancer patients (ERP010142 2015). We have performed comparative analyses of HIBCD and SKBCP samples. FIG. 15 has shown that HIBCD has 50 samples that express KANSL1|ARL17A fusion transcripts while the SKBCP has none of the KANSL1|ARL17A samples. The difference between HIBCD and SKBCP has been shown by χ²-test to be statistically significant (p≧0.001). SKBCP has 100 bp RNA-seq reads and has total 1.6×10¹² base counts while HIBCD has 50 bp RNA-seq reads in length and has total 1.2×10¹² base counts. Therefore, the qualities of the SKBCP dataset are better than those of the HIBCD. These data have ruled out that the KANSL1|ARL17A fusion transcripts are caused by experimental errors and random chances. The absence of the SKBCP KANSL1|ARL17A samples not only has further confirmed that any fusion transcript identified by our splicingcodes method are not generated by random chance or experimental errors, but also have shown that KANSL1|ARL17A fusion transcripts are associated with breast cancer patients of European ancestry.

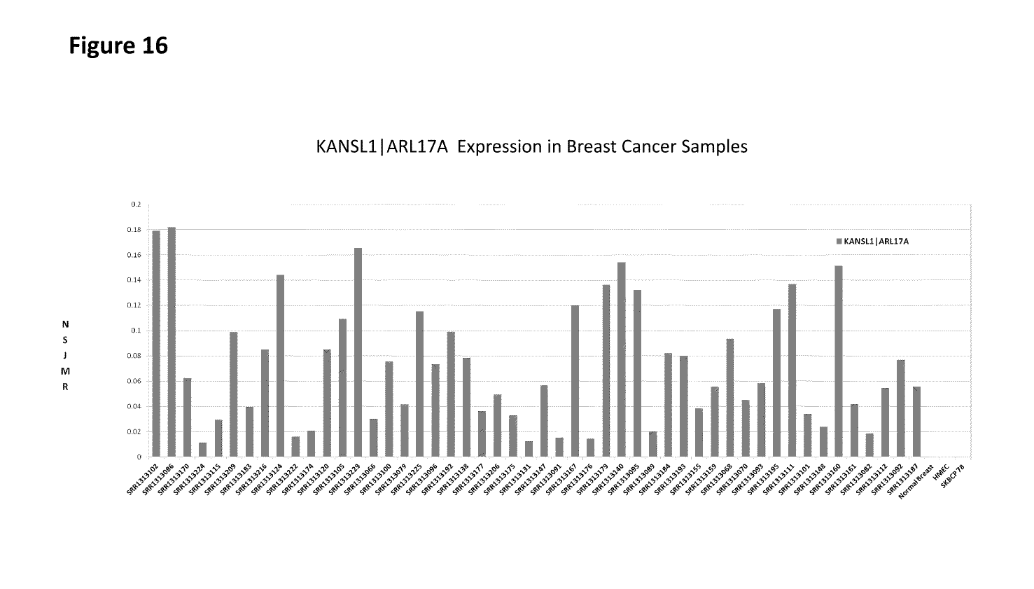

Since the KANSL1|ARL17A fusion proteins are involved with histone acetylation and may affect the chromosomal stabilities, it is highly unlikely that they directly cause cancer in a short time and may be earlier cancer biomarkers (de Jong, et al. 2012). However, their expression will have tremendous affects on the cancer initiation, developments, invasion, and metastasis. In order to understand their expression, we have analyzed expression levels in the HIBCD 50 cancer samples. FIG. 16 has shown that the KANSL1|ARL17A expression levels of HIBCD 50 samples are significantly different and range from 0.0113 to 0.18 NSJMR. The lowest and highest expression levels differ by 16 folds. FIG. 16 has also shown that the KANSL1|ARL17A fusion transcripts are not detected in the normal breast tissues and HMEC even though their RNA-seq datasets are much larger than individual ones of HIBCD samples.

Even though we don't know exact compositions of race backgrounds, we can reasonably predict that majority of the HIBCD′ samples have European ancestry due to their USA origins. On the other hand, all most SKBCP patients have Asian ancestry. Since the KANSL1|ARL17A fusion transcripts have been detected in 35.8% of the ECD39's 39 cancer cell lines (FIGS. 9c and 9d) and 30% of the HIBCD's 168 samples, we can conclude that the KANSL1|ARL17A fusion transcripts and other fusion transcripts between 41 Mb and 49 Mb of chromosomal band 17q21.31 band (Table 6) can be used to detect any types of cancer and are cancer biomarkers of patients with European ancestry. Since these fusion transcripts are the consequences of “traditional” human evolutionary studies (Stefansson, et al. 2005, Rao, et al. 2010), further understanding how certain genetic types will result in fusion genes and are associated with cancer initiation, developments, uncontrolled growth, invasion, and metastasis will greatly help us to detect and prevent cancers in these subgroups of populations.

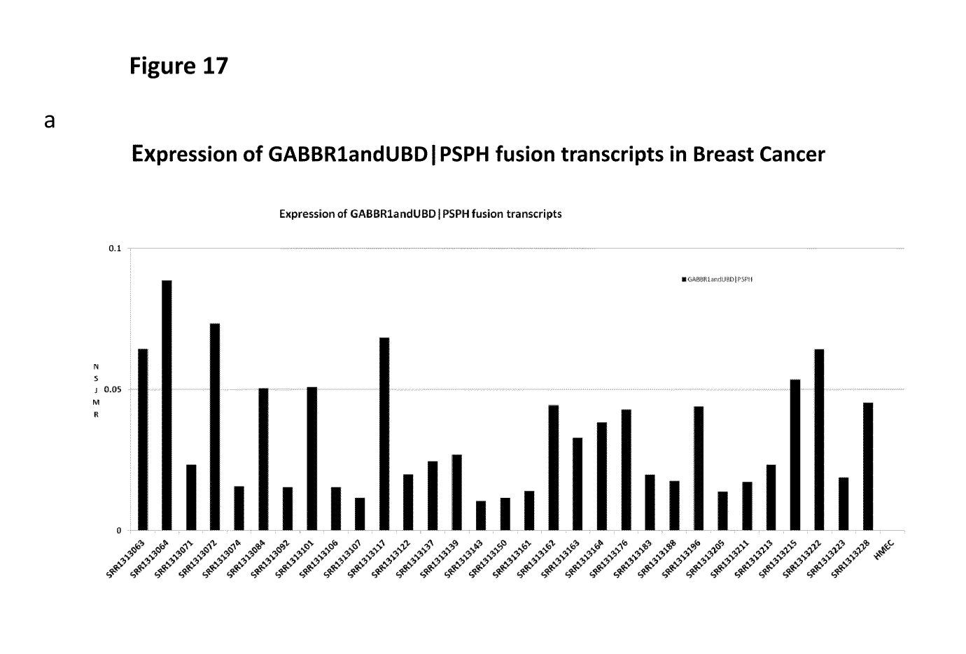

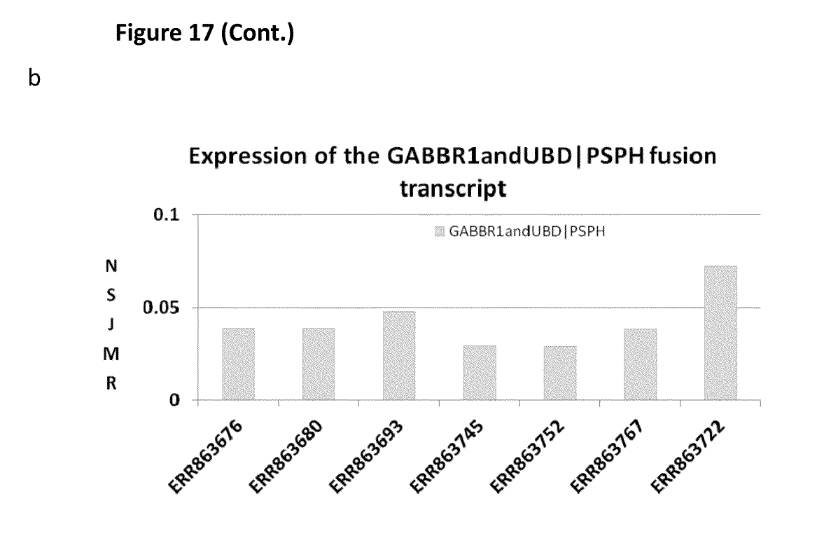

Like the inversion fusion transcripts, the recurrent fusion transcripts have been observed in the interchromosomal fusion transcripts. One example is the GABBR1andUBD|PSPH fusion transcripts. The GABBR1andUBD transcription unit is located on chromosome 6 while PSPH gene is on chromosome 7. The GABBR1andUBD fusion transcripts are generally expressed at very low levels in some lymphoblastoid cell lines and have one or two copies of GABBR1andUBD|PSPH fusion transcripts. However, we have found that GABBR1andUBD|PSPH fusion transcripts are highly expressed in stem cell lines while they are expressed at various levels in many cancer lines. These data have suggested that GABBR1andUBD|PSPH fusion transcripts may play roles in promoting cell differentiation and growth. Therefore, we have then performed analysis of the 168 HIBCD breast cancer samples and 78 SKBCP breast cancer samples. FIG. 17a has shown that the GABBR1andUBD|PSPH fusion transcripts have been detected in 31 breast cancer samples, which represents 18.4% of HIBCD breast cancer samples. Unlike the KANSL1|ARL17A fusion transcripts, FIG. 17b has shown seven samples have been shown to have GABBR1andUBD|PSPH fusion transcripts, which represent about 10% of the SKBCP samples and are less than that found in HIBCD. The GABBR1andUBD|PSPH fusion transcripts have not been detected in normal human breast tissues and different HMEC cells. The GABBR1andUBD|PSPH expression levels, which are estimated by numbers of splice junctions per million reads (NSJMR), vary significantly among different HIBCD samples and range from 1.15×10⁻² to 8.9×10⁻², which differ by 7.7 folds. In the future, we need to investigate whether expression levels of GABBR1andUBD|PSPH fusion transcripts are associated with cancer prognosis.

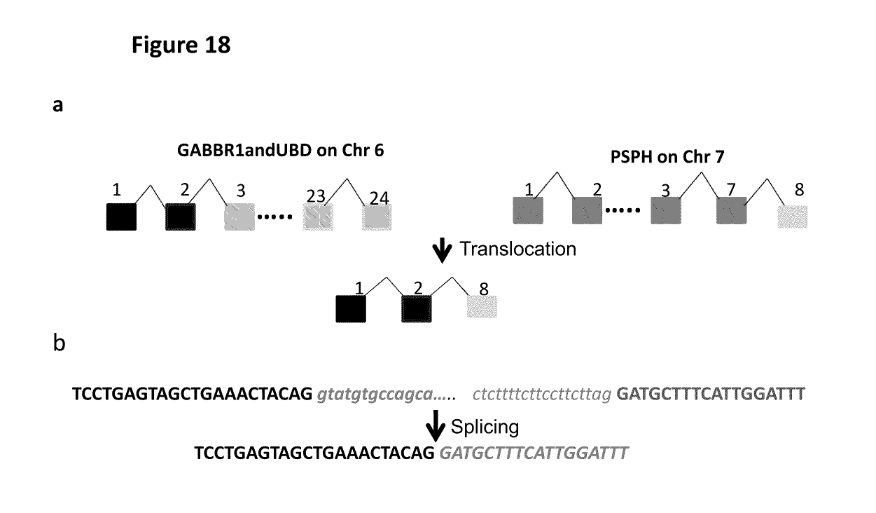

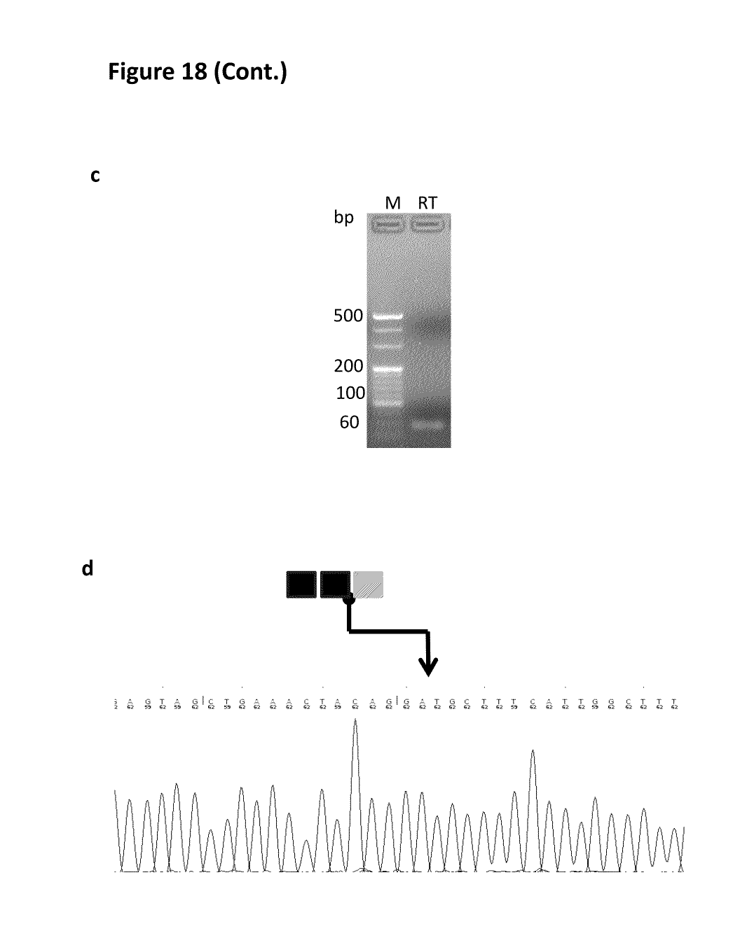

As shown in FIG. 17, the GABBR1andUBD|PSPH fusion transcripts have been detected in many breast cancer samples. As shown in Table 4, GABBR1andUBD-PSPH fusion transcripts are expressed at very low levels. We have isolated total RNAs from BT-474 cancer cell line as described above. To verify GABBR1andUBD-PSPH fusion transcripts, we have designed primers based on the fusion transcripts as shown in Table 5. We have used these primers to amplify cDNAs to detect GABBR1andUBD|PSPH fusion transcripts. The amplified GABBR1andUBD|PSPH cDNA fragments are separated on 2.0% agarose gels. The resulted PCR fragments have been isolated and purified by Qiagen Gel Extraction Kit. The purified cDNA fragments are then cloned into pCR4-TOPO clone vector. FIG. 18a has shown that interchromosomal translocations may have brought GABBR1andUBD gene on the chromosome 6 and PSPH gene of the chromosome 7 together and form a Head-Tail-to-Head structure. The putative GABBR1andUBD|PSPH fusion gene is spliced to remove introns to generate a transcript containing the first two exons of the GABBR1andUBD gene and the last exon of the PSPH gene. The amplified GABBR1andUBD|PSPH cDNA fragments are separated on 2.0% agarose gels. The resulted PCR fragment has been cloned into pCR4-TOPO clone vector and verified by DNA sequencing as shown in FIGS. 18b and 18c. The junction sequences of fusion transcripts are verified by blast and visual inspections (FIG. 18c). Then, we have tested whether the GABBR1andUBD|PSPH fusion transcripts are presents in normal MCF10A cell line and the cancer cell lines described above. It has been negative in MCF10A and cancer cell lines. However, further experiments have shown that the GABBR1andUBD|PSPH fusion transcripts are expressed in some lymphoblastoid cell lines. However, we need to develop much faster and more accurate methods to validate these fusion transcripts. Since the fusion transcripts are shown by blast to have homologous sequences from pseudogenes or other duplications, it has not affected using them as fusion transcript markers. However, if we want to investigate the functions of the fusion transcripts, we have to use RACE PCR to get full-length sequences.

As shown in Table 4, we have validated the LRRC37A3|VNN2 fusion transcripts in BT-474.

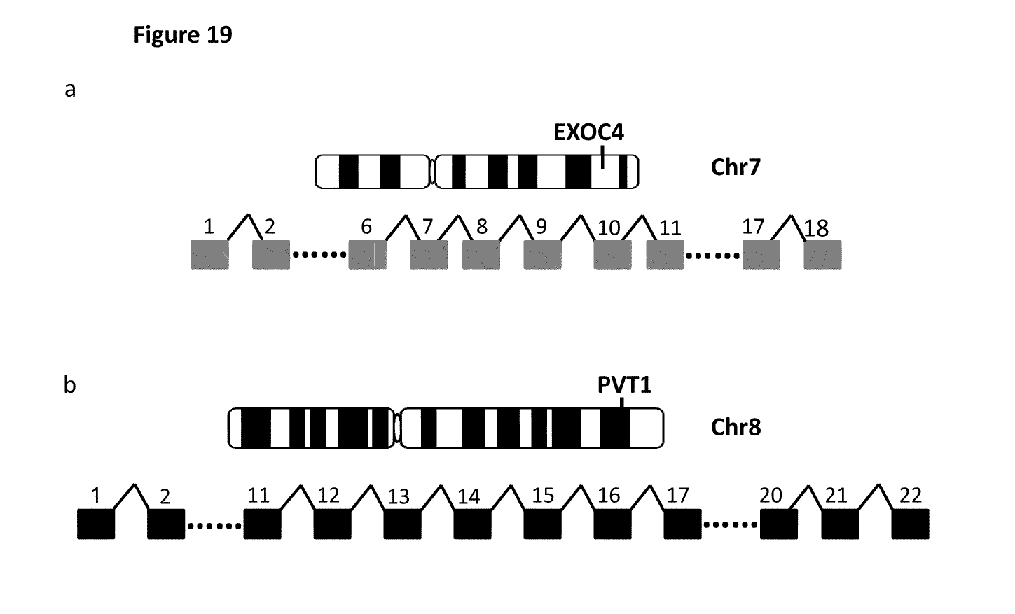

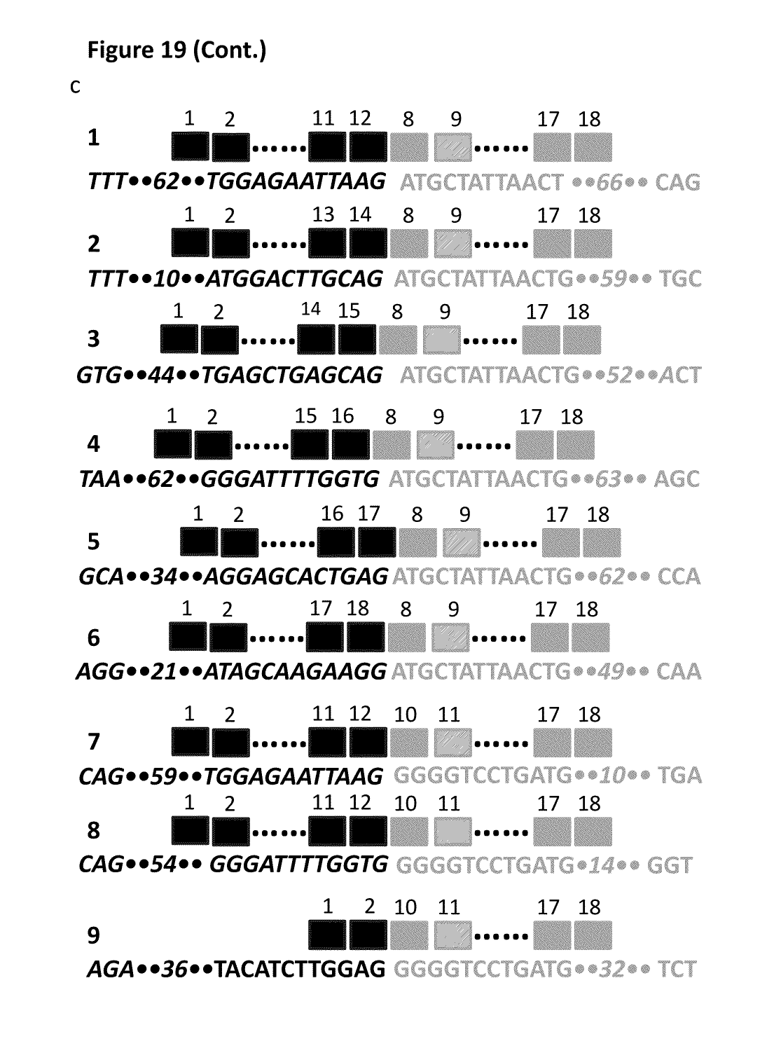

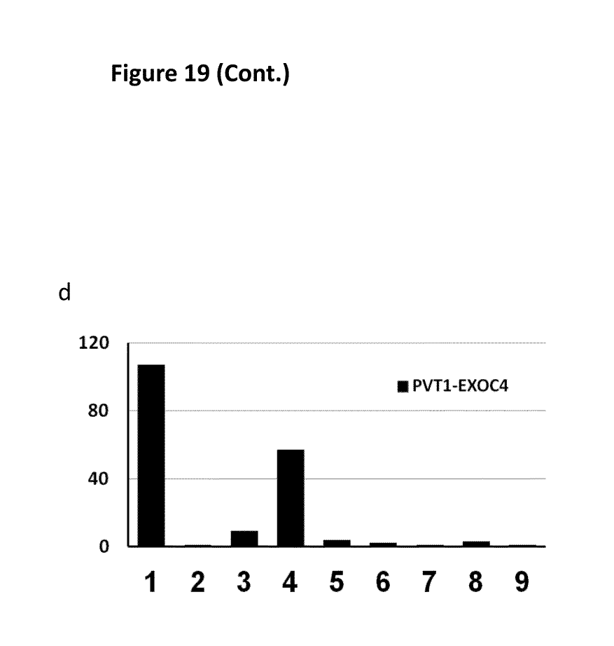

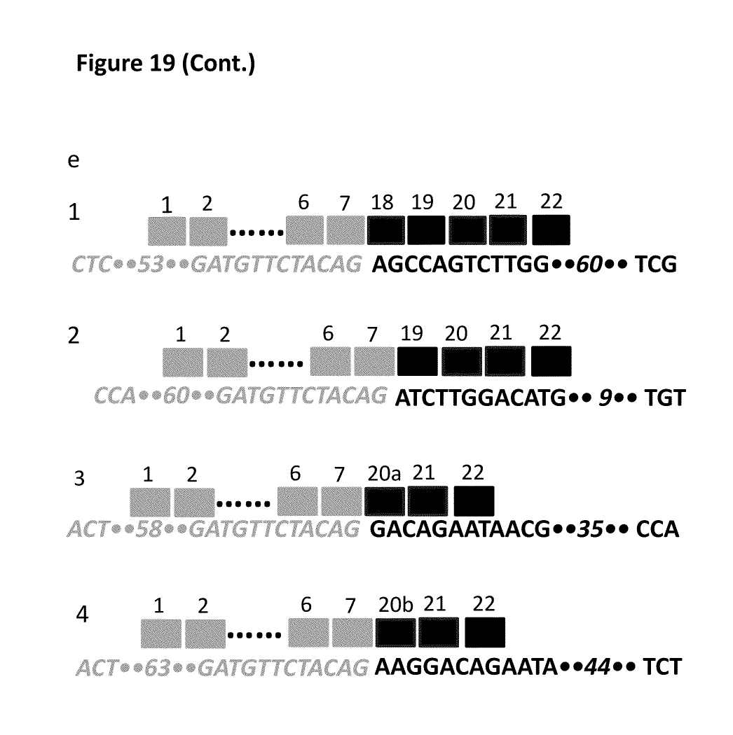

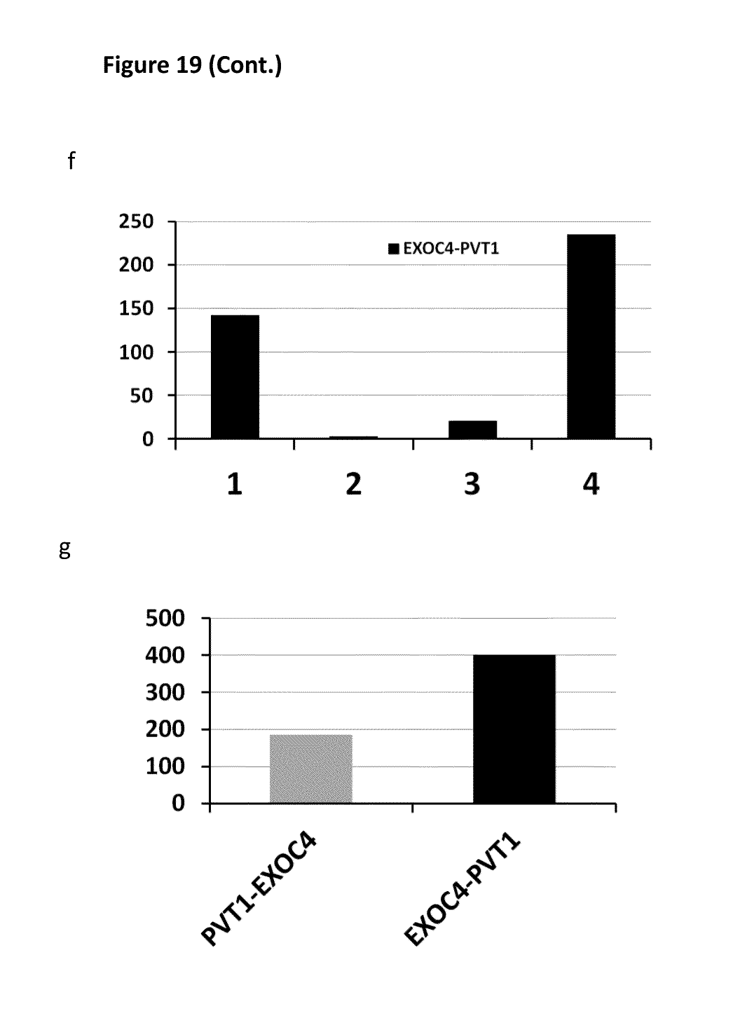

Table 3 has shown that the most complex fusion events have been observed in neuroblastoma SK-N-SH cells and are ones between PVT1 oncogene and EXOC4 gene. FIG. 19a shows that EXOC4 gene is located on the chromosome 7 and codes for a component of the exocyst complex involved in the docking of exocytic vesicles with fusion sites on the plasma membrane. FIG. 19b shows that PVT1 oncogene is on the chromosome 8 and codes for oncogenic non-coding RNA. FIG. 19c has shown that we have identified 9 PVT1|EXOC4 isoforms in SK-N-SH neuroblastoma cancer cell line. FIG. 19c shows that five PVT1|EXOC4 isoforms are alternatively-spliced at the 8^{th} exon of EXOC4 gene and three isoforms are alternatively-spliced at the 11^{th} exon of EXOC4 gene. FIG. 19d shows that PVT1|EXOC4 isoform 4 is the highest isoform and the second highest isoform is the PVT1|EXOC4 isoform 4. The remaining PVT1|EXOC4 isoforms are expressed at very low levels. Surprisingly, we have also identified EXOC4|PVT1 fusion transcripts. FIG. 19e shows that we have identified four EXOC4|PVT1 fusion transcripts, all of which are alternatively spliced at the 7^{th} exon of the EXOC4 gene. FIG. 19f shows that EXOC4|PVT1 isoform 4 is the highest isoform and the second highest one is the EXOC4|PVT1 isoform 1 (FIG. 19e). FIG. 19e shows that EXOC4|PVT1 isoform 3 and 4 differ by only three nucleotides but their expression levels differed by 11.75 folds (FIG. 190. FIGS. 19c and 19e have shown that PVT1 sequences are highly variable in all PVT1|EXOC4 and EXOC4|PVT1 fusion isoforms. These suggest that all PVT1|EXOC4 and EXOC4|PVT1 fusion isoforms may be regulated differentially. FIG. 19g shows that EXOC4-PVT1 gene (black bar) expression estimated by total sequence copies of supporting sequence reads is two folds of the PVT1-EXOC4 one (gray bar).

In addition, in gastric cancer cell SUN16, the top two fusion transcripts are from non-coding RNA PVT1 oncogene and SLC1A2, coding for glial high affinity glutamate transporter member 2 (Table 3). These complex fusion transcripts not only provide their fusion complex gene structures, but also suggest that non-coding RNA oncogene PVT1 may play important role in cancer development.

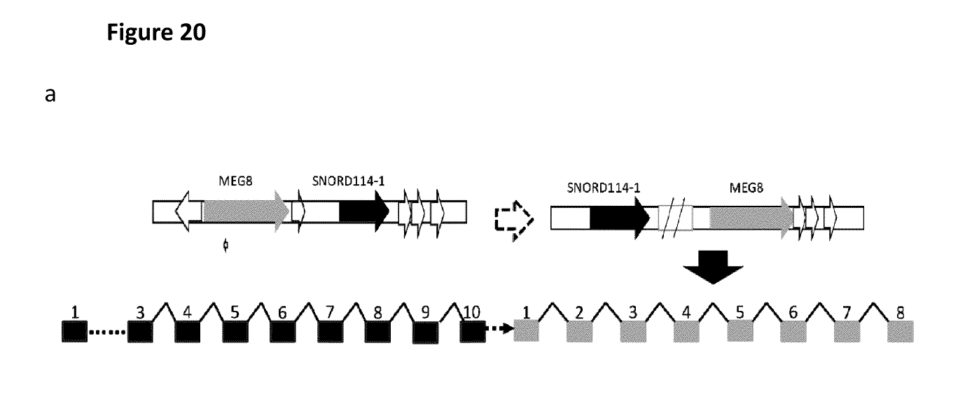

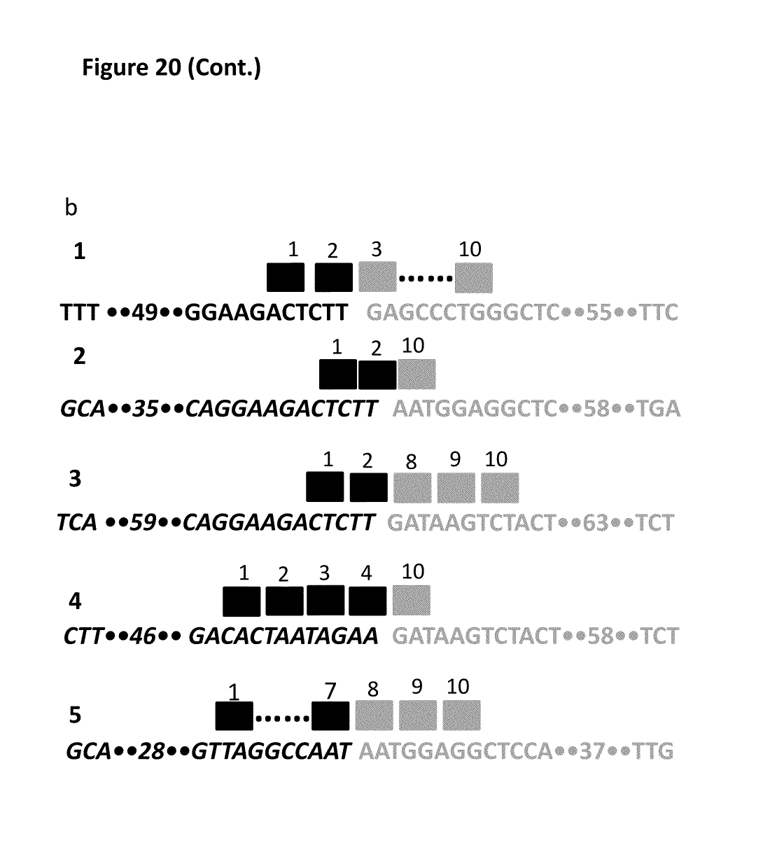

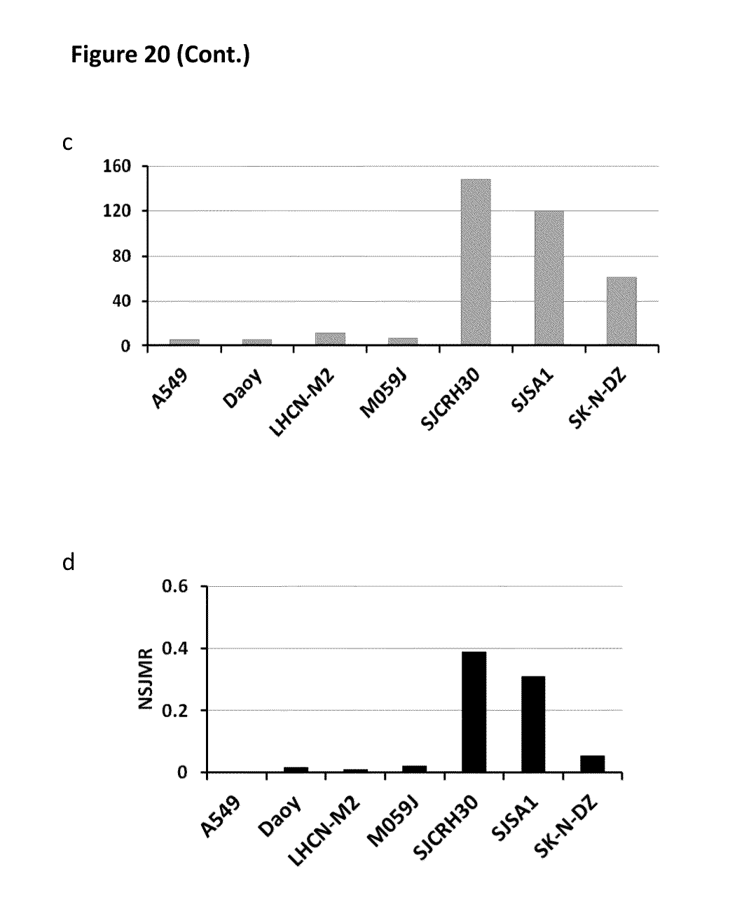

As shown in Table 3, among the top expression recurrent fusion transcripts is from MEG8 and SNORD114-1, which are located in human chromosome 14q32.2 critical region for uniparental disomy of chromosome 14 (UPD(14)) phenotypes and preferentially regulated with other imprinted genes including SNORD114-1 cluster (Charlier, et al. 2001). FIG. 20a shows that a potential inversions or duplications result in reverse orders of MEG8 and SNORNA114-1 and generated SNORD114-1|MEG8 fusion gene structure. We have identified five alternatively-spliced SNORD114-1|MEG8 fusion transcripts from this genetic aberration (FIG. 20b). FIG. 20c shows that the SNORD114-1|MEG8 isoform 3 is highly expressed and 100 folds higher than the isoform 5 (FIG. 20c). The SNORD114-1|MEG8 fusion transcripts have been found in A549, Daoy, LHCN-M2, M059J, SK-N-DZ, SJCRH30 and SJSA1 (FIG. 20d), the last two of which are highly expressed (FIG. 20e). Unlike all fusion genes reported so far, SNORD114-1|MEG8 fusion transcripts are fusion products between snoRNAs and non-coding RNAs and are differentially expressed in the cells (FIG. 20e). This suggests that SNORD114-1|MEG8 fusion transcripts may play some role in cancer developments. It will be important to know the exact functions of SNORD114-1|MEG8 fusion transcripts.

Since this is the first time to report non-coding RNA fusion transcripts, we have performed further analysis of non-coding RNA fusion transcripts. Table 7 has shown that additional fifteen fusion transcripts have been identified, which are involved in seven putative non-coding RNA-RNA fusion genes. It is important for us to understand how these non-coding RNA-RNA fusion transcripts affected the cancer.

As shown in Table 7, from the same genomic regions, we have also detected SNORD114-11|SNORD114-1 inversion fusion transcripts in numbers of cancer cell lines and some normal cell lines. These suggest that this genomic region is prone to genetic instability. Table 7 has shown that additional fifteen fusion transcripts

TABLE 7
Non-coding RNA-RNA fusion transcripts detected in cancer cells lines

| 5′ Genes | 5′ Chr | 5′ End | 3′ Genes | 3′ Chr | 3′ Start |
|---|---|---|---|---|---|
| ncRNA00188 | 17 | 16342728 | SNHG11 | 20 | 37077373 |
| ncRNA00188 | 17 | 16342728 | SNHG7 | 9 | 139619562 |
| ncRNA00188 | 17 | 16344444 | SNHG7 | 9 | 139620868 |
| SNHG3 | 1 | 28835417 | SNHG12 | 1 | 28906099 |
| SNHG3 | 1 | 28834672 | SNHG12 | 1 | 28907158 |
| SNHG3 | 1 | 28843379 | SNHG12 | 1 | 28906493 |
| SNHG3 | 1 | 28834672 | SNORD114-1 | 14 | 101416809 |
| SNHG3 | 1 | 28834672 | SNORD1C | 17 | 74559961 |
| SNHG3 | 1 | 28834672 | SNORD1C | 17 | 74557480 |
| SNORD114-11 | 14 | 101435882 | MEG8 | 14 | 101402336 |
| SNORD114-11 | 14 | 101435061 | MEG8 | 14 | 101402336 |
| SNORD114-11 | 14 | 101435882 | SNORD114-1 | 14 | 101416809 |
| SNORD114-11 | 14 | 101449879 | SNORD114-1 | 14 | 101416809 |
| SNORD114-11 | 14 | 101435882 | SNORD114-1 | 14 | 101420383 |
| SNORD114-11 | 14 | 101435061 | SNORD114-1 | 14 | 101415933 |
| SNORD114-1 | 14 | 101415933 | MEG8 | 14 | 101379858 |
| SNORD114-1 | 14 | 101422286 | MEG8 | 14 | 101379858 |
| SNORD114-1 | 14 | 101417831 | MEG8 | 14 | 101402336 |
| SNORD114-1 | 14 | 101415933 | MEG8 | 14 | 101402336 |
| SNORD114-1 | 14 | 101415933 | MEG8 | 14 | 101365422 |

are involved in seven potential non-coding RNA-RNA fusion genes. It is unclear how these non-coding RNA-RNA fusion transcripts affect the cancer.

Since FIGS. 19 and 20 have suggested that non-coding RNA fusion transcripts may play an important role in cancer developments, we have further analyzed the fusion transcripts and PFGs involved with known non-coding RNA sequences. We have identified 1074 fusion transcripts, which count for 6.5% of the total ECD39 fusion transcripts and are involved in 617 PFGs.

Based on non-coding RNA functions, these fusion transcripts have been classified arbitrarily into 10 subtypes: DANCR (differentiation antagonizing non-protein coding RNA), GASS, MALTA1, miRNAs, snoRNAs, NCRNA, PVT1, SCARNA, SNHGs and TRNA (Gutschner and Diederichs 2012). DANCR (differentiation antagonizing non-protein coding RNA) codes for a 855-base-pair IncRNA, which plays in role in maintaining the undifferentiated state in somatic tissue progenitor cells. GASS (Growth Arrest-Specific 5) has played in role in promoting the apoptosis of prostate cells and growth arrest in human T-lymphocytes (Williams, et al. 2011). MALAT1 (Metastasis-associated lung adenocarcinoma transcript 1) has been implicated in implicates the ncRNA MALAT1 in regulating alternative splicing (Tripathi, et al. 2010). PVT1 is a non-coding RNA oncogene, which is the characteristic lesions associated with Burkitt lymphoma (Ghoussaini, et al. 2008). SCARNA (Small Cajal body-specific RNAs) encodes a class of small nucleolar RNAs that specifically localise to the Cajal body (Enwerem, et al. 2014). All of these RNAs has been suggested to play very important roles in various biological functions (An and Song 2011).

Surprisingly, two miRNAs, MIR17HG and MIR214, have been identified in 20 fusion transcripts. MIR17HG oncogene encodes MIR17-92 cluster, which have a group of at least six miRNAs that may be involved in cell survival, proliferation, differentiation, and angiogenesis (Olive, et al. 2010, Olive, et al. 2013). MIR214 has been found to be involved in intrahepatic cholangiocarcinoma and esophageal squamous cell carcinoma and has been thought to a key hub that controls cancer networks (Penna, et al. 2015). Our analysis has shown that the oncogenic MIR17HG are fused to 9 5′ protein-coding genes while MIR214 have been found to be exclusively spliced to 8 3′ protein-coding genes. Recurrent MIR17HG-GPC5 has been detected in 10 cancer cell lines out of ECD39 cancer cell lines. These data have suggested that MIR17HG and MIR214 have played different roles in regulating these fusion transcripts.

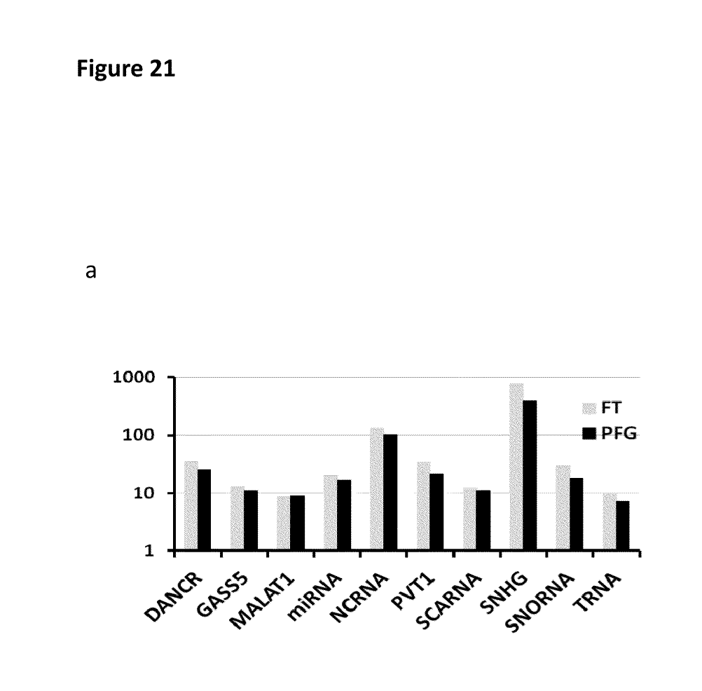

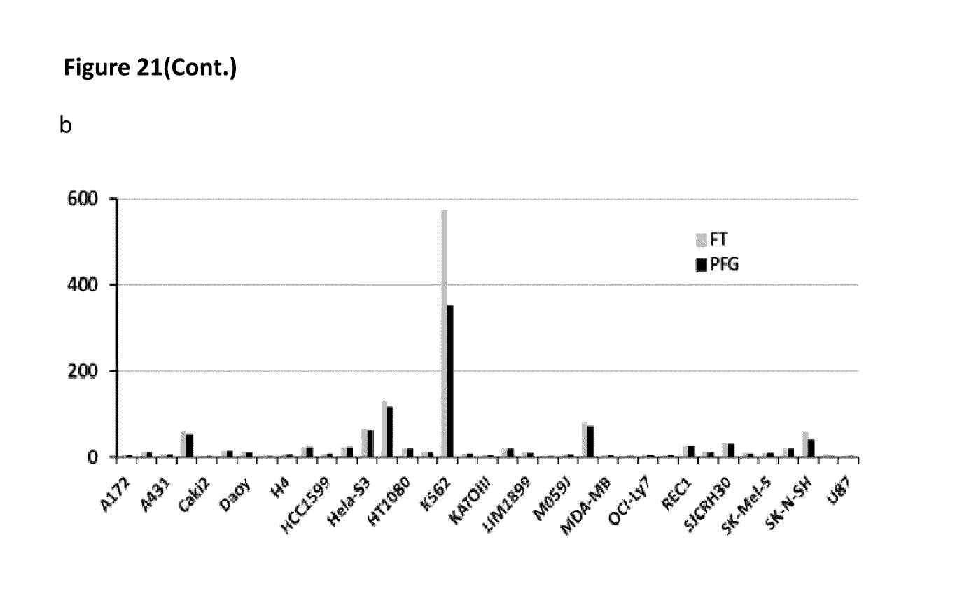

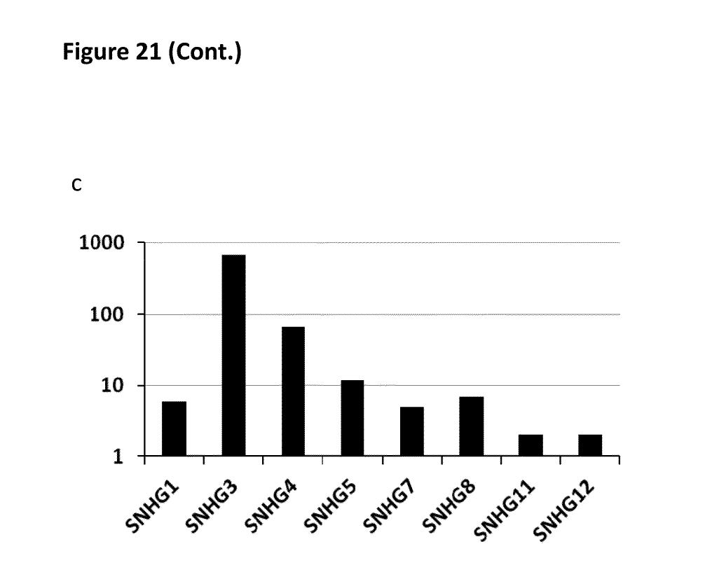

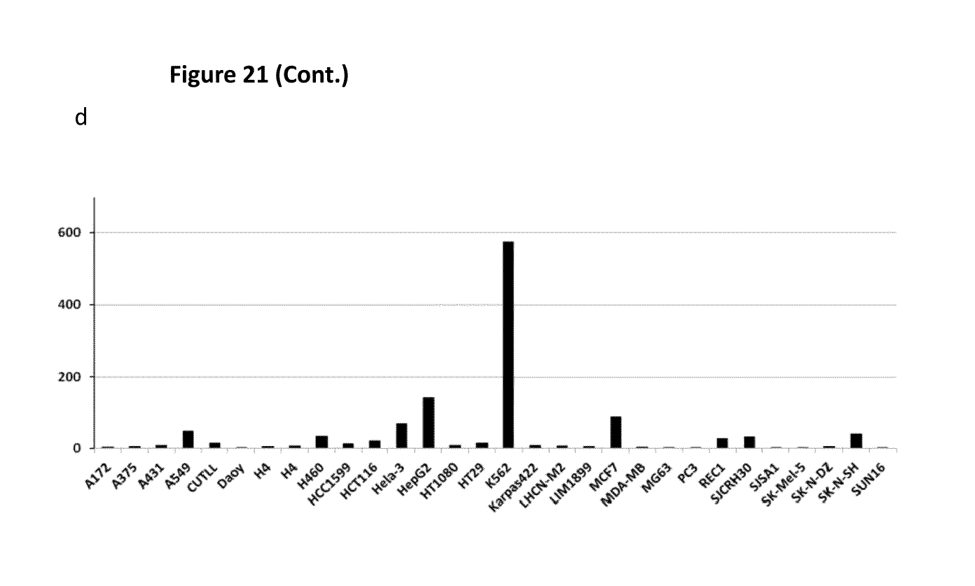

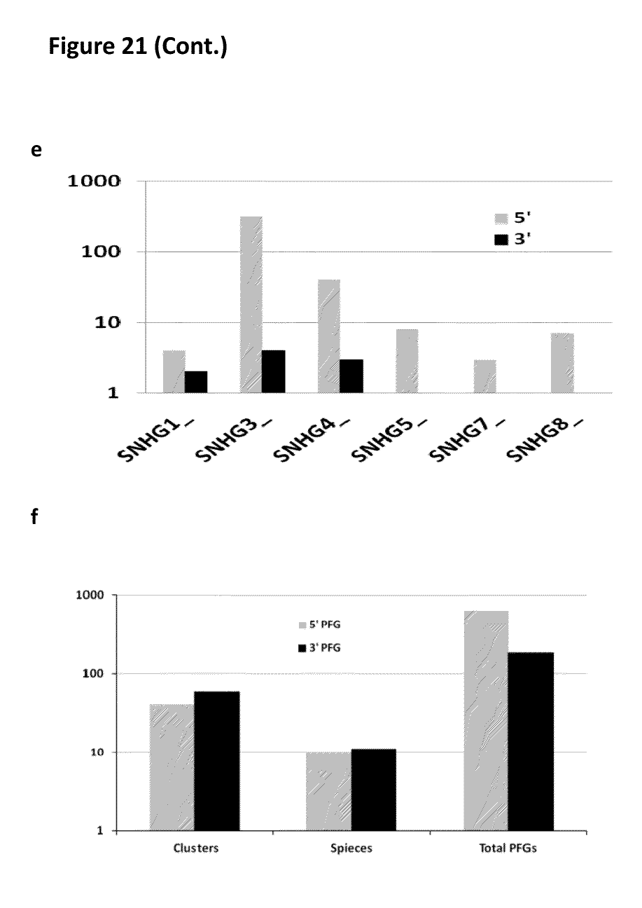

FIG. 21a has shown that the most abundant transcripts involved in non-coding RNAs are transcripts encoding small nucleolar RNA host (SNHG) genes, which count for 73% and 63.7% of the non-coding RNA transcripts and PFGs, respectively. These non-coding RNA fusion transcripts have been detected in 37 out of the 39 cancer cell lines (FIG. 9b). Only U251 and U2OS cell lines have no non-coding RNA fusion transcripts detected so far. This might be due to their smaller RNA-seq datasets and smaller fusion transcript datasets (FIG. 4a).

As shown in FIG. 21b, 574 non-coding RNA fusion transcripts have been detected in K562. In contrast, only 58 fusion transcripts have been observed in SK-N-SH. The difference between the two cancer cell lines is 10 folds even though SK-N-SH has larger RNA-seq read dataset than the K562 one. This suggests that these non-coding RNA fusion transcripts are cancer cell-specific and may play important roles in cancer heterogeneity and development.

As FIG. 21a has shown that the most abundant non-coding RNA fusion transcripts are involved with SNHG genes, we have further analyzed the SNHG fusion transcripts. FIG. 21c has shown that eight SNHG genes are found in fusion transcripts, among which SNHG3 fusion transcripts are the most abundant and count for 87% while the rest 7 SNHG genes count for only 13%. These dominant SNHG3 fusion transcripts are then classified based on the cancer cell lines. FIG. 21d has shown that SNHG3 fusion transcripts have been detected in 30 different cancer lines.

Consistent with results in FIG. 21b, 86% (573 out of 667) of the SNHG3 fusion transcripts have been found in K562. In contrast, only 6.1% (41 of 667) of them are detected in SK-N-SH and are about 14 folds less than that detected in K562. Such a high frequency of SNHG3 sequence being detected in fusion transcripts in K562 cell line strongly suggested a possibility that these fusion transcripts would constitute a natural network, which could be regulated by factors interacting with SNHG3 sequences.

SNHG3 is member of the H/ACA-box class of small nucleolar RNAs (snoRNAs) and is located 9 kb upstream of RCC1 locus coding for regulator of chromosome condensation 1, 5-10% of which are read-through and generated fusion SNHG3 transcripts (Pelczar and Filipowicz 1998).

It has been shown that the SNHG3 gene has been found to interact with a number of chromatin binding proteins/complexes including PRC1, PRC2, JARID1B and SUV39H1 mouse embryonic stem cells (Guttman, et al. 2011). Like most of the SNHG RNA fusion transcripts, >99.99% of SNGH3 sequences are located upstream of the fusion transcripts (FIG. 21e).

Since these non-coding RNA (such as SNHG3) fusion transcripts originate from one cell line, discoveries that sequences from one non-coding RNA gene are translocated to different upstream and/or downstream sequences of different genes raise possibilities that these non-coding RNA fusion transcripts can be regulated at same time by factors that recognize these non-coding RNAs. Therefore, we have proposed that these fusion transcripts by sequences from one gene constitute a natural network, which are different from those interaction networks or networks formed by protein complexes. Here, we have arbitrarily defined a 5′ natural network as sequences from a gene that have been fused to a group of upstream sequences of ≧5 different fusion transcripts. A 3′ natural network has been defined as sequences from a gene or transcriptional unit is added to downstream ≧5 different gene sequences in a cancer cell line. Since this kind of natural network can exist only within a single cell, we, first, have classified fusion transcripts based on the cell line and then classified fusion transcripts based on transcriptional units.

First, we have classified the 3′ natural networks in the cancer cells. Table 8 has shown that fusion transcripts form 3′ natural networks in the different cancer cell lines. The NCBI Aceview's gene names of the complex transcriptional units (annotated ≧2 genes form one transcriptional unit) have been abbreviated. Only the first gene name of the ≧2 gene names will be shown in the tables.

TABLE 8
The 3′ natural networks formed by fusion transcripts

| 5′ Genes | 3′ Genes | 5′ Chr | 3′ Chr | Cancer Cells |
|---|---|---|---|---|
| C17orf70 | ACTG1 | 17 | 17 | A549 |
| HSPG2 | ACTG1 | 1 | 17 | A549 |
| P4HTM | ACTG1 | 3 | 17 | A549 |
| PTPRJ | ACTG1 | 11 | 17 | A549 |
| PUM2 | ACTG1 | 2 | 17 | A549 |
| TSPAN4 | ACTG1 | 11 | 17 | A549 |
| ADAT1 | BCAR1 | 16 | 16 | A549 |
| B4GALT1 | BCAR1 | 9 | 16 | A549 |
| EIF5A | BCAR1 | 17 | 16 | A549 |
| SYNCRIP | BCAR1 | 6 | 16 | A549 |
| ZNRF1 | BCAR1 | 16 | 16 | A549 |
| ARL6IP1 | BCAS3 | 16 | 17 | A549 |
| ASPH | C9orf3 | 8 | 9 | A549 |
| ASPH | C9orf3 | 8 | 9 | A549 |
| ATOH8 | C9orf3 | 2 | 9 | A549 |
| BAHD1 | C9orf3 | 15 | 9 | A549 |
| CALM2 | C9orf3 | 2 | 9 | A549 |
| CARS | C9orf3 | 11 | 9 | A549 |
| CLPTM1 | C9orf3 | 19 | 9 | A549 |
| CYP24A1 | C9orf3 | 20 | 9 | A549 |
| EEF1D | C9orf3 | 8 | 9 | A549 |
| EEF1E1 | C9orf3 | 6 | 9 | A549 |
| FANCC | C9orf3 | 9 | 9 | A549 |
| HNRNPA2B1 | C9orf3 | 7 | 9 | A549 |
| HNRNPA2B1 | C9orf3 | 7 | 9 | A549 |
| HUWE1 | C9orf3 | 23 | 9 | A549 |
| HUWE1 | C9orf3 | 23 | 9 | A549 |
| LOC100288778 | C9orf3 | 12 | 9 | A549 |
| MCM7 | C9orf3 | 7 | 9 | A549 |
| MTA1 | C9orf3 | 14 | 9 | A549 |
| PRR13 | C9orf3 | 12 | 9 | A549 |
| PRR13 | C9orf3 | 12 | 9 | A549 |
| RNASEN | C9orf3 | 5 | 9 | A549 |
| RNASEN | C9orf3 | 5 | 9 | A549 |
| RPL23AP79 | C9orf3 | 19 | 9 | A549 |
| TCF25 | C9orf3 | 16 | 9 | A549 |
| TRAM1 | C9orf3 | 8 | 9 | A549 |
| TSSC4 | C9orf3 | 11 | 9 | A549 |
| TXN | C9orf3 | 9 | 9 | A549 |
| VAV2 | C9orf3 | 9 | 9 | A549 |
| VRK2 | C9orf3 | 2 | 9 | A549 |
| C9orf46 | CHMP1A | 9 | 16 | A549 |
| CPSF6 | CHMP1A | 12 | 16 | A549 |
| ETFA | CHMP1A | 15 | 16 | A549 |
| FUBP1 | CHMP1A | 1 | 16 | A549 |
| LOC146880 | CHMP1A | 17 | 16 | A549 |
| SNX1 | CHMP1A | 15 | 16 | A549 |
| ZNF595 | CHMP1A | 4 | 16 | A549 |
| CALM2 | CTBP1 | 2 | 4 | A549 |
| CALM2 | CTBP1 | 2 | 4 | A549 |
| ILF3 | CTBP1 | 19 | 4 | A549 |
| KIAA1530 | CTBP1 | 4 | 4 | A549 |
| KIAA1530 | CTBP1 | 4 | 4 | A549 |
| NOP14 | CTBP1 | 4 | 4 | A549 |
| SBNO2 | CTBP1 | 19 | 4 | A549 |
| HNRNPH1 | DAZAP1 | 5 | 19 | A549 |
| NFIC | DAZAP1 | 19 | 19 | A549 |
| SBNO2 | DAZAP1 | 19 | 19 | A549 |
| SF3A2 | DAZAP1 | 19 | 19 | A549 |
| STK11 | DAZAP1 | 19 | 19 | A549 |
| ZEB1 | DAZAP1 | 10 | 19 | A549 |
| C9orf3 | GNAS | 9 | 20 | A549 |
| HNRNPK | GNAS | 9 | 20 | A549 |
| KYNU | GNAS | 2 | 20 | A549 |
| MTCP1NB | GNAS | 23 | 20 | A549 |
| SNHG4 | GNAS | 5 | 20 | A549 |
| VAPB | GNAS | 20 | 20 | A549 |
| C17orf56 | MAFK | 17 | 7 | A549 |
| CALM2 | MAFK | 2 | 7 | A549 |
| DEAF1 | MAFK | 11 | 7 | A549 |
| MAD1L1 | MAFK | 7 | 7 | A549 |
| MAD1L1 | MAFK | 7 | 7 | A549 |
| MAD1L1 | MAFK | 7 | 7 | A549 |
| MICALL2 | MAFK | 7 | 7 | A549 |
| SLC7A5 | MAFK | 16 | 7 | A549 |
| UBASH3B | MAFK | 11 | 7 | A549 |
| APP | OVOL2 | 21 | 20 | A549 |
| IDH2 | OVOL2 | 15 | 20 | A549 |
| ncRNA00188 | OVOL2 | 17 | 20 | A549 |
| PAQR5 | OVOL2 | 15 | 20 | A549 |
| TBC1D8 | OVOL2 | 2 | 20 | A549 |
| TBC1D8 | OVOL2 | 2 | 20 | A549 |
| TMEM138 | OVOL2 | 11 | 20 | A549 |
| TXNRD1 | OVOL2 | 12 | 20 | A549 |
| TXNRD1 | OVOL2 | 12 | 20 | A549 |
| COX6A1 | GCN1L1 | 12 | 12 | A549 |
| DCI | GCN1L1 | 16 | 12 | A549 |
| MAN2C1 | GCN1L1 | 15 | 12 | A549 |
| PRPF8 | GCN1L1 | 17 | 12 | A549 |
| PXN | GCN1L1 | 12 | 12 | A549 |
| SBNO1 | GCN1L1 | 12 | 12 | A549 |
| 2-Sep | GCN1L1 | 2 | 12 | A549 |
| TLN2 | GCN1L1 | 15 | 12 | A549 |
| TMEM116 | GCN1L1 | 12 | 12 | A549 |
| TMEM116 | GCN1L1 | 12 | 12 | A549 |
| TRAPPC4 | GCN1L1 | 11 | 12 | A549 |
| UBE3A | GCN1L1 | 15 | 12 | A549 |
| ANKRD11 | SLC7A5 | 16 | 16 | A549 |
| ANKRD11 | SLC7A5 | 16 | 16 | A549 |
| BANP | SLC7A5 | 16 | 16 | A549 |
| BANP | SLC7A5 | 16 | 16 | A549 |
| KIAA0182 | SLC7A5 | 16 | 16 | A549 |
| KLHDC4 | SLC7A5 | 16 | 16 | A549 |
| KLHDC4 | SLC7A5 | 16 | 16 | A549 |
| KLHDC4 | SLC7A5 | 16 | 16 | A549 |
| C7orf44 | SUN1 | 7 | 7 | A549 |
| C7orf50 | SUN1 | 7 | 7 | A549 |
| EIF4EBP2 | SUN1 | 10 | 7 | A549 |
| HEATR2 | SUN1 | 7 | 7 | A549 |
| HNRNPF | SUN1 | 10 | 7 | A549 |
| MICALL2 | SUN1 | 7 | 7 | A549 |
| PRKAR1B | SUN1 | 7 | 7 | A549 |
| PRKAR1B | SUN1 | 7 | 7 | A549 |
| AKT1 | TSPAN4 | 14 | 11 | A549 |
| CALM2 | TSPAN4 | 2 | 11 | A549 |
| CHID1 | TSPAN4 | 11 | 11 | A549 |
| COL5A1 | TSPAN4 | 9 | 11 | A549 |
| EEF1D | TSPAN4 | 8 | 11 | A549 |
| FBF1 | TSPAN4 | 17 | 11 | A549 |
| HNRNPC | TSPAN4 | 14 | 11 | A549 |
| MED13L | TSPAN4 | 12 | 11 | A549 |
| PPP1R12C | TSPAN4 | 19 | 11 | A549 |
| PPP6R2 | TSPAN4 | 22 | 11 | A549 |
| RGS20 | TSPAN4 | 8 | 11 | A549 |
| SETD8 | TSPAN4 | 12 | 11 | A549 |
| SHANK3 | TSPAN4 | 22 | 11 | A549 |
| TOB1 | TSPAN4 | 17 | 11 | A549 |
| UCKL1 | TSPAN4 | 20 | 11 | A549 |
| DDX5 | UBC | 17 | 12 | A549 |
| KRT80 | UBC | 12 | 12 | A549 |
| NCOR2 | UBC | 12 | 12 | A549 |
| NCOR2 | UBC | 12 | 12 | A549 |
| NCOR2 | UBC | 12 | 12 | A549 |
| NCOR2 | UBC | 12 | 12 | A549 |
| ORAOV1 | UBC | 11 | 12 | A549 |
| UHRF1BP1L | UBC | 12 | 12 | A549 |
| ZNRD1 | UBC | 6 | 12 | A549 |
| ABCC3 | ZNF598 | 17 | 16 | A549 |
| E4F1 | ZNF598 | 16 | 16 | A549 |
| EEF1D | ZNF598 | 8 | 16 | A549 |
| TECPR1 | ZNF598 | 7 | 16 | A549 |
| SNHG3 | ZNF638 | 1 | 2 | A549 |
| ACAD10 | GNAS | 12 | 20 | CUTLL |
| GEN1 | GNAS | 2 | 20 | CUTLL |
| HNRNPH1 | GNAS | 5 | 20 | CUTLL |
| MYL6B | GNAS | 12 | 20 | CUTLL |
| SLMO2 | GNAS | 20 | 20 | CUTLL |
| HNRNPF | DAZAP1 | 10 | 19 | Hela-3 |
| HNRNPF | DAZAP1 | 10 | 19 | Hela-3 |
| NDUFS7 | DAZAP1 | 19 | 19 | Hela-3 |
| NFIC | DAZAP1 | 19 | 19 | Hela-3 |
| PPP1R12C | DAZAP1 | 19 | 19 | Hela-3 |
| PPP1R12C | DAZAP1 | 19 | 19 | Hela-3 |
| RPL22 | DAZAP1 | 1 | 19 | Hela-3 |
| SBNO2 | DAZAP1 | 19 | 19 | Hela-3 |
| SULT1A1 | DAZAP1 | 16 | 19 | Hela-3 |
| ANKRD11 | FAM156B | 16 | 23 | Hela-3 |
| FAM156A | FAM156B | 23 | 23 | Hela-3 |
| KANK2 | FAM156B | 19 | 23 | Hela-3 |
| RASA4P | FAM156B | 7 | 23 | Hela-3 |
| SLC6A15 | FAM156B | 12 | 23 | Hela-3 |
| PLEKHB2 | FAM168B | 2 | 2 | Hela-3 |
| ASAP1 | GNAS | 8 | 20 | Hela-3 |
| BRCA1P1 | GNAS | 17 | 20 | Hela-3 |
| CBX5 | GNAS | 12 | 20 | Hela-3 |
| GEN1 | GNAS | 2 | 20 | Hela-3 |
| HUWE1 | GNAS | 23 | 20 | Hela-3 |
| KIAA0182 | GNAS | 16 | 20 | Hela-3 |
| KYNU | GNAS | 2 | 20 | Hela-3 |
| SLMO2 | GNAS | 20 | 20 | Hela-3 |
| SNHG3 | GNAS | 1 | 20 | Hela-3 |
| TP53 | GNAS | 17 | 20 | Hela-3 |
| C5 | FN1 | 9 | 2 | HepG2 |
| HNRNPH1 | FN1 | 5 | 2 | HepG2 |
| NAA35 | FN1 | 9 | 2 | HepG2 |
| RPL31 | FN1 | 2 | 2 | HepG2 |
| RPL31 | FN1 | 2 | 2 | HepG2 |
| SNHG3 | FN1 | 1 | 2 | HepG2 |
| SNHG3 | FN1 | 1 | 2 | HepG2 |
| TTC15 | FN1 | 2 | 2 | HepG2 |
| ARF1 | GNAS | 1 | 20 | HepG2 |
| B2M | GNAS | 15 | 20 | HepG2 |
| CBX5 | GNAS | 12 | 20 | HepG2 |
| EEF1D | GNAS | 8 | 20 | HepG2 |
| HNRNPH1 | GNAS | 5 | 20 | HepG2 |
| KPNA6 | GNAS | 1 | 20 | HepG2 |
| MGA | GNAS | 15 | 20 | HepG2 |
| SLMO2 | GNAS | 20 | 20 | HepG2 |
| STAG2 | GNAS | 23 | 20 | HepG2 |
| ANKRD11 | OVOL2 | 16 | 20 | HepG2 |
| APP | OVOL2 | 21 | 20 | HepG2 |
| JUB | OVOL2 | 14 | 20 | HepG2 |
| TASP1 | OVOL2 | 20 | 20 | HepG2 |
| ZNF133 | OVOL2 | 20 | 20 | HepG2 |
| ZNF519 | OVOL2 | 18 | 20 | HepG2 |
| CHD6 | GNAS | 20 | 20 | HT29 |
| CORO7 | GNAS | 16 | 20 | HT29 |
| DNAJB6 | GNAS | 7 | 20 | HT29 |
| RPL12P27 | GNAS | 10 | 20 | HT29 |
| RSU1 | GNAS | 10 | 20 | HT29 |
| C7orf50 | MAD1L1 | 7 | 7 | HT29 |
| NFE2L3 | MAD1L1 | 7 | 7 | HT29 |
| TTYH3 | MAD1L1 | 7 | 7 | HT29 |
| UBAP1 | MAD1L1 | 9 | 7 | HT29 |
| ZNF766 | MAD1L1 | 19 | 7 | HT29 |
| HNRNPH1 | CANX | 5 | 5 | K562 |
| MAPK9 | CANX | 5 | 5 | K562 |
| PPFIA1 | CANX | 11 | 5 | K562 |
| SNHG3 | CANX | 1 | 5 | K562 |
| SNHG3 | CANX | 1 | 5 | K562 |
| SNHG3 | CANX | 1 | 5 | K562 |
| SQSTM1 | CANX | 5 | 5 | K562 |
| SQSTM1 | CANX | 5 | 5 | K562 |
| KIAA1530 | CTBP1 | 4 | 4 | K562 |
| LOC100129917 | CTBP1 | 4 | 4 | K562 |
| MAEA | CTBP1 | 4 | 4 | K562 |
| OAZ1 | CTBP1 | 19 | 4 | K562 |
| PCGF3 | CTBP1 | 4 | 4 | K562 |
| PCGF3 | CTBP1 | 4 | 4 | K562 |
| SPON2 | CTBP1 | 4 | 4 | K562 |
| ASH1L | DAP3 | 1 | 1 | K562 |
| C14orf156 | DAP3 | 14 | 1 | K562 |
| C14orf156 | DAP3 | 14 | 1 | K562 |
| GON4L | DAP3 | 1 | 1 | K562 |
| GON4L | DAP3 | 1 | 1 | K562 |
| SNHG3 | DAP3 | 1 | 1 | K562 |
| SNHG3 | DAP3 | 1 | 1 | K562 |
| SNHG3 | DAP3 | 1 | 1 | K562 |
| SNHG3 | DAP3 | 1 | 1 | K562 |
| SSR2 | DAP3 | 1 | 1 | K562 |
| IVNS1ABP | EIF3E | 1 | 8 | K562 |
| SNHG3 | EIF3E | 1 | 8 | K562 |
| ST3GAL1 | EIF3E | 8 | 8 | K562 |
| ST3GAL1 | EIF3E | 8 | 8 | K562 |
| TTC35 | EIF3E | 8 | 8 | K562 |
| XRCC4 | EIF3E | 5 | 8 | K562 |
| CHCHD3 | FAF1 | 7 | 1 | K562 |
| CHCHD3 | FAF1 | 7 | 1 | K562 |
| KIAA0114 | FAF1 | 4 | 1 | K562 |
| MIR17HG | FAF1 | 13 | 1 | K562 |
| OSBPL9 | FAF1 | 1 | 1 | K562 |
| OSBPL9 | FAF1 | 1 | 1 | K562 |
| RNF11 | FAF1 | 1 | 1 | K562 |
| RNF11 | FAF1 | 1 | 1 | K562 |
| RNF11 | FAF1 | 1 | 1 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG5 | FAF1 | 6 | 1 | K562 |
| SNORD1C | FAF1 | 17 | 1 | K562 |
| TM2D1 | FAF1 | 1 | 1 | K562 |
| C10orf18 | GDI2 | 10 | 10 | K562 |
| C10orf18 | GDI2 | 10 | 10 | K562 |
| NET1 | GDI2 | 10 | 10 | K562 |
| PFKFB3 | GDI2 | 10 | 10 | K562 |
| POLR2D | GDI2 | 2 | 10 | K562 |
| RBM17 | GDI2 | 10 | 10 | K562 |
| SNHG3 | GDI2 | 1 | 10 | K562 |
| SNHG3 | GDI2 | 1 | 10 | K562 |
| WDR37 | GDI2 | 10 | 10 | K562 |
| ABO | GNAS | 9 | 20 | K562 |
| ACAD10 | GNAS | 12 | 20 | K562 |
| ARHGEF2 | GNAS | 1 | 20 | K562 |
| BCOR | GNAS | 23 | 20 | K562 |
| FAM49B | GNAS | 8 | 20 | K562 |
| FAM60A | GNAS | 12 | 20 | K562 |
| HDLBP | GNAS | 2 | 20 | K562 |
| ITCH | GNAS | 20 | 20 | K562 |
| KIAA0182 | GNAS | 16 | 20 | K562 |
| MIPOL1 | GNAS | 14 | 20 | K562 |
| NFYC | GNAS | 1 | 20 | K562 |
| NOC4L | GNAS | 12 | 20 | K562 |
| SHB | GNAS | 9 | 20 | K562 |
| SNHG4 | GNAS | 5 | 20 | K562 |
| TYMS | GNAS | 18 | 20 | K562 |
| ROD1 | KIAA0368 | 9 | 9 | K562 |
| SUSD1 | KIAA0368 | 9 | 9 | K562 |
| SUSD1 | KIAA0368 | 9 | 9 | K562 |
| TXN | KIAA0368 | 9 | 9 | K562 |
| UGCG | KIAA0368 | 9 | 9 | K562 |
| UGCG | KIAA0368 | 9 | 9 | K562 |
| VPS13A | KIAA0368 | 9 | 9 | K562 |
| RCSD1 | PDS5A | 1 | 4 | K562 |
| SNHG3 | PDS5A | 1 | 4 | K562 |
| TMEM165 | PDS5A | 4 | 4 | K562 |
| UBE2K | PDS5A | 4 | 4 | K562 |
| UBE2K | PDS5A | 4 | 4 | K562 |
| USP34 | PDS5A | 2 | 4 | K562 |
| ARL4A | PHF14 | 7 | 7 | K562 |
| KIAA0114 | PHF14 | 4 | 7 | K562 |
| ncRNA00188 | PHF14 | 17 | 7 | K562 |
| ncRNA00188 | PHF14 | 17 | 7 | K562 |
| NDUFA4 | PHF14 | 7 | 7 | K562 |
| SNHG3 | PHF14 | 1 | 7 | K562 |
| VWDE | PHF14 | 7 | 7 | K562 |
| VWDE | PHF14 | 7 | 7 | K562 |
| C11orf73 | PICALM | 11 | 11 | K562 |
| C11orf73 | PICALM | 11 | 11 | K562 |
| COPB2 | PICALM | 3 | 11 | K562 |
| COPB2 | PICALM | 3 | 11 | K562 |
| EED | PICALM | 11 | 11 | K562 |
| FDXACB1 | PICALM | 11 | 11 | K562 |
| KIAA0114 | PICALM | 4 | 11 | K562 |
| KIF2A | PICALM | 5 | 11 | K562 |
| RPS20 | PICALM | 8 | 11 | K562 |
| RPS20 | PICALM | 8 | 11 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG4 | PICALM | 5 | 11 | K562 |
| SNHG4 | PICALM | 5 | 11 | K562 |
| TAF1 | PICALM | 11 | 11 | K562 |
| TAF1 | PICALM | 11 | 11 | K562 |
| TMEM126B | PICALM | 11 | 11 | K562 |
| ZNF33B | PICALM | 10 | 11 | K562 |
| AGPAT5 | PRKCB | 8 | 16 | K562 |
| AGPAT5 | PRKCB | 8 | 16 | K562 |
| C15orf26 | PRKCB | 15 | 16 | K562 |
| C15orf26 | PRKCB | 15 | 16 | K562 |
| KIAA0114 | PRKCB | 4 | 16 | K562 |
| KIAA0114 | PRKCB | 4 | 16 | K562 |
| SNHG3 | PRKCB | 1 | 16 | K562 |
| SNHG3 | PRKCB | 1 | 16 | K562 |
| ACOX3 | PSMB1 | 4 | 6 | K562 |
| CTBP2 | PSMB1 | 10 | 6 | K562 |
| HGS | PSMB1 | 17 | 6 | K562 |
| MLL5 | PSMB1 | 7 | 6 | K562 |
| PAOX | PSMB1 | 10 | 6 | K562 |
| WDR27 | PSMB1 | 6 | 6 | K562 |
| ZDHHC14 | PSMB1 | 6 | 6 | K562 |
| TWSG1 | RALBP1 | 18 | 18 | K562 |
| ARHGEF18 | RANBP1 | 19 | 22 | K562 |
| BOP1 | RANBP1 | 8 | 22 | K562 |
| C22orf25 | RANBP1 | 22 | 22 | K562 |
| C22orf25 | RANBP1 | 22 | 22 | K562 |
| EIF5A | RANBP1 | 17 | 22 | K562 |
| KLHL22 | RANBP1 | 22 | 22 | K562 |
| MED15 | RANBP1 | 22 | 22 | K562 |
| RPS8 | RANBP1 | 1 | 22 | K562 |
| RPS8 | RANBP1 | 1 | 22 | K562 |
| SNHG3 | RANBP1 | 1 | 22 | K562 |
| SNHG3 | RANBP1 | 1 | 22 | K562 |
| VRK2 | RANBP1 | 2 | 22 | K562 |
| WHSC2 | RANBP1 | 4 | 22 | K562 |
| WHSC2 | RANBP1 | 4 | 22 | K562 |
| VMAC | RANBP3 | 19 | 19 | K562 |
| HIVEP1 | RANBP9 | 6 | 6 | K562 |
| GNL3 | RNF149 | 3 | 2 | K562 |
| RPL10 | RNF149 | 23 | 2 | K562 |
| RPL17A | RNF149 | 9 | 2 | K562 |
| RPL27A | RNF149 | 11 | 2 | K562 |
| RPL3 | RNF149 | 22 | 2 | K562 |
| SCARNA17 | RNF149 | 18 | 2 | K562 |
| SNHG3 | RNF149 | 1 | 2 | K562 |
| SNHG3 | RNF149 | 1 | 2 | K562 |
| LARP4 | GCN1L1 | 12 | 12 | K562 |
| NOP2 | GCN1L1 | 12 | 12 | K562 |
| NUP210 | GCN1L1 | 3 | 12 | K562 |
| SBNO1 | GCN1L1 | 12 | 12 | K562 |
| TMEM138 | GCN1L1 | 11 | 12 | K562 |
| HERC1 | RPS27A | 15 | 2 | K562 |
| KLF1 | RPS27A | 19 | 2 | K562 |
| RPL27A | RPS27A | 11 | 2 | K562 |
| RPL27A | RPS27A | 11 | 2 | K562 |
| RPS3 | RPS27A | 11 | 2 | K562 |
| SNHG4 | RPS27A | 5 | 2 | K562 |
| SNHG4 | RPS27A | 5 | 2 | K562 |
| SPTBN1 | RPS27A | 2 | 2 | K562 |
| SRP9 | RPS27A | 1 | 2 | K562 |
| TOMM20 | RPS27A | 1 | 2 | K562 |
| ncRNA00188 | RPS3 | 17 | 11 | K562 |
| ncRNA00188 | RPS3 | 17 | 11 | K562 |
| RPL27A | RPS3 | 11 | 11 | K562 |
| SNHG3 | RPS3 | 1 | 11 | K562 |
| SNHG3 | RPS3 | 1 | 11 | K562 |
| AFF1 | SIKE1 | 4 | 1 | K562 |
| AK2 | SIKE1 | 1 | 1 | K562 |
| BAZ1A | SIKE1 | 14 | 1 | K562 |
| CAPZA1 | SIKE1 | 1 | 1 | K562 |
| SNHG3 | SIKE1 | 1 | 1 | K562 |
| SNHG3 | SIKE1 | 1 | 1 | K562 |
| TRIM33 | SIKE1 | 1 | 1 | K562 |
| BAHD1 | UBB | 15 | 17 | K562 |
| CAPZA1 | UBB | 1 | 17 | K562 |
| CAPZA2 | UBB | 7 | 17 | K562 |
| EEF1A1 | UBB | 6 | 17 | K562 |
| IL23R | UBB | 1 | 17 | K562 |
| LARP4 | UBB | 12 | 17 | K562 |
| MYL6B | UBB | 12 | 17 | K562 |
| PI4KA | UBB | 22 | 17 | K562 |
| RAI1 | UBB | 17 | 17 | K562 |
| RASA4P | UBB | 7 | 17 | K562 |
| RPL15 | UBB | 3 | 17 | K562 |
| RPL15 | UBB | 3 | 17 | K562 |
| RPS3 | UBB | 11 | 7 | K562 |
| SNHG3 | UBB | 1 | 17 | K562 |
| CRAMP1L | UBE2I | 16 | 16 | K562 |
| LMF1 | UBE2I | 16 | 16 | K562 |
| SNHG3 | UBE2I | 1 | 16 | K562 |
| SNHG3 | UBE2I | 1 | 16 | K562 |
| SOLH | UBE2I | 16 | 16 | K562 |
| SOLH | UBE2I | 16 | 16 | K562 |
| WHSC1 | UBE2I | 4 | 16 | K562 |
| ABCC5 | STAMBP | 3 | 2 | LHCN-M2 |
| ADAMTS2 | STAMBP | 5 | 2 | LHCN-M2 |
| ANKRD11 | STAMBP | 16 | 2 | LHCN-M2 |
| BCL2L11 | STAMBP | 2 | 2 | LHCN-M2 |
| BLCAP | STAMBP | 20 | 2 | LHCN-M2 |
| C7orf13 | STAMBP | 7 | 2 | LHCN-M2 |
| CABLES1 | STAMBP | 18 | 2 | LHCN-M2 |
| CALM2 | STAMBP | 2 | 2 | LHCN-M2 |
| CAPN2 | STAMBP | 1 | 2 | LHCN-M2 |
| CCDC46 | STAMBP | 17 | 2 | LHCN-M2 |
| CCDC55 | STAMBP | 17 | 2 | LHCN-M2 |
| CHFR | STAMBP | 12 | 2 | LHCN-M2 |
| CLK3 | STAMBP | 15 | 2 | LHCN-M2 |
| COL3A1 | STAMBP | 2 | 2 | LHCN-M2 |
| CWC22 | STAMBP | 2 | 2 | LHCN-M2 |
| DECR1 | STAMBP | 8 | 2 | LHCN-M2 |
| ERP44 | STAMBP | 9 | 2 | LHCN-M2 |
| FRYL | STAMBP | 4 | 2 | LHCN-M2 |
| FRYL | STAMBP | 4 | 2 | LHCN-M2 |
| GAS6 | STAMBP | 13 | 2 | LHCN-M2 |
| HDAC5 | STAMBP | 17 | 2 | LHCN-M2 |
| HNRNPK | STAMBP | 9 | 2 | LHCN-M2 |
| KDM6A | STAMBP | 23 | 2 | LHCN-M2 |
| KLF5 | STAMBP | 13 | 2 | LHCN-M2 |
| KLHL29 | STAMBP | 2 | 2 | LHCN-M2 |
| LDLRAD3 | STAMBP | 11 | 2 | LHCN-M2 |
| LOC100129917 | STAMBP | 4 | 2 | LHCN-M2 |
| LRRC28 | STAMBP | 15 | 2 | LHCN-M2 |
| LSP1 | STAMBP | 11 | 2 | LHCN-M2 |
| MAD1L1 | STAMBP | 7 | 2 | LHCN-M2 |
| MBD2 | STAMBP | 18 | 2 | LHCN-M2 |
| MED8 | STAMBP | 1 | 2 | LHCN-M2 |
| MTMR3 | STAMBP | 22 | 2 | LHCN-M2 |
| NAPG | STAMBP | 18 | 2 | LHCN-M2 |
| PCDH9 | STAMBP | 13 | 2 | LHCN-M2 |
| PCDHG | STAMBP | 5 | 2 | LHCN-M2 |
| PCDHG | STAMBP | 5 | 2 | LHCN-M2 |
| PICALM | STAMBP | 11 | 2 | LHCN-M2 |
| POFUT2 | STAMBP | 21 | 2 | LHCN-M2 |
| PRMT2 | STAMBP | 21 | 2 | LHCN-M2 |
| PRMT2 | STAMBP | 21 | 2 | LHCN-M2 |
| RELT | STAMBP | 11 | 2 | LHCN-M2 |
| RGS20 | STAMBP | 8 | 2 | LHCN-M2 |
| RUNX1 | STAMBP | 21 | 2 | LHCN-M2 |
| 11-Sep | STAMBP | 4 | 2 | LHCN-M2 |
| SLC38A10 | STAMBP | 17 | 2 | LHCN-M2 |
| TMEM87B | STAMBP | 2 | 2 | LHCN-M2 |
| TSPAN3 | STAMBP | 15 | 2 | LHCN-M2 |
| TUBA1A | STAMBP | 12 | 2 | LHCN-M2 |
| UAP1 | STAMBP | 1 | 2 | LHCN-M2 |
| UBE3C | STAMBP | 7 | 2 | LHCN-M2 |
| WDR37 | STAMBP | 10 | 2 | LHCN-M2 |
| WHSC2 | STAMBP | 4 | 2 | LHCN-M2 |
| XPO4 | STAMBP | 13 | 2 | LHCN-M2 |
| ZFP106 | STAMBP | 15 | 2 | LHCN-M2 |
| ZNF24 | STAMBP | 18 | 2 | LHCN-M2 |
| ZNF556 | STAMBP | 19 | 2 | LHCN-M2 |
| ZNF571 | STAMBP | 19 | 2 | LHCN-M2 |
| ZNF702P | STAMBP | 19 | 2 | LHCN-M2 |
| CAPRIN1 | CHMP1A | 11 | 16 | MCF7 |
| CYP24A1 | CHMP1A | 20 | 16 | MCF7 |
| DCP1B | CHMP1A | 12 | 16 | MCF7 |
| ING3 | CHMP1A | 7 | 16 | MCF7 |
| LMBR1 | CHMP1A | 7 | 16 | MCF7 |
| POLD3 | CHMP1A | 11 | 16 | MCF7 |
| POLD3 | CHMP1A | 11 | 16 | MCF7 |
| RBL2 | CHMP1A | 16 | 16 | MCF7 |
| ZNF286A | CHMP1A | 17 | 16 | MCF7 |
| ZNF519 | CHMP1A | 18 | 16 | MCF7 |
| CCDC57 | CHMP4B | 17 | 20 | MCF7 |
| RALY | CHMP4B | 20 | 20 | MCF7 |
| DDX5 | GNAI3 | 17 | 1 | MCF7 |
| NR2F2 | GNAI3 | 15 | 1 | MCF7 |
| SYCP2 | GNAI3 | 20 | 1 | MCF7 |
| TANC2 | GNAI3 | 17 | 1 | MCF7 |
| TNS3 | GNAI3 | 7 | 1 | MCF7 |
| CTBP2 | GNAS | 10 | 20 | MCF7 |
| EEF1D | GNAS | 8 | 20 | MCF7 |
| KIAA0114 | GNAS | 4 | 20 | MCF7 |
| MGA | GNAS | 15 | 20 | MCF7 |
| NCOA3 | GNAS | 20 | 20 | MCF7 |
| SNHG3 | GNAS | 1 | 20 | MCF7 |
| TYMS | GNAS | 18 | 20 | MCF7 |
| YWHAE | GNAS | 17 | 20 | MCF7 |
| ATP5I | ZNF595 | 4 | 4 | MCF7 |
| HSPD1 | ZNF595 | 2 | 4 | MCF7 |
| IKBKAP | ZNF595 | 9 | 4 | MCF7 |
| TOM1L2 | ZNF595 | 17 | 4 | MCF7 |
| TRMT2B | ZNF595 | 23 | 4 | MCF7 |
| FSTL5 | MRPL21 | 4 | 11 | SK-Mel-5 |
| ncRNA00188 | MRPL21 | 17 | 11 | SK-Mel-5 |
| NOP56 | MRPL21 | 20 | 11 | SK-Mel-5 |
| RAB38 | MRPL21 | 11 | 11 | SK-Mel-5 |
| TUBD1 | MRPL21 | 17 | 11 | SK-Mel-5 |
| ATP6V1G2 | MRPL52 | 6 | 14 | SK-Mel-5 |
| ASPH | CHMP1A | 8 | 16 | SK-N-DZ |
| CCDC64 | CHMP1A | 12 | 16 | SK-N-DZ |
| HECTD2 | CHMP1A | 10 | 16 | SK-N-DZ |
| ISCA1 | CHMP1A | 9 | 16 | SK-N-DZ |
| RIMBP2 | CHMP1A | 12 | 16 | SK-N-DZ |
| TMEM165 | CHMP1A | 4 | 16 | SK-N-DZ |
| ZNF726 | CHMP1A | 19 | 16 | SK-N-DZ |
| ZNF738 | CHMP1A | 19 | 16 | SK-N-DZ |
| ATP5I | DDX1 | 4 | 2 | SK-N-DZ |
| DCAKD | DDX1 | 17 | 2 | SK-N-DZ |
| EIF3A | DDX1 | 10 | 2 | SK-N-DZ |
| MANEA | DDX1 | 6 | 2 | SK-N-DZ |
| MED28 | DDX1 | 4 | 2 | SK-N-DZ |
| NBAS | DDX1 | 2 | 2 | SK-N-DZ |
| NBAS | DDX1 | 2 | 2 | SK-N-DZ |
| RPS12 | DDX1 | 6 | 2 | SK-N-DZ |
| SRRM1 | DDX1 | 1 | 2 | SK-N-DZ |
| XPO5 | DDX1 | 6 | 2 | SK-N-DZ |
| ZDBF2 | DDX1 | 2 | 2 | SK-N-DZ |
| CHCHD3 | GNAS | 7 | 20 | SK-N-DZ |
| HIPK3 | GNAS | 11 | 20 | SK-N-DZ |
| KDM2A | GNAS | 11 | 20 | SK-N-DZ |
| P4HTM | GNAS | 3 | 20 | SK-N-DZ |
| PLEKHO2 | GNAS | 15 | 20 | SK-N-DZ |
| SERINC3 | GNAS | 20 | 20 | SK-N-DZ |
| STAG2 | GNAS | 23 | 20 | SK-N-DZ |
| FAM165B | NUP107 | 21 | 12 | SK-N-DZ |
| PHF3 | NUP107 | 6 | 12 | SK-N-DZ |
| RAP1B | NUP107 | 12 | 12 | SK-N-DZ |
| SLC35E3 | NUP107 | 12 | 12 | SK-N-DZ |
| TCP1 | NUP107 | 6 | 12 | SK-N-DZ |
| ADAM10 | ANXA2 | 15 | 15 | SK-N-DZ |
| LOC642776 | ANXA2 | 23 | 15 | SK-N-DZ |
| NPTN | ANXA2 | 15 | 15 | SK-N-DZ |
| TUBB6 | ANXA2 | 18 | 15 | SK-N-DZ |
| YWHAH | ANXA2 | 22 | 15 | SK-N-DZ |
| PROSC | FAM120B | 8 | 6 | SK-N-DZ |
| ZNF519 | FAM120B | 18 | 6 | SK-N-DZ |
| ARHGAP39 | FAM156B | 8 | 23 | SK-N-DZ |
| EXD3 | FAM156B | 9 | 23 | SK-N-DZ |
| KANK2 | FAM156B | 19 | 23 | SK-N-DZ |
| TGM2 | FAM156B | 20 | 23 | SK-N-DZ |
| ANAPC16 | GNAS | 10 | 20 | SK-N-DZ |
| APBB2 | GNAS | 4 | 20 | SK-N-DZ |
| ARHGEF10 | GNAS | 8 | 20 | SK-N-DZ |
| ASAP1 | GNAS | 8 | 20 | SK-N-DZ |
| BRCA1P1 | GNAS | 17 | 20 | SK-N-DZ |
| CCAR1 | GNAS | 10 | 20 | SK-N-DZ |
| CCDC101 | GNAS | 16 | 20 | SK-N-DZ |
| FAM119B | GNAS | 12 | 20 | SK-N-DZ |
| ITCH | GNAS | 20 | 20 | SK-N-DZ |
| NAP1L1 | GNAS | 12 | 20 | SK-N-DZ |
| RBM14 | GNAS | 11 | 20 | SK-N-DZ |
| RPL31 | GNAS | 2 | 20 | SK-N-DZ |
| SFRS18 | GNAS | 6 | 20 | SK-N-DZ |
| TCF4 | GNAS | 18 | 20 | SK-N-DZ |
| TRAF3 | GNAS | 14 | 20 | SK-N-DZ |
| VAPB | GNAS | 20 | 20 | SK-N-DZ |
| ZMYND8 | GNAS | 20 | 20 | SK-N-DZ |
| C7orf50 | SUN1 | 7 | 7 | SK-N-DZ |
| HEATR2 | SUN1 | 7 | 7 | SK-N-DZ |
| MAD1L1 | SUN1 | 7 | 7 | SK-N-DZ |
| PRKAR1B | SUN1 | 7 | 7 | SK-N-DZ |
| PRKAR1B | SUN1 | 7 | 7 | SK-N-DZ |
| TRA2A | SUN1 | 7 | 7 | SK-N-DZ |

Table 9 shows the lists of 5′ networks of fusion transcripts.

TABLE 9
Identification of 5′ natural networks of the fusion
transcripts in different cancer cell lines. Gene names
have been abbreviated to reduce space. If the complex
gene names adopted by NCBI's Aceview contain two more
names connected by “and”, we have used the first
gene name as Gene IDs.
The 5′ natural networks formed by fusion transcripts.

| 5′ Genes | 3′ Genes | 5′ Chr | 3′ Chr | Cancer Cells |
|---|---|---|---|---|
| ABCC3 | KRT8 | 17 | 12 | A549 |
| ABCC3 | SDCCAG3 | 17 | 9 | A549 |
| ABCC3 | 2-Sep | 17 | 2 | A549 |
| ABCC3 | TBCD | 17 | 17 | A549 |
| ABCC3 | ZNF598 | 17 | 16 | A549 |
| ASPH | C9orf3 | 8 | 9 | A549 |
| ASPH | C9orf3 | 8 | 9 | A549 |
| ASPH | FAM120B | 8 | 6 | A549 |
| ASPH | MTCP1NB | 8 | 23 | A549 |
| ASPH | YLPM1 | 8 | 14 | A549 |
| CALM2 | ANKMY1 | 2 | 2 | A549 |
| CALM2 | C9orf3 | 2 | 9 | A549 |
| CALM2 | CRIM1 | 2 | 2 | A549 |
| CALM2 | CTBP1 | 2 | 4 | A549 |
| CALM2 | CTBP1 | 2 | 4 | A549 |
| CALM2 | GNA11 | 2 | 19 | A549 |
| CALM2 | MAFK | 2 | 7 | A549 |
| CALM2 | SBNO2 | 2 | 19 | A549 |
| CALM2 | TSPAN4 | 2 | 11 | A549 |
| CALM2 | TTC7A | 2 | 2 | A549 |
| CALM2 | ZDHHC7 | 2 | 16 | A549 |
| CPSF4 | FSCN1 | 7 | 7 | A549 |
| CPSF6 | CHMP1A | 12 | 16 | A549 |
| CPSF6 | ENY2 | 12 | 8 | A549 |
| CPSF6 | EZH2 | 12 | 7 | A549 |
| CPSF6 | HDAC7 | 12 | 12 | A549 |
| CPSF6 | HNRPDL | 12 | 4 | A549 |
| CPSF6 | NUP107 | 12 | 12 | A549 |
| CPSF6 | PDE4B | 12 | 1 | A549 |
| CPSF6 | RAP1B | 12 | 12 | A549 |
| CPSF6 | RPL3 | 12 | 22 | A549 |
| CPSF6 | SPG7 | 12 | 16 | A549 |
| CPSF6 | TAF1 | 12 | 11 | A549 |
| CYP24A1 | AKR1E2 | 20 | 10 | A549 |
| CYP24A1 | C9orf3 | 20 | 9 | A549 |
| CYP24A1 | CDK12 | 20 | 17 | A549 |
| CYP24A1 | CDK12 | 20 | 17 | A549 |
| CYP24A1 | CLDND2 | 20 | 19 | A549 |
| CYP24A1 | CYHR1 | 20 | 8 | A549 |
| CYP24A1 | CYHR1 | 20 | 8 | A549 |
| CYP24A1 | DAP3 | 20 | 1 | A549 |
| CYP24A1 | DDX5 | 20 | 17 | A549 |
| CYP24A1 | FNIP1 | 20 | 5 | A549 |
| CYP24A1 | HEATR2 | 20 | 7 | A549 |
| CYP24A1 | KAT5 | 20 | 11 | A549 |
| CYP24A1 | LAPTM4B | 20 | 8 | A549 |
| CYP24A1 | LEMD2 | 20 | 6 | A549 |
| CYP24A1 | LMNB2 | 20 | 19 | A549 |
| CYP24A1 | LOC100049716 | 20 | 12 | A549 |
| CYP24A1 | LRP5 | 20 | 11 | A549 |
| CYP24A1 | OTUD3 | 20 | 1 | A549 |
| CYP24A1 | PRKCE | 20 | 2 | A549 |
| CYP24A1 | PRR13 | 20 | 12 | A549 |
| CYP24A1 | PRR13 | 20 | 12 | A549 |
| CYP24A1 | PSMC4 | 20 | 19 | A549 |
| CYP24A1 | SHARPIN | 20 | 8 | A549 |
| CYP24A1 | SLC25A37 | 20 | 8 | A549 |
| CYP24A1 | SPG7 | 20 | 16 | A549 |
| CYP24A1 | SPG7 | 20 | 16 | A549 |
| CYP24A1 | SRSF1 | 20 | 17 | A549 |
| CYP24A1 | STT3A | 20 | 11 | A549 |
| CYP24A1 | TCFL5 | 20 | 20 | A549 |
| CYP24A1 | TRIO | 20 | 5 | A549 |
| CYP24A1 | WDR4 | 20 | 21 | A549 |
| CYP24A1 | WWP2 | 20 | 16 | A549 |
| EEF1D | ARID1A | 8 | 1 | A549 |
| EEF1D | C19orf22 | 8 | 19 | A549 |
| EEF1D | C8orf55 | 8 | 8 | A549 |
| EEF1D | C9orf3 | 8 | 9 | A549 |
| EEF1D | CFL1 | 8 | 11 | A549 |
| EEF1D | GTF3C2 | 8 | 2 | A549 |
| EEF1D | HDAC4 | 8 | 2 | A549 |
| EEF1D | LZTS2 | 8 | 10 | A549 |
| EEF1D | MCOLN1 | 8 | 19 | A549 |
| EEF1D | NME4 | 8 | 16 | A549 |
| EEF1D | TSPAN4 | 8 | 11 | A549 |
| EEF1D | TSSC1 | 8 | 2 | A549 |
| EEF1D | ZC3H3 | 8 | 8 | A549 |
| EEF1D | ZC3H3 | 8 | 8 | A549 |
| EEF1D | ZC3H3 | 8 | 8 | A549 |
| EEF1D | ZNF598 | 8 | 16 | A549 |
| MAD1L1 | CARKD | 7 | 13 | A549 |
| MAD1L1 | EIF3B | 7 | 7 | A549 |
| MAD1L1 | FAM20C | 7 | 7 | A549 |
| MAD1L1 | MAFK | 7 | 7 | A549 |
| MAD1L1 | MAFK | 7 | 7 | A549 |
| MAD1L1 | MAFK | 7 | 7 | A549 |
| ncRNA00188 | ALDH1A1 | 17 | 9 | A549 |
| ncRNA00188 | CKAP5 | 17 | 11 | A549 |
| ncRNA00188 | OVOL2 | 17 | 20 | A549 |
| ncRNA00188 | PCSK5 | 17 | 9 | A549 |
| ncRNA00188 | UBB | 17 | 17 | A549 |
| ncRNA00188 | WHSC1 | 17 | 4 | A549 |
| PLEC | ANKLE2 | 8 | 12 | A549 |
| PLEC | EEF1D | 8 | 8 | A549 |
| PLEC | EEF1D | 8 | 8 | A549 |
| PLEC | HEATR7A | 8 | 8 | A549 |
| PLEC | HEATR7A | 8 | 8 | A549 |
| PLEC | KLHDC2 | 8 | 14 | A549 |
| PLEC | NAT8L | 8 | 4 | A549 |
| PLEC | NUDT14 | 8 | 14 | A549 |
| PLEC | RNF126 | 8 | 19 | A549 |
| PLEC | SDC1 | 8 | 2 | A549 |
| PLEC | SHARPIN | 8 | 8 | A549 |
| PLEC | SHARPIN | 8 | 8 | A549 |
| PLEC | TPP1 | 8 | 11 | A549 |
| PPP1R12C | ALDOA | 19 | 16 | A549 |
| PPP1R12C | ASPSCR1 | 19 | 17 | A549 |
| PPP1R12C | CNN2 | 19 | 19 | A549 |
| PPP1R12C | FOSL1 | 19 | 11 | A549 |
| PPP1R12C | TSPAN4 | 19 | 11 | A549 |
| SNHG3 | ATP6V1G2 | 1 | 6 | A549 |
| SNHG3 | CUL3 | 1 | 2 | A549 |
| SNHG3 | DHRS3 | 1 | 1 | A549 |
| SNHG3 | FEN1 | 1 | 11 | A549 |
| SNHG3 | FLNB | 1 | 3 | A549 |
| SNHG3 | HSP90AA1 | 1 | 14 | A549 |
| SNHG3 | 7-Mar | 1 | 2 | A549 |
| SNHG3 | NUP107 | 1 | 12 | A549 |
| SNHG3 | PHACTR4 | 1 | 1 | A549 |
| SNHG3 | PHACTR4 | 1 | 1 | A549 |
| SNHG3 | PTCD3 | 1 | 2 | A549 |
| SNHG3 | SHPK | 1 | 17 | A549 |
| SNHG3 | STK3 | 1 | 8 | A549 |
| SNHG3 | TRNAU1AP | 1 | 1 | A549 |
| SNHG3 | XPO1 | 1 | 2 | A549 |
| SNHG3 | ZNF638 | 1 | 2 | A549 |
| SNHG3 | ABCE1 | 1 | 4 | CUTLL |
| SNHG3 | CTCF | 1 | 16 | CUTLL |
| SNHG3 | GIGYF2 | 1 | 2 | CUTLL |
| SNHG3 | NFS1 | 1 | 20 | CUTLL |
| SNHG3 | PDXDC1 | 1 | 16 | CUTLL |
| SNHG3 | PKM2 | 1 | 15 | CUTLL |
| SNHG3 | POLE2 | 1 | 14 | CUTLL |
| SNHG3 | AKR1A1 | 1 | 1 | H460 |
| SNHG3 | DAP3 | 1 | 1 | H460 |
| SNHG3 | FDPS | 1 | 1 | H460 |
| SNHG3 | PRR13 | 1 | 12 | H460 |
| SNHG3 | PSMD3 | 1 | 17 | H460 |
| SNHG3 | RPF2 | 1 | 6 | H460 |
| SNHG3 | RRP36 | 1 | 6 | H460 |
| SNHG3 | SETX | 1 | 9 | H460 |
| SNHG3 | SMARCAD1 | 1 | 4 | H460 |
| SNHG3 | VASP | 1 | 19 | H460 |
| SNHG3 | CCT3 | 1 | 1 | HCT116 |
| SNHG3 | CSNK1A1 | 1 | 5 | HCT116 |
| SNHG3 | GNB1 | 1 | 1 | HCT116 |
| SNHG3 | HAUS1 | 1 | 18 | HCT116 |
| SNHG3 | HSPE1 | 1 | 2 | HCT116 |
| SNHG3 | MIIP | 1 | 1 | HCT116 |
| SNHG3 | NFYB | 1 | 12 | HCT116 |
| SNHG3 | PDXDC1 | 1 | 16 | HCT116 |
| SNHG3 | PSMG3 | 1 | 7 | HCT116 |
| SNHG3 | RPLP0 | 1 | 12 | HCT116 |
| SNHG3 | SERINC2 | 1 | 1 | HCT116 |
| SNHG3 | TRNAU1AP | 1 | 1 | HCT116 |
| SNHG3 | ANXA2 | 1 | 15 | Hela-3 |
| SNHG3 | DDX17 | 1 | 22 | Hela-3 |
| SNHG3 | ENO1 | 1 | 1 | Hela-3 |
| SNHG3 | FAF1 | 1 | 1 | Hela-3 |
| SNHG3 | FIP1L1 | 1 | 4 | Hela-3 |
| SNHG3 | GDI2 | 1 | 10 | Hela-3 |
| SNHG3 | GIGYF2 | 1 | 2 | Hela-3 |
| SNHG3 | GNAS | 1 | 20 | Hela-3 |
| SNHG3 | INCENP | 1 | 11 | Hela-3 |
| SNHG3 | ITGB3BP | 1 | 1 | Hela-3 |
| SNHG3 | NDUFS1 | 1 | 2 | Hela-3 |
| SNHG3 | PFKP | 1 | 10 | Hela-3 |
| SNHG3 | PKM2 | 1 | 15 | Hela-3 |
| SNHG3 | PRMT5 | 1 | 14 | Hela-3 |
| SNHG3 | RFWD2 | 1 | 1 | Hela-3 |
| SNHG3 | SENP3 | 1 | 17 | Hela-3 |
| SNHG3 | SNHG12 | 1 | 1 | Hela-3 |
| SNHG3 | TRNAU1AP | 1 | 1 | Hela-3 |
| SNHG3 | UBR5 | 1 | 8 | Hela-3 |
| SNHG4 | ANKLE2 | 5 | 12 | Hela-3 |
| SNHG4 | CLCN7 | 5 | 16 | Hela-3 |
| SNHG4 | KIAA0368 | 5 | 9 | Hela-3 |
| SNHG4 | MBD2 | 5 | 18 | Hela-3 |
| SNHG4 | UBE2D2 | 5 | 5 | Hela-3 |
| EEF1D | GNAS | 8 | 20 | HepG2 |
| EEF1D | MAD1L1 | 8 | 7 | HepG2 |
| EEF1D | PTPRN2 | 8 | 7 | HepG2 |
| EEF1D | SHC2 | 8 | 19 | HepG2 |
| EEF1D | TSPAN4 | 8 | 11 | HepG2 |
| EEF1D | TSTA3 | 8 | 8 | HepG2 |
| ELL2 | CAST | 5 | 5 | HepG2 |
| ELL2 | CAST | 5 | 5 | HepG2 |
| ELL2 | PFDN1 | 5 | 5 | HepG2 |
| ELL2 | PFDN1 | 5 | 5 | HepG2 |
| ELL2 | RHOBTB3 | 5 | 5 | HepG2 |
| HECTD1 | AFP | 14 | 4 | HepG2 |
| HECTD1 | ARHGAP5 | 14 | 14 | HepG2 |
| HECTD1 | C14orf126 | 14 | 14 | HepG2 |
| HECTD1 | C14orf126 | 14 | 14 | HepG2 |
| HECTD1 | PVRL3 | 14 | 3 | HepG2 |
| HECTD1 | STRN3 | 14 | 14 | HepG2 |
| HNRNPH1 | CTTN | 5 | 11 | HepG2 |
| HNRNPH1 | DDB1 | 5 | 11 | HepG2 |
| HNRNPH1 | FN1 | 5 | 2 | HepG2 |
| HNRNPH1 | GNAS | 5 | 20 | HepG2 |
| HNRNPH1 | IGF1R | 5 | 15 | HepG2 |
| HNRNPH1 | SQSTM1 | 5 | 5 | HepG2 |
| LOC375010 | C14orf126 | 1 | 14 | HepG2 |
| LOC375010 | C14orf126 | 1 | 14 | HepG2 |
| LOC375010 | CSE1L | 1 | 20 | HepG2 |
| LOC375010 | CSE1L | 1 | 20 | HepG2 |
| LOC375010 | EEF1E1 | 1 | 6 | HepG2 |
| LOC375010 | EEF1E1 | 1 | 6 | HepG2 |
| LOC375010 | GOLGA8B | 1 | 15 | HepG2 |
| LOC375010 | HNRNPC | 1 | 14 | HepG2 |
| LOC375010 | KIAA0146 | 1 | 8 | HepG2 |
| LOC375010 | KIAA0146 | 1 | 8 | HepG2 |
| LOC375010 | PIK3C3 | 1 | 18 | HepG2 |
| LOC375010 | SEC23A | 1 | 14 | HepG2 |
| LOC375010 | SP140L | 1 | 2 | HepG2 |
| LOC375010 | ZFR | 1 | 5 | HepG2 |
| ncRNA00188 | ANXA2 | 17 | 15 | HepG2 |
| ncRNA00188 | ATR | 17 | 3 | HepG2 |
| ncRNA00188 | C19orf48 | 17 | 19 | HepG2 |
| ncRNA00188 | CTNNBL1 | 17 | 20 | HepG2 |
| ncRNA00188 | MRPL3 | 17 | 3 | HepG2 |
| ncRNA00188 | SND1 | 17 | 7 | HepG2 |
| ncRNA00188 | SNHG7 | 17 | 9 | HepG2 |
| ncRNA00188 | TPI1 | 17 | 12 | HepG2 |
| ncRNA00188 | UBAP2 | 17 | 9 | HepG2 |
| ncRNA00188 | WIPF2 | 17 | 17 | HepG2 |
| SNHG3 | AHSG | 1 | 3 | HepG2 |
| SNHG3 | AHSG | 1 | 3 | HepG2 |
| SNHG3 | ANKRD17 | 1 | 4 | HepG2 |
| SNHG3 | ATG9A | 1 | 2 | HepG2 |
| SNHG3 | ATP5B | 1 | 12 | HepG2 |
| SNHG3 | CCNT1 | 1 | 12 | HepG2 |
| SNHG3 | CDHR2 | 1 | 5 | HepG2 |
| SNHG3 | CSE1L | 1 | 20 | HepG2 |
| SNHG3 | DHRS3 | 1 | 1 | HepG2 |
| SNHG3 | DYNC1H1 | 1 | 14 | HepG2 |
| SNHG3 | EEF1D | 1 | 8 | HepG2 |
| SNHG3 | EIF3E | 1 | 8 | HepG2 |
| SNHG3 | ENO1 | 1 | 1 | HepG2 |
| SNHG3 | FARP1 | 1 | 13 | HepG2 |
| SNHG3 | FN1 | 1 | 2 | HepG2 |
| SNHG3 | FN1 | 1 | 2 | HepG2 |
| SNHG3 | GFPT1 | 1 | 2 | HepG2 |
| SNHG3 | GTF2IRD1 | 1 | 7 | HepG2 |
| SNHG3 | HAUS1 | 1 | 18 | HepG2 |
| SNHG3 | HNRNPC | 1 | 14 | HepG2 |
| SNHG3 | IMP3 | 1 | 15 | HepG2 |
| SNHG3 | KIF1B | 1 | 1 | HepG2 |
| SNHG3 | KIF2A | 1 | 5 | HepG2 |
| SNHG3 | LDHA | 1 | 11 | HepG2 |
| SNHG3 | LSM2 | 1 | 6 | HepG2 |
| SNHG3 | NFS1 | 1 | 20 | HepG2 |
| SNHG3 | NPL | 1 | 1 | HepG2 |
| SNHG3 | PPA1 | 1 | 10 | HepG2 |
| SNHG3 | PRR13 | 1 | 12 | HepG2 |
| SNHG3 | PSMB2 | 1 | 1 | HepG2 |
| SNHG3 | PSMD3 | 1 | 17 | HepG2 |
| SNHG3 | PUS7 | 1 | 7 | HepG2 |
| SNHG3 | RBM39 | 1 | 20 | HepG2 |
| SNHG3 | RPL17 | 1 | 18 | HepG2 |
| SNHG3 | RPL18A | 1 | 19 | HepG2 |
| SNHG3 | SEC24B | 1 | 4 | HepG2 |
| SNHG3 | SENP3 | 1 | 17 | HepG2 |
| SNHG3 | SNRPN | 1 | 15 | HepG2 |
| SNHG3 | SPNS1 | 1 | 16 | HepG2 |
| SNHG3 | SRSF1 | 1 | 17 | HepG2 |
| SNHG3 | SUN1 | 1 | 7 | HepG2 |
| SNHG3 | TAF12 | 1 | 1 | HepG2 |
| SNHG3 | TAF12 | 1 | 1 | HepG2 |
| SNHG3 | TBCA | 1 | 5 | HepG2 |
| SNHG3 | TCF25 | 1 | 16 | HepG2 |
| SNHG3 | TLK1 | 1 | 2 | HepG2 |
| SNHG3 | TRNAU1AP | 1 | 1 | HepG2 |
| SNHG3 | TRNP1 | 1 | 1 | HepG2 |
| SNHG3 | UIMC1 | 1 | 5 | HepG2 |
| SNHG3 | USP48 | 1 | 1 | HepG2 |
| SNHG3 | ZFYVE16 | 1 | 5 | HepG2 |
| SNHG4 | CTNNA1 | 5 | 5 | HepG2 |
| SNHG4 | ETF1 | 5 | 5 | HepG2 |
| SNHG4 | GTF2I | 5 | 7 | HepG2 |
| SNHG4 | HP1BP3 | 5 | 1 | HepG2 |
| SNHG4 | PAIP2 | 5 | 5 | HepG2 |
| SNHG4 | RHOA | 5 | 3 | HepG2 |
| SNHG4 | ROCK2 | 5 | 2 | HepG2 |
| SNHG4 | SIL1 | 5 | 5 | HepG2 |
| SNHG4 | EIF4G3 | 5 | 1 | HT1080 |
| SNHG4 | GLYR1 | 5 | 16 | HT1080 |
| SNHG4 | NVL | 5 | 1 | HT1080 |
| SNHG4 | PHF14 | 5 | 7 | HT1080 |
| SNHG4 | RTN3 | 5 | 11 | HT1080 |
| SNHG4 | UCHL5 | 5 | 1 | HT1080 |
| ACADM | AP1G1 | 1 | 16 | K562 |
| ACADM | AP1G1 | 1 | 16 | K562 |
| ACADM | C6orf191 | 1 | 6 | K562 |
| ACADM | MSH4 | 1 | 1 | K562 |
| ACADM | NPL | 1 | 1 | K562 |
| ACADM | NPL | 1 | 1 | K562 |
| ACADM | VCL | 1 | 10 | K562 |
| ACADM | VCL | 1 | 10 | K562 |
| C7orf44 | BLVRA | 7 | 7 | K562 |
| C7orf44 | PSMA2 | 7 | 7 | K562 |
| C7orf44 | PSMA2 | 7 | 7 | K562 |
| C7orf44 | TAX1BP1 | 7 | 7 | K562 |
| C7orf44 | TAX1BP1 | 7 | 7 | K562 |
| C7orf44 | URGCP | 7 | 7 | K562 |
| C7orf44 | WIPI2 | 7 | 7 | K562 |
| C7orf58 | GOSR2 | 7 | 17 | K562 |
| C7orf58 | NMU | 7 | 4 | K562 |
| C7orf58 | RPL13 | 7 | 16 | K562 |
| C7orf58 | TUBGCP6 | 7 | 22 | K562 |
| C7orf58 | UBAP2L | 7 | 1 | K562 |
| SNHG3 | RPL17 | 1 | 18 | HepG2 |
| SNHG3 | RPL18A | 1 | 19 | HepG2 |
| SNHG3 | SEC24B | 1 | 4 | HepG2 |
| SNHG3 | SENP3 | 1 | 17 | HepG2 |
| SNHG3 | SNRPN | 1 | 15 | HepG2 |
| SNHG3 | SPNS1 | 1 | 16 | HepG2 |
| SNHG3 | SRSF1 | 1 | 17 | HepG2 |
| SNHG3 | SUN1 | 1 | 7 | HepG2 |
| SNHG3 | TAF12 | 1 | 1 | HepG2 |
| SNHG3 | TAF12 | 1 | 1 | HepG2 |
| SNHG3 | TBCA | 1 | 5 | HepG2 |
| SNHG3 | TCF25 | 1 | 16 | HepG2 |
| SNHG3 | TLK1 | 1 | 2 | HepG2 |
| SNHG3 | TRNAU1AP | 1 | 1 | HepG2 |
| SNHG3 | TRNP1 | 1 | 1 | HepG2 |
| SNHG3 | UIMC1 | 1 | 5 | HepG2 |
| SNHG3 | USP48 | 1 | 1 | HepG2 |
| SNHG3 | ZFYVE16 | 1 | 5 | HepG2 |
| SNHG4 | CTNNA1 | 5 | 5 | HepG2 |
| SNHG4 | ETF1 | 5 | 5 | HepG2 |
| SNHG4 | GTF2I | 5 | 7 | HepG2 |
| SNHG4 | HP1BP3 | 5 | 1 | HepG2 |
| SNHG4 | PAIP2 | 5 | 5 | HepG2 |
| SNHG4 | RHOA | 5 | 3 | HepG2 |
| SNHG4 | ROCK2 | 5 | 2 | HepG2 |
| SNHG4 | SIL1 | 5 | 5 | HepG2 |
| SNHG4 | EIF4G3 | 5 | 1 | HT1080 |
| SNHG4 | GLYR1 | 5 | 16 | HT1080 |
| SNHG4 | NVL | 5 | 1 | HT1080 |
| SNHG4 | PHF14 | 5 | 7 | HT1080 |
| SNHG4 | RTN3 | 5 | 11 | HT1080 |
| SNHG4 | UCHL5 | 5 | 1 | HT1080 |
| ACADM | AP1G1 | 1 | 16 | K562 |
| ACADM | AP1G1 | 1 | 16 | K562 |
| ACADM | C6orf191 | 1 | 6 | K562 |
| ACADM | MSH4 | 1 | 1 | K562 |
| ACADM | NPL | 1 | 1 | K562 |
| ACADM | NPL | 1 | 1 | K562 |
| ACADM | VCL | 1 | 10 | K562 |
| ACADM | VCL | 1 | 10 | K562 |
| C7orf44 | BLVRA | 7 | 7 | K562 |
| C7orf44 | PSMA2 | 7 | 7 | K562 |
| C7orf44 | PSMA2 | 7 | 7 | K562 |
| C7orf44 | TAX1BP1 | 7 | 7 | K562 |
| C7orf44 | TAX1BP1 | 7 | 7 | K562 |
| C7orf44 | URGCP | 7 | 7 | K562 |
| C7orf44 | WIPI2 | 7 | 7 | K562 |
| C7orf58 | GOSR2 | 7 | 17 | K562 |
| C7orf58 | NMU | 7 | 4 | K562 |
| C7orf58 | RPL13 | 7 | 16 | K562 |
| C7orf58 | TUBGCP6 | 7 | 22 | K562 |
| C7orf58 | UBAP2L | 7 | 1 | K562 |
| CCDC26 | ASAP1 | 8 | 8 | K562 |
| CCDC26 | ASAP1 | 8 | 8 | K562 |
| CCDC26 | ASAP1 | 8 | 8 | K562 |
| CCDC26 | FAM49B | 8 | 8 | K562 |
| CCDC26 | FAM49B | 8 | 8 | K562 |
| CCDC26 | FAM49B | 8 | 8 | K562 |
| CCDC26 | LOC728724 | 8 | 8 | K562 |
| CCDC26 | LOC728724 | 8 | 8 | K562 |
| CCDC26 | LOC728724 | 8 | 8 | K562 |
| CCDC26 | LOC728724 | 8 | 8 | K562 |
| CCDC26 | PVT1 | 8 | 8 | K562 |
| CHD2 | CCNF | 15 | 16 | K562 |
| CHD2 | PCF11 | 15 | 11 | K562 |
| CHD2 | SDF2 | 15 | 17 | K562 |
| CHD2 | SEPSECS | 15 | 4 | K562 |
| CHD2 | SRSF1 | 15 | 17 | K562 |
| CPSF6 | BTK | 12 | 23 | K562 |
| CPSF6 | C6orf203 | 12 | 6 | K562 |
| CPSF6 | CCT2 | 12 | 12 | K562 |
| CPSF6 | CSNK1D | 12 | 17 | K562 |
| CPSF6 | FAM120AOS | 12 | 9 | K562 |
| CPSF6 | GCFC1 | 12 | 21 | K562 |
| CPSF6 | KIAA0586 | 12 | 14 | K562 |
| CPSF6 | MRPL44 | 12 | 2 | K562 |
| CPSF6 | UBE2L3 | 12 | 22 | K562 |
| CPSF6 | UQCRB | 12 | 8 | K562 |
| CTBP2 | ATE1 | 10 | 10 | K562 |
| CTBP2 | MAEA | 10 | 4 | K562 |
| CTBP2 | MAP4 | 10 | 3 | K562 |
| CTBP2 | METTL10 | 10 | 10 | K562 |
| CTBP2 | OCIAD1 | 10 | 4 | K562 |
| CTBP2 | PSMB1 | 10 | 6 | K562 |
| CTBP2 | ZRANB1 | 10 | 10 | K562 |
| EXOC4 | CHCHD3 | 7 | 7 | K562 |
| EXOC4 | CHCHD3 | 7 | 7 | K562 |
| EXOC4 | CHCHD3 | 7 | 7 | K562 |
| EXOC4 | CHCHD3 | 7 | 7 | K562 |
| EXOC4 | SHFM1 | 7 | 7 | K562 |
| EXOC4 | TMEM209 | 7 | 7 | K562 |
| EXOC4 | TMEM209 | 7 | 7 | K562 |
| EXOC4 | UBN2 | 7 | 7 | K562 |
| HDLBP | ANKMY1 | 2 | 2 | K562 |
| HDLBP | ANKMY1 | 2 | 2 | K562 |
| HDLBP | GNAS | 2 | 20 | K562 |
| HDLBP | NDUFA10 | 2 | 2 | K562 |
| HDLBP | PASK | 2 | 2 | K562 |
| HDLBP | THAP4 | 2 | 2 | K562 |
| HDLBP | TRPC4AP | 2 | 20 | K562 |
| HNRNPH1 | CANX | 5 | 5 | K562 |
| HNRNPH1 | IARS | 5 | 9 | K562 |
| HNRNPH1 | MAML1 | 5 | 5 | K562 |
| HNRNPH1 | NDC80 | 5 | 18 | K562 |
| HNRNPH1 | PPIG | 5 | 2 | K562 |
| HNRNPH1 | SQSTM1 | 5 | 5 | K562 |
| HNRNPH1 | TBCD | 5 | 17 | K562 |
| HNRNPH1 | TXN | 5 | 9 | K562 |
| KIAA0114 | FAF1 | 4 | 1 | K562 |
| KIAA0114 | FKBP4 | 4 | 12 | K562 |
| KIAA0114 | GNB1 | 4 | 1 | K562 |
| KIAA0114 | GNB1 | 4 | 1 | K562 |
| KIAA0114 | MIB1 | 4 | 18 | K562 |
| KIAA0114 | NRD1 | 4 | 1 | K562 |
| KIAA0114 | NRD1 | 4 | 1 | K562 |
| KIAA0114 | PHF14 | 4 | 7 | K562 |
| KIAA0114 | PHKB | 4 | 16 | K562 |
| KIAA0114 | PHKB | 4 | 16 | K562 |
| KIAA0114 | PICALM | 4 | 11 | K562 |
| KIAA0114 | PRKCB | 4 | 16 | K562 |
| KIAA0114 | PRKCB | 4 | 16 | K562 |
| KIAA0114 | RPL10 | 4 | 23 | K562 |
| KIAA0114 | RPL3 | 4 | 22 | K562 |
| KIAA0114 | TRAPPC3 | 4 | 1 | K562 |
| LOC728323 | FAM138E | 2 | 15 | K562 |
| LOC728323 | FLJ45340 | 2 | 7 | K562 |
| LOC728323 | RPL23AP53 | 2 | 8 | K562 |
| LOC728323 | RPL23AP53 | 2 | 8 | K562 |
| LOC728323 | RPL23AP79 | 2 | 19 | K562 |
| LOC728323 | WASH3P | 2 | 15 | K562 |
| MCM3APAS | C21orf56 | 21 | 21 | K562 |
| MCM3APAS | C21orf56 | 21 | 21 | K562 |
| MCM3APAS | DEPDC1B | 21 | 5 | K562 |
| MCM3APAS | PRPF40A | 21 | 2 | K562 |
| MCM3APAS | PTTG1 | 21 | 5 | K562 |
| MIR17HG | FAF1 | 13 | 1 | K562 |
| MIR17HG | NUP214 | 13 | 9 | K562 |
| MIR17HG | NUP214 | 13 | 9 | K562 |
| MIR17HG | PANK2 | 13 | 20 | K562 |
| MIR17HG | PAPD4 | 13 | 5 | K562 |
| ncRNA00188 | ANXA2 | 17 | 15 | K562 |
| ncRNA00188 | BAZ1B | 17 | 7 | K562 |
| ncRNA00188 | CKAP5 | 17 | 11 | K562 |
| ncRNA00188 | CTNNBL1 | 17 | 20 | K562 |
| ncRNA00188 | EIF5 | 17 | 14 | K562 |
| ncRNA00188 | IMMP2L | 17 | 7 | K562 |
| ncRNA00188 | MAD1L1 | 17 | 7 | K562 |
| ncRNA00188 | MAD1L1 | 17 | 7 | K562 |
| ncRNA00188 | PAIP2 | 17 | 5 | K562 |
| ncRNA00188 | PHF14 | 17 | 7 | K562 |
| ncRNA00188 | PHF14 | 17 | 7 | K562 |
| ncRNA00188 | RPS3 | 17 | 11 | K562 |
| ncRNA00188 | RPS3 | 17 | 11 | K562 |
| ncRNA00188 | SENP3 | 17 | 17 | K562 |
| ncRNA00188 | SND1 | 17 | 7 | K562 |
| ncRNA00188 | SNHG7 | 17 | 9 | K562 |
| ncRNA00188 | UBAP2 | 17 | 9 | K562 |
| RPL27A | APLP2 | 11 | 11 | K562 |
| RPL27A | APLP2 | 11 | 11 | K562 |
| RPL27A | BAT2L2 | 11 | 1 | K562 |
| RPL27A | BAT2L2 | 11 | 1 | K562 |
| RPL27A | CCNT1 | 11 | 12 | K562 |
| RPL27A | DDIT4 | 11 | 10 | K562 |
| RPL27A | HDLBP | 11 | 2 | K562 |
| RPL27A | NVL | 11 | 1 | K562 |
| RPL27A | PLAA | 11 | 9 | K562 |
| RPL27A | RABGAP1L | 11 | 1 | K562 |
| RPL27A | RABGAP1L | 11 | 1 | K562 |
| RPL27A | RNF149 | 11 | 2 | K562 |
| RPL27A | RPL35 | 11 | 9 | K562 |
| RPL27A | RPS27A | 11 | 2 | K562 |
| RPL27A | RPS27A | 11 | 2 | K562 |
| RPL27A | RPS3 | 11 | 11 | K562 |
| RPL27A | SMC4 | 11 | 3 | K562 |
| RPL27A | SMC4 | 11 | 3 | K562 |
| RPL27A | SND1 | 11 | 7 | K562 |
| RPL27A | SND1 | 11 | 7 | K562 |
| RPL27A | SRSF2IP | 11 | 12 | K562 |
| RPL27A | SRSF2IP | 11 | 12 | K562 |
| RPL27A | UBE2D2 | 11 | 5 | K562 |
| SNHG3 | ABCE1 | 1 | 4 | K562 |
| SNHG3 | ABHD3 | 1 | 18 | K562 |
| SNHG3 | ABHD3 | 1 | 18 | K562 |
| SNHG3 | ADCK2 | 1 | 7 | K562 |
| SNHG3 | ADCK2 | 1 | 7 | K562 |
| SNHG3 | AKR1A1 | 1 | 1 | K562 |
| SNHG3 | ALG3 | 1 | 3 | K562 |
| SNHG3 | ALG3 | 1 | 3 | K562 |
| SNHG3 | ANKHD1 | 1 | 5 | K562 |
| SNHG3 | ANP32B | 1 | 9 | K562 |
| SNHG3 | ANXA2 | 1 | 15 | K562 |
| SNHG3 | ARL6IP1 | 1 | 16 | K562 |
| SNHG3 | ARL6IP1 | 1 | 16 | K562 |
| SNHG3 | ARL6IP1 | 1 | 16 | K562 |
| SNHG3 | ATP13A3 | 1 | 3 | K562 |
| SNHG3 | ATP13A3 | 1 | 3 | K562 |
| SNHG3 | ATP5A1 | 1 | 18 | K562 |
| SNHG3 | ATP5A1 | 1 | 18 | K562 |
| SNHG3 | ATP5B | 1 | 12 | K562 |
| SNHG3 | ATP5B | 1 | 12 | K562 |
| SNHG3 | ATP6V1G2 | 1 | 6 | K562 |
| SNHG3 | ATP6V1G2 | 1 | 6 | K562 |
| SNHG3 | ATP6V1G2 | 1 | 6 | K562 |
| SNHG3 | ATP6V1G2 | 1 | 6 | K562 |
| SNHG3 | ATP6V1G2 | 1 | 6 | K562 |
| SNHG3 | BAIAP2L1 | 1 | 7 | K562 |
| SNHG3 | BAIAP2L1 | 1 | 7 | K562 |
| SNHG3 | BLVRB | 1 | 19 | K562 |
| SNHG3 | BLVRB | 1 | 19 | K562 |
| SNHG3 | C11orf48 | 1 | 11 | K562 |
| SNHG3 | C11orf48 | 1 | 11 | K562 |
| SNHG3 | C2orf24 | 1 | 2 | K562 |
| SNHG3 | C9orf5 | 1 | 9 | K562 |
| SNHG3 | C9orf5 | 1 | 9 | K562 |
| SNHG3 | CANX | 1 | 5 | K562 |
| SNHG3 | CANX | 1 | 5 | K562 |
| SNHG3 | CANX | 1 | 5 | K562 |
| SNHG3 | CCAR1 | 1 | 10 | K562 |
| SNHG3 | CCAR1 | 1 | 10 | K562 |
| SNHG3 | CCDC132 | 1 | 7 | K562 |
| SNHG3 | CCDC132 | 1 | 7 | K562 |
| SNHG3 | CCDC18 | 1 | 1 | K562 |
| SNHG3 | CCNY | 1 | 10 | K562 |
| SNHG3 | CCT3 | 1 | 1 | K562 |
| SNHG3 | CCT3 | 1 | 1 | K562 |
| SNHG3 | CCT5 | 1 | 5 | K562 |
| SNHG3 | CCT5 | 1 | 5 | K562 |
| SNHG3 | CCT8 | 1 | 21 | K562 |
| SNHG3 | CCT8 | 1 | 21 | K562 |
| SNHG3 | CENPE | 1 | 4 | K562 |
| SNHG3 | CENPE | 1 | 4 | K562 |
| SNHG3 | CHAF1A | 1 | 19 | K562 |
| SNHG3 | CHCHD3 | 1 | 7 | K562 |
| SNHG3 | CHCHD3 | 1 | 7 | K562 |
| SNHG3 | CNOT1 | 1 | 16 | K562 |
| SNHG3 | CNOT1 | 1 | 16 | K562 |
| SNHG3 | CNOT10 | 1 | 3 | K562 |
| SNHG3 | CNOT10 | 1 | 3 | K562 |
| SNHG3 | COPA | 1 | 1 | K562 |
| SNHG3 | COPA | 1 | 1 | K562 |
| SNHG3 | COX5A | 1 | 15 | K562 |
| SNHG3 | COX5A | 1 | 15 | K562 |
| SNHG3 | COX5A | 1 | 15 | K562 |
| SNHG3 | COX5B | 1 | 2 | K562 |
| SNHG3 | CRAMP1L | 1 | 16 | K562 |
| SNHG3 | CRAMP1L | 1 | 16 | K562 |
| SNHG3 | CSE1L | 1 | 20 | K562 |
| SNHG3 | CSE1L | 1 | 20 | K562 |
| SNHG3 | CSE1L | 1 | 20 | K562 |
| SNHG3 | CTCF | 1 | 16 | K562 |
| SNHG3 | CUL2 | 1 | 10 | K562 |
| SNHG3 | CUL2 | 1 | 10 | K562 |
| SNHG3 | CUL3 | 1 | 2 | K562 |
| SNHG3 | CWF19L1 | 1 | 10 | K562 |
| SNHG3 | CWF19L1 | 1 | 10 | K562 |
| SNHG3 | CYHR1 | 1 | 8 | K562 |
| SNHG3 | DAP3 | 1 | 1 | K562 |
| SNHG3 | DAP3 | 1 | 1 | K562 |
| SNHG3 | DAP3 | 1 | 1 | K562 |
| SNHG3 | DAP3 | 1 | 1 | K562 |
| SNHG3 | DARS | 1 | 2 | K562 |
| SNHG3 | DARS | 1 | 2 | K562 |
| SNHG3 | DCAF6 | 1 | 1 | K562 |
| SNHG3 | DCAF6 | 1 | 1 | K562 |
| SNHG3 | DCAF6 | 1 | 1 | K562 |
| SNHG3 | DCI | 1 | 16 | K562 |
| SNHG3 | DDX17 | 1 | 22 | K562 |
| SNHG3 | DDX17 | 1 | 22 | K562 |
| SNHG3 | DHRS3 | 1 | 1 | K562 |
| SNHG3 | DHX29 | 1 | 5 | K562 |
| SNHG3 | DHX29 | 1 | 5 | K562 |
| SNHG3 | DIP2B | 1 | 12 | K562 |
| SNHG3 | DIP2B | 1 | 12 | K562 |
| SNHG3 | DKC1 | 1 | 23 | K562 |
| SNHG3 | DKC1 | 1 | 23 | K562 |
| SNHG3 | DKC1 | 1 | 23 | K562 |
| SNHG3 | DKFZP686I15217 | 1 | 6 | K562 |
| SNHG3 | DKFZP686I15217 | 1 | 6 | K562 |
| SNHG3 | DNAJC11 | 1 | 1 | K562 |
| SNHG3 | DNAJC7 | 1 | 17 | K562 |
| SNHG3 | DNAJC7 | 1 | 17 | K562 |
| SNHG3 | DYNC1H1 | 1 | 14 | K562 |
| SNHG3 | EEF1B2 | 1 | 2 | K562 |
| SNHG3 | EEF1D | 1 | 8 | K562 |
| SNHG3 | EEF1D | 1 | 8 | K562 |
| SNHG3 | EIF2B1 | 1 | 12 | K562 |
| SNHG3 | EIF2B1 | 1 | 12 | K562 |
| SNHG3 | EIF2B3 | 1 | 1 | K562 |
| SNHG3 | EIF2B3 | 1 | 1 | K562 |
| SNHG3 | EIF3E | 1 | 8 | K562 |
| SNHG3 | ELP2 | 1 | 18 | K562 |
| SNHG3 | ELP2 | 1 | 18 | K562 |
| SNHG3 | ENO1 | 1 | 1 | K562 |
| SNHG3 | ENO1 | 1 | 1 | K562 |
| SNHG3 | EPB41 | 1 | 1 | K562 |
| SNHG3 | EPB41 | 1 | 1 | K562 |
| SNHG3 | EPS15 | 1 | 1 | K562 |
| SNHG3 | ESCO1 | 1 | 18 | K562 |
| SNHG3 | ESYT2 | 1 | 7 | K562 |
| SNHG3 | EXOC6 | 1 | 10 | K562 |
| SNHG3 | EXOC6 | 1 | 10 | K562 |
| SNHG3 | EXOC6 | 1 | 10 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG3 | FAF1 | 1 | 1 | K562 |
| SNHG3 | FARSB | 1 | 2 | K562 |
| SNHG3 | FASTKD1 | 1 | 2 | K562 |
| SNHG3 | FASTKD1 | 1 | 2 | K562 |
| SNHG3 | FN3KRP | 1 | 17 | K562 |
| SNHG3 | FTSJD2 | 1 | 6 | K562 |
| SNHG3 | GABPB2 | 1 | 1 | K562 |
| SNHG3 | GCFC1 | 1 | 21 | K562 |
| SNHG3 | GCFC1 | 1 | 21 | K562 |
| SNHG3 | GDI2 | 1 | 10 | K562 |
| SNHG3 | GDI2 | 1 | 10 | K562 |
| SNHG3 | GGPS1 | 1 | 1 | K562 |
| SNHG3 | GGPS1 | 1 | 1 | K562 |
| SNHG3 | GNB1 | 1 | 1 | K562 |
| SNHG3 | GNB2L1 | 1 | 5 | K562 |
| SNHG3 | GNB2L1 | 1 | 5 | K562 |
| SNHG3 | GPR98 | 1 | 5 | K562 |
| SNHG3 | GPR98 | 1 | 5 | K562 |
| SNHG3 | GPR98 | 1 | 5 | K562 |
| SNHG3 | GSPT1 | 1 | 16 | K562 |
| SNHG3 | GTF2IRD1 | 1 | 7 | K562 |
| SNHG3 | GTF3C6 | 1 | 6 | K562 |
| SNHG3 | GTF3C6 | 1 | 6 | K562 |
| SNHG3 | GTPBP4 | 1 | 10 | K562 |
| SNHG3 | H2AFV | 1 | 7 | K562 |
| SNHG3 | H2AFV | 1 | 7 | K562 |
| SNHG3 | HBS1L | 1 | 6 | K562 |
| SNHG3 | HMGA1 | 1 | 6 | K562 |
| SNHG3 | HNRNPC | 1 | 14 | K562 |
| SNHG3 | HNRNPH1 | 1 | 5 | K562 |
| SNHG3 | HNRNPH1 | 1 | 5 | K562 |
| SNHG3 | HNRNPH3 | 1 | 10 | K562 |
| SNHG3 | HNRNPH3 | 1 | 10 | K562 |
| SNHG3 | HNRNPH3 | 1 | 10 | K562 |
| SNHG3 | HSP90AA1 | 1 | 14 | K562 |
| SNHG3 | HSPC157 | 1 | 1 | K562 |
| SNHG3 | HSPE1 | 1 | 2 | K562 |
| SNHG3 | HUWE1 | 1 | 23 | K562 |
| SNHG3 | HUWE1 | 1 | 23 | K562 |
| SNHG3 | ILF2 | 1 | 1 | K562 |
| SNHG3 | ILF2 | 1 | 1 | K562 |
| SNHG3 | ILF2 | 1 | 1 | K562 |
| SNHG3 | ILF3 | 1 | 19 | K562 |
| SNHG3 | IMP3 | 1 | 15 | K562 |
| SNHG3 | KARS | 1 | 16 | K562 |
| SNHG3 | KARS | 1 | 16 | K562 |
| SNHG3 | KIF1B | 1 | 1 | K562 |
| SNHG3 | KIF1B | 1 | 1 | K562 |
| SNHG3 | KIF2A | 1 | 5 | K562 |
| SNHG3 | KIF2A | 1 | 5 | K562 |
| SNHG3 | KLK1 | 1 | 19 | K562 |
| SNHG3 | KRT222 | 1 | 17 | K562 |
| SNHG3 | KRT222 | 1 | 17 | K562 |
| SNHG3 | LARP4 | 1 | 12 | K562 |
| SNHG3 | LARS | 1 | 5 | K562 |
| SNHG3 | LARS | 1 | 5 | K562 |
| SNHG3 | LCP1 | 1 | 13 | K562 |
| SNHG3 | LCP1 | 1 | 13 | K562 |
| SNHG3 | LOC440944 | 1 | 3 | K562 |
| SNHG3 | LOC440944 | 1 | 3 | K562 |
| SNHG3 | LOC641298 | 1 | 16 | K562 |
| SNHG3 | LOC641298 | 1 | 16 | K562 |
| SNHG3 | LRRC47 | 1 | 1 | K562 |
| SNHG3 | LSM2 | 1 | 6 | K562 |
| SNHG3 | LYN | 1 | 8 | K562 |
| SNHG3 | LYN | 1 | 8 | K562 |
| SNHG3 | MAPK1 | 1 | 22 | K562 |
| SNHG3 | MAPK1 | 1 | 22 | K562 |
| SNHG3 | MAPK1 | 1 | 22 | K562 |
| SNHG3 | MAPK1 | 1 | 22 | K562 |
| SNHG3 | MBD2 | 1 | 18 | K562 |
| SNHG3 | MBD2 | 1 | 18 | K562 |
| SNHG3 | MCM8 | 1 | 20 | K562 |
| SNHG3 | MDH2 | 1 | 7 | K562 |
| SNHG3 | METT10D | 1 | 17 | K562 |
| SNHG3 | METT10D | 1 | 17 | K562 |
| SNHG3 | MFF | 1 | 2 | K562 |
| SNHG3 | MRPL3 | 1 | 3 | K562 |
| SNHG3 | MTOR | 1 | 1 | K562 |
| SNHG3 | MYBL2 | 1 | 20 | K562 |
| SNHG3 | MYL6B | 1 | 12 | K562 |
| SNHG3 | MYL6B | 1 | 12 | K562 |
| SNHG3 | NDUFAF4 | 1 | 6 | K562 |
| SNHG3 | NNT | 1 | 5 | K562 |
| SNHG3 | NNT | 1 | 5 | K562 |
| SNHG3 | NNT | 1 | 5 | K562 |
| SNHG3 | NPL | 1 | 1 | K562 |
| SNHG3 | NPL | 1 | 1 | K562 |
| SNHG3 | NPL | 1 | 1 | K562 |
| SNHG3 | NPL | 1 | 1 | K562 |
| SNHG3 | NPM1 | 1 | 5 | K562 |
| SNHG3 | NPM1 | 1 | 5 | K562 |
| SNHG3 | NSMCE2 | 1 | 8 | K562 |
| SNHG3 | NSMCE2 | 1 | 8 | K562 |
| SNHG3 | NUDCD2 | 1 | 5 | K562 |
| SNHG3 | NUDCD2 | 1 | 5 | K562 |
| SNHG3 | NUP107 | 1 | 12 | K562 |
| SNHG3 | NUP214 | 1 | 9 | K562 |
| SNHG3 | NUP214 | 1 | 9 | K562 |
| SNHG3 | ODC1 | 1 | 2 | K562 |
| SNHG3 | ODC1 | 1 | 2 | K562 |
| SNHG3 | ODC1 | 1 | 2 | K562 |
| SNHG3 | OVOL2 | 1 | 20 | K562 |
| SNHG3 | OVOL2 | 1 | 20 | K562 |
| SNHG3 | PABPC4 | 1 | 1 | K562 |
| SNHG3 | PAK1IP1 | 1 | 6 | K562 |
| SNHG3 | PAK1IP1 | 1 | 6 | K562 |
| SNHG3 | PARK7 | 1 | 1 | K562 |
| SNHG3 | PARK7 | 1 | 1 | K562 |
| SNHG3 | PARP4 | 1 | 13 | K562 |
| SNHG3 | PARP4 | 1 | 13 | K562 |
| SNHG3 | PDCL2 | 1 | 4 | K562 |
| SNHG3 | PDS5A | 1 | 4 | K562 |
| SNHG3 | PFKP | 1 | 10 | K562 |
| SNHG3 | PHACTR4 | 1 | 1 | K562 |
| SNHG3 | PHACTR4 | 1 | 1 | K562 |
| SNHG3 | PHF14 | 1 | 7 | K562 |
| SNHG3 | PHF20 | 1 | 20 | K562 |
| SNHG3 | PHKB | 1 | 16 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PKM2 | 1 | 15 | K562 |
| SNHG3 | PKN1 | 1 | 19 | K562 |
| SNHG3 | PLEKHA4 | 1 | 19 | K562 |
| SNHG3 | PLEKHA4 | 1 | 19 | K562 |
| SNHG3 | POLE | 1 | 12 | K562 |
| SNHG3 | POLE | 1 | 12 | K562 |
| SNHG3 | POLE2 | 1 | 14 | K562 |
| SNHG3 | PPA1 | 1 | 10 | K562 |
| SNHG3 | PPM1B | 1 | 2 | K562 |
| SNHG3 | PPM1B | 1 | 2 | K562 |
| SNHG3 | PRAME | 1 | 22 | K562 |
| SNHG3 | PRAME | 1 | 22 | K562 |
| SNHG3 | PRAME | 1 | 22 | K562 |
| SNHG3 | PRKCB | 1 | 16 | K562 |
| SNHG3 | PRKCB | 1 | 16 | K562 |
| SNHG3 | PRKDC | 1 | 8 | K562 |
| SNHG3 | PRMT5 | 1 | 14 | K562 |
| SNHG3 | PRPF3 | 1 | 1 | K562 |
| SNHG3 | PRPF6 | 1 | 20 | K562 |
| SNHG3 | PRPF6 | 1 | 20 | K562 |
| SNHG3 | PRR13 | 1 | 12 | K562 |
| SNHG3 | PRR13 | 1 | 12 | K562 |
| SNHG3 | PSMA1 | 1 | 11 | K562 |
| SNHG3 | PSMA1 | 1 | 11 | K562 |
| SNHG3 | PSMD4 | 1 | 1 | K562 |
| SNHG3 | PSMD4 | 1 | 1 | K562 |
| SNHG3 | PSMD4 | 1 | 1 | K562 |
| SNHG3 | PSMG3 | 1 | 7 | K562 |
| SNHG3 | PSMG3 | 1 | 7 | K562 |
| SNHG3 | PTCD3 | 1 | 2 | K562 |
| SNHG3 | PUS7 | 1 | 7 | K562 |
| SNHG3 | PUS7 | 1 | 7 | K562 |
| SNHG3 | QRICH2 | 1 | 17 | K562 |
| SNHG3 | RANBP1 | 1 | 22 | K562 |
| SNHG3 | NSMCE2 | 1 | 8 | K562 |
| SNHG3 | NSMCE2 | 1 | 8 | K562 |
| SNHG3 | NUDCD2 | 1 | 5 | K562 |
| SNHG3 | NUDCD2 | 1 | 5 | K562 |
| SNHG3 | NUP107 | 1 | 12 | K562 |
| SNHG3 | NUP214 | 1 | 9 | K562 |
| SNHG3 | NUP214 | 1 | 9 | K562 |
| SNHG3 | ODC1 | 1 | 2 | K562 |
| SNHG3 | ODC1 | 1 | 2 | K562 |
| SNHG3 | ODC1 | 1 | 2 | K562 |
| SNHG3 | OVOL2 | 1 | 20 | K562 |
| SNHG3 | OVOL2 | 1 | 20 | K562 |
| SNHG3 | PABPC4 | 1 | 1 | K562 |
| SNHG3 | PAK1IP1 | 1 | 6 | K562 |
| SNHG3 | PAK1IP1 | 1 | 6 | K562 |
| SNHG3 | PARK7 | 1 | 1 | K562 |
| SNHG3 | PARK7 | 1 | 1 | K562 |
| SNHG3 | PARP4 | 1 | 13 | K562 |
| SNHG3 | PARP4 | 1 | 13 | K562 |
| SNHG3 | PDCL2 | 1 | 4 | K562 |
| SNHG3 | PDS5A | 1 | 4 | K562 |
| SNHG3 | PFKP | 1 | 10 | K562 |
| SNHG3 | PHACTR4 | 1 | 1 | K562 |
| SNHG3 | PHACTR4 | 1 | 1 | K562 |
| SNHG3 | PHF14 | 1 | 7 | K562 |
| SNHG3 | PHF20 | 1 | 20 | K562 |
| SNHG3 | PHKB | 1 | 16 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PICALM | 1 | 11 | K562 |
| SNHG3 | PKM2 | 1 | 15 | K562 |
| SNHG3 | PKN1 | 1 | 19 | K562 |
| SNHG3 | PLEKHA4 | 1 | 19 | K562 |
| SNHG3 | PLEKHA4 | 1 | 19 | K562 |
| SNHG3 | POLE | 1 | 12 | K562 |
| SNHG3 | POLE | 1 | 12 | K562 |
| SNHG3 | POLE2 | 1 | 14 | K562 |
| SNHG3 | PPA1 | 1 | 10 | K562 |
| SNHG3 | PPM1B | 1 | 2 | K562 |
| SNHG3 | PPM1B | 1 | 2 | K562 |
| SNHG3 | PRAME | 1 | 22 | K562 |
| SNHG3 | PRAME | 1 | 22 | K562 |
| SNHG3 | PRAME | 1 | 22 | K562 |
| SNHG3 | PRKCB | 1 | 16 | K562 |
| SNHG3 | PRKCB | 1 | 16 | K562 |
| SNHG3 | PRKDC | 1 | 8 | K562 |
| SNHG3 | PRMT5 | 1 | 14 | K562 |
| SNHG3 | PRPF3 | 1 | 1 | K562 |
| SNHG3 | PRPF6 | 1 | 20 | K562 |
| SNHG3 | PRPF6 | 1 | 20 | K562 |
| SNHG3 | PRR13 | 1 | 12 | K562 |
| SNHG3 | PRR13 | 1 | 12 | K562 |
| SNHG3 | PSMA1 | 1 | 11 | K562 |
| SNHG3 | PSMA1 | 1 | 11 | K562 |
| SNHG3 | PSMD4 | 1 | 1 | K562 |
| SNHG3 | PSMD4 | 1 | 1 | K562 |
| SNHG3 | PSMD4 | 1 | 1 | K562 |
| SNHG3 | PSMG3 | 1 | 7 | K562 |
| SNHG3 | PSMG3 | 1 | 7 | K562 |
| SNHG3 | PTCD3 | 1 | 2 | K562 |
| SNHG3 | PUS7 | 1 | 7 | K562 |
| SNHG3 | PUS7 | 1 | 7 | K562 |
| SNHG3 | QRICH2 | 1 | 17 | K562 |
| SNHG3 | RANBP1 | 1 | 22 | K562 |
| SNHG3 | RANBP1 | 1 | 22 | K562 |
| SNHG3 | RBM16 | 1 | 6 | K562 |
| SNHG3 | RBM16 | 1 | 6 | K562 |
| SNHG3 | RBM39 | 1 | 20 | K562 |
| SNHG3 | RBM39 | 1 | 20 | K562 |
| SNHG3 | RBM39 | 1 | 20 | K562 |
| SNHG3 | RFWD2 | 1 | 1 | K562 |
| SNHG3 | RHAG | 1 | 6 | K562 |
| SNHG3 | RHAG | 1 | 6 | K562 |
| SNHG3 | RHAG | 1 | 6 | K562 |
| SNHG3 | RHAG | 1 | 6 | K562 |
| SNHG3 | RHEB | 1 | 7 | K562 |
| SNHG3 | RHOA | 1 | 3 | K562 |
| SNHG3 | RNASEH1 | 1 | 2 | K562 |
| SNHG3 | RNF149 | 1 | 2 | K562 |
| SNHG3 | RNF149 | 1 | 2 | K562 |
| SNHG3 | RNF4 | 1 | 4 | K562 |
| SNHG3 | RNF4 | 1 | 4 | K562 |
| SNHG3 | RPL17 | 1 | 18 | K562 |
| SNHG3 | RPL18A | 1 | 19 | K562 |
| SNHG3 | RPL22 | 1 | 1 | K562 |
| SNHG3 | RPL23 | 1 | 17 | K562 |
| SNHG3 | RPL23 | 1 | 17 | K562 |
| SNHG3 | RPL23 | 1 | 17 | K562 |
| SNHG3 | RPL3 | 1 | 22 | K562 |
| SNHG3 | RPL3 | 1 | 22 | K562 |
| SNHG3 | RPL30 | 1 | 8 | K562 |
| SNHG3 | RPL30 | 1 | 8 | K562 |
| SNHG3 | RPL4 | 1 | 15 | K562 |
| SNHG3 | RPL4 | 1 | 15 | K562 |
| SNHG3 | RPL5 | 1 | 1 | K562 |
| SNHG3 | RPN2 | 1 | 20 | K562 |
| SNHG3 | RPN2 | 1 | 20 | K562 |
| SNHG3 | RPS18 | 1 | 6 | K562 |
| SNHG3 | RPS3 | 1 | 11 | K562 |
| SNHG3 | RPS3 | 1 | 11 | K562 |
| SNHG3 | RPS5 | 1 | 19 | K562 |
| SNHG3 | RPS5 | 1 | 19 | K562 |
| SNHG3 | RPS6KC1 | 1 | 1 | K562 |
| SNHG3 | SAGE1 | 1 | 23 | K562 |
| SNHG3 | SDHB | 1 | 1 | K562 |
| SNHG3 | SDHB | 1 | 1 | K562 |
| SNHG3 | SEC24B | 1 | 4 | K562 |
| SNHG3 | SEC24B | 1 | 4 | K562 |
| SNHG3 | SENP3 | 1 | 17 | K562 |
| SNHG3 | SENP3 | 1 | 17 | K562 |
| SNHG3 | SENP3 | 1 | 17 | K562 |
| SNHG3 | 2-Sep | 1 | 2 | K562 |
| SNHG3 | 2-Sep | 1 | 2 | K562 |
| SNHG3 | SERPINB6 | 1 | 6 | K562 |
| SNHG3 | SERPINB6 | 1 | 6 | K562 |
| SNHG3 | SETX | 1 | 9 | K562 |
| SNHG3 | SF3B3 | 1 | 16 | K562 |
| SNHG3 | SF3B3 | 1 | 16 | K562 |
| SNHG3 | SHPK | 1 | 17 | K562 |
| SNHG3 | SIKE1 | 1 | 1 | K562 |
| SNHG3 | SIKE1 | 1 | 1 | K562 |
| SNHG3 | SKI | 1 | 1 | K562 |
| SNHG3 | SLC38A10 | 1 | 17 | K562 |
| SNHG3 | SMARCAD1 | 1 | 4 | K562 |
| SNHG3 | SMARCC1 | 1 | 3 | K562 |
| SNHG3 | SMARCC1 | 1 | 3 | K562 |
| SNHG3 | SMC6 | 1 | 2 | K562 |
| SNHG3 | SMC6 | 1 | 2 | K562 |
| SNHG3 | SNHG12 | 1 | 1 | K562 |
| SNHG3 | SNHG12 | 1 | 1 | K562 |
| SNHG3 | SNORD1C | 1 | 17 | K562 |
| SNHG3 | SNRPD3 | 1 | 22 | K562 |
| SNHG3 | SNRPD3 | 1 | 22 | K562 |
| SNHG3 | SON | 1 | 21 | K562 |
| SNHG3 | SON | 1 | 21 | K562 |
| SNHG3 | SON | 1 | 21 | K562 |
| SNHG3 | SP1 | 1 | 12 | K562 |
| SNHG3 | SPTA1 | 1 | 1 | K562 |
| SNHG3 | SPTA1 | 1 | 1 | K562 |
| SNHG3 | SRP54 | 1 | 14 | K562 |
| SNHG3 | SRP54 | 1 | 14 | K562 |
| SNHG3 | SRP72 | 1 | 4 | K562 |
| SNHG3 | SRP72 | 1 | 4 | K562 |
| SNHG3 | SRSF1 | 1 | 17 | K562 |
| SNHG3 | SRSF11 | 1 | 1 | K562 |
| SNHG3 | SRSF11 | 1 | 1 | K562 |
| SNHG3 | STAG2 | 1 | 23 | K562 |
| SNHG3 | STAG2 | 1 | 23 | K562 |
| SNHG3 | STAT5B | 1 | 17 | K562 |
| SNHG3 | STAT5B | 1 | 17 | K562 |
| SNHG3 | STAT5B | 1 | 17 | K562 |
| SNHG3 | STIL | 1 | 1 | K562 |
| SNHG3 | STIL | 1 | 1 | K562 |
| SNHG3 | STIP1 | 1 | 11 | K562 |
| SNHG3 | STK3 | 1 | 8 | K562 |
| SNHG3 | STRBP | 1 | 9 | K562 |
| SNHG3 | STRBP | 1 | 9 | K562 |
| SNHG3 | STRBP | 1 | 9 | K562 |
| SNHG3 | TAF12 | 1 | 1 | K562 |
| SNHG3 | TAF12 | 1 | 1 | K562 |
| SNHG3 | TAF12 | 1 | 1 | K562 |
| SNHG3 | TBCA | 1 | 5 | K562 |
| SNHG3 | TBCA | 1 | 5 | K562 |
| SNHG3 | TCF25 | 1 | 16 | K562 |
| SNHG3 | TCP1 | 1 | 6 | K562 |
| SNHG3 | TCP1 | 1 | 6 | K562 |
| SNHG3 | TFPI | 1 | 2 | K562 |
| SNHG3 | TFPI | 1 | 2 | K562 |
| SNHG3 | TOPBP1 | 1 | 3 | K562 |
| SNHG3 | TOPBP1 | 1 | 3 | K562 |
| SNHG3 | TRAP1 | 1 | 16 | K562 |
| SNHG3 | TRAP1 | 1 | 16 | K562 |
| SNHG3 | TRIM33 | 1 | 1 | K562 |
| SNHG3 | TRIM33 | 1 | 1 | K562 |
| SNHG3 | TRNAU1AP | 1 | 1 | K562 |
| SNHG3 | TRNAU1AP | 1 | 1 | K562 |
| SNHG3 | TRNAU1AP | 1 | 1 | K562 |
| SNHG3 | TRNAU1AP | 1 | 1 | K562 |
| SNHG3 | TSR1 | 1 | 17 | K562 |
| SNHG3 | TTC17 | 1 | 11 | K562 |
| SNHG3 | TTC17 | 1 | 11 | K562 |
| SNHG3 | TYW1 | 1 | 7 | K562 |
| SNHG3 | U2AF1 | 1 | 21 | K562 |
| SNHG3 | U2AF1 | 1 | 21 | K562 |
| SNHG3 | UAP1 | 1 | 1 | K562 |
| SNHG3 | UAP1 | 1 | 1 | K562 |
| SNHG3 | UBAP2 | 1 | 9 | K562 |
| SNHG3 | UBAP2 | 1 | 9 | K562 |
| SNHG3 | UBAP2 | 1 | 9 | K562 |
| SNHG3 | UBAP2 | 1 | 9 | K562 |
| SNHG3 | UBB | 1 | 17 | K562 |
| SNHG3 | UBE2I | 1 | 16 | K562 |
| SNHG3 | UBE2I | 1 | 16 | K562 |
| SNHG3 | UBE3C | 1 | 7 | K562 |
| SNHG3 | UBE3C | 1 | 7 | K562 |
| SNHG3 | UBR5 | 1 | 8 | K562 |
| SNHG3 | UCHL5 | 1 | 1 | K562 |
| SNHG3 | UCHL5 | 1 | 1 | K562 |
| SNHG3 | UIMC1 | 1 | 5 | K562 |
| SNHG3 | USP48 | 1 | 1 | K562 |
| SNHG3 | UTP6 | 1 | 17 | K562 |
| SNHG3 | WDHD1 | 1 | 14 | K562 |
| SNHG3 | WDHD1 | 1 | 14 | K562 |
| SNHG3 | WDR43 | 1 | 2 | K562 |
| SNHG3 | WDR43 | 1 | 2 | K562 |
| SNHG3 | WHSC1 | 1 | 4 | K562 |
| SNHG3 | WHSC1 | 1 | 4 | K562 |
| SNHG3 | XPO1 | 1 | 2 | K562 |
| SNHG3 | YLPM1 | 1 | 14 | K562 |
| SNHG3 | YLPM1 | 1 | 14 | K562 |
| SNHG3 | YY1AP1 | 1 | 1 | K562 |
| SNHG3 | ZBED5 | 1 | 11 | K562 |
| SNHG3 | ZBTB8OS | 1 | 1 | K562 |
| SNHG3 | ZBTB8OS | 1 | 1 | K562 |
| SNHG3 | ZCCHC7 | 1 | 9 | K562 |
| SNHG3 | ZCCHC7 | 1 | 9 | K562 |
| SNHG3 | ZCCHC7 | 1 | 9 | K562 |
| SNHG3 | ZFR | 1 | 5 | K562 |
| SNHG3 | ZNF431 | 1 | 19 | K562 |
| SNHG3 | ZNF431 | 1 | 19 | K562 |
| SNHG3 | ZNF638 | 1 | 2 | K562 |
| SNHG3 | ZNF713 | 1 | 7 | K562 |
| SNHG3 | ZNF713 | 1 | 7 | K562 |
| SNHG3 | ZNF713 | 1 | 7 | K562 |
| SNHG4 | AGPS | 5 | 2 | K562 |
| SNHG4 | AGPS | 5 | 2 | K562 |
| SNHG4 | ATXN2 | 5 | 12 | K562 |
| SNHG4 | GNAS | 5 | 20 | K562 |
| SNHG4 | GTF2I | 5 | 7 | K562 |
| SNHG4 | NS3BP | 5 | 11 | K562 |
| SNHG4 | NS3BP | 5 | 11 | K562 |
| SNHG4 | PICALM | 5 | 11 | K562 |
| SNHG4 | PICALM | 5 | 11 | K562 |
| SNHG4 | PSMD1 | 5 | 2 | K562 |
| SNHG4 | RPS27A | 5 | 2 | K562 |
| SNHG4 | RPS27A | 5 | 2 | K562 |
| SNHG4 | RRN3P3 | 5 | 16 | K562 |
| SNHG4 | SKP1 | 5 | 5 | K562 |
| SNHG4 | TMEM66 | 5 | 8 | K562 |
| SNHG4 | UBE2K | 5 | 4 | K562 |
| SNHG4 | UBE2K | 5 | 4 | K562 |
| SNHG4 | UBE4B | 5 | 1 | K562 |
| SNHG4 | UBE4B | 5 | 1 | K562 |
| SUSD1 | HSDL2 | 9 | 9 | K562 |
| SUSD1 | HSDL2 | 9 | 9 | K562 |
| SUSD1 | HSDL2 | 9 | 9 | K562 |
| SUSD1 | KIAA0368 | 9 | 9 | K562 |
| SUSD1 | KIAA0368 | 9 | 9 | K562 |
| SUSD1 | ROD1 | 9 | 9 | K562 |
| SUSD1 | ROD1 | 9 | 9 | K562 |
| TAF1 | HP1BP3 | 11 | 1 | K562 |
| TAF1 | HP1BP3 | 11 | 1 | K562 |
| TAF1 | PICALM | 11 | 11 | K562 |
| TAF1 | PICALM | 11 | 11 | K562 |
| TAF1 | PRPSAP2 | 11 | 17 | K562 |
| TAF1 | PSMA1 | 11 | 11 | K562 |
| TAF1 | PSMA1 | 11 | 11 | K562 |
| CPSF6 | BAGE3 | 12 | 21 | MCF7 |
| CPSF6 | BAGE3 | 12 | 21 | MCF7 |
| CPSF6 | C14orf135 | 12 | 14 | MCF7 |
| CPSF6 | CCT2 | 12 | 12 | MCF7 |
| CPSF6 | CNOT2 | 12 | 12 | MCF7 |
| CPSF6 | CSNK1D | 12 | 17 | MCF7 |
| CPSF6 | HNRPDL | 12 | 4 | MCF7 |
| CPSF6 | IVNS1ABP | 12 | 1 | MCF7 |
| CPSF6 | LYZ | 12 | 12 | MCF7 |
| CPSF6 | LYZ | 12 | 12 | MCF7 |
| CPSF6 | LYZ | 12 | 12 | MCF7 |
| CPSF6 | MDM2 | 12 | 12 | MCF7 |
| CPSF6 | NUP107 | 12 | 12 | MCF7 |
| CPSF6 | PGBD2 | 12 | 1 | MCF7 |
| CPSF6 | RPL3 | 12 | 22 | MCF7 |
| CPSF6 | RPL30 | 12 | 8 | MCF7 |
| CPSF6 | RPL30 | 12 | 8 | MCF7 |
| CPSF6 | SPG7 | 12 | 16 | MCF7 |
| NCOA3 | BCAS3 | 20 | 17 | MCF7 |
| NCOA3 | BCAS3 | 20 | 17 | MCF7 |
| NCOA3 | GNAS | 20 | 20 | MCF7 |
| NCOA3 | H3F3A | 20 | 1 | MCF7 |
| NCOA3 | H3F3A | 20 | 1 | MCF7 |
| NCOA3 | NPL | 20 | 1 | MCF7 |
| NCOA3 | TRIM33 | 20 | 1 | MCF7 |
| NOC4L | CNIH4 | 12 | 1 | MCF7 |
| NOC4L | EEF1D | 12 | 8 | MCF7 |
| NOC4L | FBRSL1 | 12 | 12 | MCF7 |
| NOC4L | FBRSL1 | 12 | 12 | MCF7 |
| NOC4L | FBRSL1 | 12 | 12 | MCF7 |
| NOC4L | PTDSS2 | 12 | 11 | MCF7 |
| NOC4L | PTDSS2 | 12 | 11 | MCF7 |
| NOC4L | PXMP2 | 12 | 12 | MCF7 |
| NOC4L | TMEM8A | 12 | 16 | MCF7 |
| NOC4L | ULK1 | 12 | 12 | MCF7 |
| SNHG3 | C2orf24 | 1 | 2 | MCF7 |
| SNHG3 | CCT3 | 1 | 1 | MCF7 |
| SNHG3 | CHAF1A | 1 | 19 | MCF7 |
| SNHG3 | CRAMP1L | 1 | 16 | MCF7 |
| SNHG3 | CRAMP1L | 1 | 16 | MCF7 |
| SNHG3 | DNAJC11 | 1 | 1 | MCF7 |
| SNHG3 | GGPS1 | 1 | 1 | MCF7 |
| SNHG3 | GNAS | 1 | 20 | MCF7 |
| SNHG3 | GTPBP4 | 1 | 10 | MCF7 |
| SNHG3 | LOC641298 | 1 | 16 | MCF7 |
| SNHG3 | MFF | 1 | 2 | MCF7 |
| SNHG3 | MYBL2 | 1 | 20 | MCF7 |
| SNHG3 | NDUFS1 | 1 | 2 | MCF7 |
| SNHG3 | PDS5A | 1 | 4 | MCF7 |
| SNHG3 | PDS5A | 1 | 4 | MCF7 |
| SNHG3 | PRPF3 | 1 | 1 | MCF7 |
| SNHG3 | PRPF6 | 1 | 20 | MCF7 |
| SNHG3 | PSMB2 | 1 | 1 | MCF7 |
| SNHG3 | QRICH2 | 1 | 17 | MCF7 |
| SNHG3 | RNASEH1 | 1 | 2 | MCF7 |
| SNHG3 | SERINC2 | 1 | 1 | MCF7 |
| SNHG3 | SLC38A10 | 1 | 17 | MCF7 |
| SNHG3 | SYAP1 | 1 | 23 | MCF7 |
| SNHG3 | SYAP1 | 1 | 23 | MCF7 |
| SNHG3 | TCF25 | 1 | 16 | MCF7 |
| SNHG3 | TRNAU1AP | 1 | 1 | MCF7 |
| SNHG3 | U2AF1 | 1 | 21 | MCF7 |
| SNHG3 | UIMC1 | 1 | 5 | MCF7 |
| SNHG3 | YIPF1 | 1 | 1 | MCF7 |
| TANC2 | CA4 | 17 | 17 | MCF7 |
| TANC2 | CA4 | 17 | 17 | MCF7 |
| TANC2 | CA4 | 17 | 17 | MCF7 |
| TANC2 | CA4 | 17 | 17 | MCF7 |
| TANC2 | CA4 | 17 | 17 | MCF7 |
| TANC2 | CA4 | 17 | 17 | MCF7 |
| TANC2 | FAF1 | 17 | 1 | MCF7 |
| TANC2 | GNAI3 | 17 | 1 | MCF7 |
| TANC2 | MRC2 | 17 | 17 | MCF7 |
| TANC2 | MRC2 | 17 | 17 | MCF7 |
| TANC2 | MRC2 | 17 | 17 | MCF7 |
| TANC2 | PVT1 | 17 | 8 | MCF7 |
| SNHG3 | AKR1A1 | 1 | 1 | SJCRH30 |
| SNHG3 | CCDC18 | 1 | 1 | SJCRH30 |
| SNHG3 | GNB2L1 | 1 | 5 | SJCRH30 |
| SNHG3 | KIF1B | 1 | 1 | SJCRH30 |
| SNHG3 | MORF4L2 | 1 | 23 | SJCRH30 |
| SNHG3 | MTOR | 1 | 1 | SJCRH30 |
| SNHG3 | NDUFAF4 | 1 | 6 | SJCRH30 |
| SNHG3 | OSBPL2 | 1 | 20 | SJCRH30 |
| SNHG3 | RPL5 | 1 | 1 | SJCRH30 |
| SNHG3 | SMARCC1 | 1 | 3 | SJCRH30 |
| SNHG3 | ZFR | 1 | 5 | SJCRH30 |
| LOC375010 | DCUN1D4 | 1 | 4 | SK-N-SH |
| LOC375010 | DCUN1D4 | 1 | 4 | SK-N-SH |
| LOC375010 | GOLGA8B | 1 | 15 | SK-N-SH |
| LOC375010 | PIK3C3 | 1 | 18 | SK-N-SH |
| LOC375010 | PVT1 | 1 | 8 | SK-N-SH |
| LOC375010 | ZFR | 1 | 5 | SK-N-SH |
| PPP1R12C | AKT2 | 19 | 19 | SK-N-SH |
| PPP1R12C | C19orf6 | 19 | 19 | SK-N-SH |
| PPP1R12C | CIRBP | 19 | 19 | SK-N-SH |
| PPP1R12C | FKBP8 | 19 | 19 | SK-N-SH |
| PPP1R12C | GPC1 | 19 | 2 | SK-N-SH |
| PPP1R12C | HMGA2 | 19 | 12 | SK-N-SH |
| PPP1R12C | PNCK | 19 | 23 | SK-N-SH |
| PRKAR1B | FAM20C | 7 | 7 | SK-N-SH |
| PRKAR1B | MAFK | 7 | 7 | SK-N-SH |
| PRKAR1B | PDGFA | 7 | 7 | SK-N-SH |
| PRKAR1B | SUN1 | 7 | 7 | SK-N-SH |
| PRKAR1B | SUN1 | 7 | 7 | SK-N-SH |
| SNHG3 | ATP6V1G2 | 1 | 6 | SK-N-SH |
| SNHG3 | C11orf73 | 1 | 11 | SK-N-SH |
| SNHG3 | CWF19L1 | 1 | 10 | SK-N-SH |
| SNHG3 | DCI | 1 | 16 | SK-N-SH |
| SNHG3 | FSD1 | 1 | 19 | SK-N-SH |
| SNHG3 | HNRNPC | 1 | 14 | SK-N-SH |
| SNHG3 | NMNAT1 | 1 | 1 | SK-N-SH |
| SNHG3 | PDS5A | 1 | 4 | SK-N-SH |
| SNHG3 | RPLP0 | 1 | 12 | SK-N-SH |
| SNHG3 | SENP3 | 1 | 17 | SK-N-SH |
| SNHG3 | STIP1 | 1 | 11 | SK-N-SH |
| SNHG3 | TRNAU1AP | 1 | 1 | SK-N-SH |

We have discovered a total of 98 such natural networks in 14 different cell lines (FIG. 21e). Table 9 has shown that there are 40 5′ natural networks in 10 cancer cell lines. And Table 8 has shown 58 3′ natural networks in 11 cancer cell lines. From Tables 8 and 9, we have observed that seven cell lines have both 5′ and 3′ networks. K562 cells have the most networks among the cancer cell lines, which have 30 such networks (10 5′ natural networks and 20 3′ natural networks) and count for 30% of the total identified natural networks. There is no doubt that K562 large RNA-seq datasets have contributed such abundant networks. However, the dataset sizes are not the dominate factor of identification of natural networks since both MCF-7 and SK-N-SH have much larger sequence datasets than K562 one (FIG. 4a). They have only 9 and 7 such natural networks, respectively. This suggests that such natural networks are characteristics of cancer types.

As shown in Tables 8 and 9, we have compared 5′ and 3′ natural networks. Tables 8 and 9 have shown that there are significant differences between the 5′ and 3′ natural networks. These differences suggest that these natural networks of fusion transcripts may play very roles in cellular functions.

Table 8 has shown that the 3′ most abundant network is involved with GNAS, which has highly complex imprinted expression pattern for guanine nucleotide regulatory protein and has been found to be associated with progressive osseous heteroplasia, and gnas hyperfunction. The GNAS natural networks have been found in 9 out of 11 cell lines.

Table 9 has shown that the most abundant 5′ network is the one generated by SNHG3 and has been found in 9 out of 10 cancer cell lines. It is not surprising that Table 9 has shown that many genes for non-coding RNAs such as MIR17HG, DANCR (KIAA0114) and MCM3APAS have formed networks with other genes. The natural networks formed by non-coding RNAs have raised very possibilities that observed functions of many non-coding RNAs, such as mirRNAs (Ameres and Zamore 2013), are not functions of a single MIR gene, but the network formed by a non-coding RNA gene in certain cell types under certain different environments.

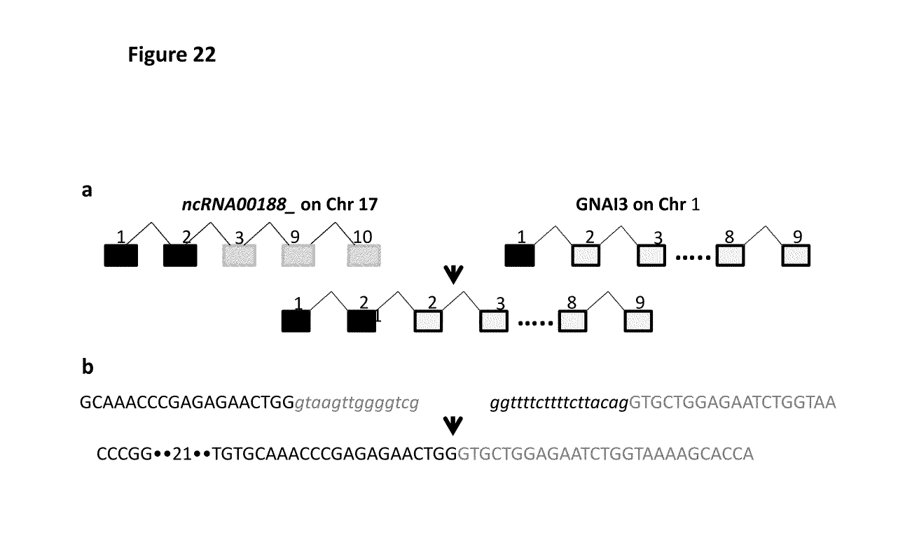

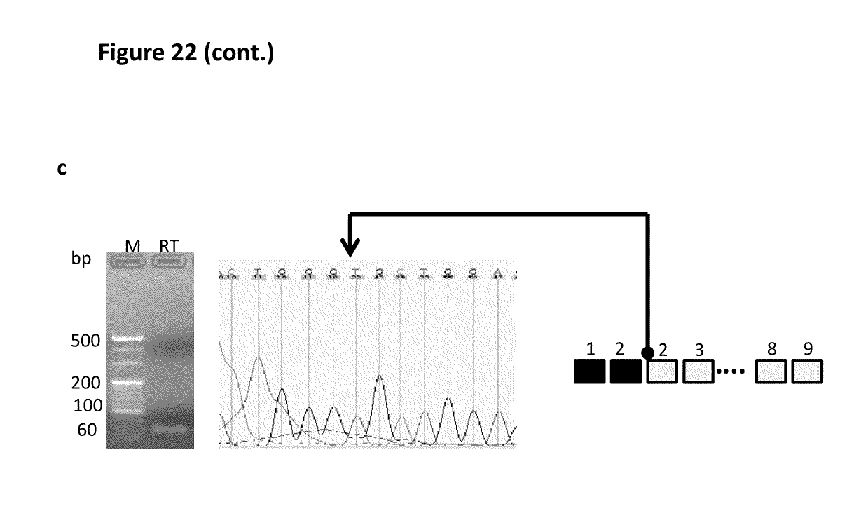

As seen from above discussions, we have proposed that none-coding RNAs have organized networks to regulate large numbers of genes and to have more powerful roles in regulating multiple cellular functions in cell lines. We have selected the some non-coding RNA fusion transcripts for validations, one of which has been validated as shown in Table 4. FIG. 22 has shown a schematic presentation of procedures to verify ncRNA00188|GNAI3 fusion transcripts. ncRNA00188 is non-coding RNA gene and is affiliated with the antisense RNA class. It has been known that GNAI3 gene coding for guanine nucleotide binding protein alpha inhibiting activity polypeptide 3, is associated with autosomal dominant Auriculocondylar syndrome (ARCND) and plays significant role in regulating downstream targets of the G protein-coupled endothelin receptor pathway (Oldham, et al. 2006). As shown in FIG. 22, ncRNA00188|GNAI3 fusion transcripts have first been detected in lymphoblastoid cells GM12878. FIG. 22a shows that ncRNA00188 gene on the chromosome 17 and GNAI3 gene on the chromosome 1 have been brought together via translocation. Solid angle lines and dashed dots represent introns and gaps, respectively. Since read-though allows the generating fusion transcripts, it is not necessary that fusion genes may not have to be truncated and may be just close to each other. The total RNAs have been isolated from lymphoblastoid cells GM12878. FIG. 22b has shown that junction sequences of ncRNA00188 and GNAI3 fusion junctions. Pre-mRNA splicing removes putative intron sequences to generate ncRNA00188|GNAI3 fusion transcripts. The primers based on fusion transcripts have been designed to amply ncRNA00188|GNAI3 cDNAs. FIG. 22c shows that the ncRNA00188|GNAI3 fusion transcript is amplified by RT-PCR. cDNA fragments are then cloned into pCR4-TOPO clone vector. The positive clones are sequenced. The fusion transcripts are verified by blast and visual inspections. FIG. 22c has shown the splice junctions of ncRNA00188|GNAI3 fusion transcripts. Arrow indicates splice junction sequences of the ncRNA00188|GNAI3 fusion transcripts. This has confirmed that the lymphoblastoid cells express non-coding RNA ncRNA00188|GNAI3 fusion transcripts. More systematic researches are required in the future to understand how these non-coding RNA fusion transcripts are regulated and expressed and to elucidate how these non-coding RNA fusion transcripts constitute natural networks to control and regulate the cell functions and how these natural networks transform the normal cells into cancer cells.

### REFERENCES CITED

Mitelman F, Johansson B, Mertens F. 2015. Mitelman Database of Chromosome Aberrations and Gene Fusions in Cancer (2015). http://cgap.nci.nih.gov/Chromosomes/Mitelman.
Klijn C, Durinck S, Stawiski E W, Haverty P M, Jiang Z, Liu H, Degenhardt J, Mayba O, Gnad F, Liu J, Pau G, Reeder J, Cao Y, Mukhyala K, Selvaraj S K, Yu M, Zynda G J, Brauer M J, Wu T D, Gentleman R C, Manning G, Yauch R L, Bourgon R, Stokoe D, Modrusan Z, Neve R M, de Sauvage F J, Settleman J, Seshagiri S, Zhang Z. 2015. A comprehensive transcriptional portrait of human cancer cell lines. Nat Biotechnol 33: 306-312.
Robinson D R, Kalyana-Sundaram S, Wu Y M, Shankar S, Cao X, Ateeq B, Asangani I A, Iyer M, Maher C A, Grasso C S, Lonigro R J, Quist M, Siddiqui J, Mehra R, Jing X, Giordano T J, Sabel M S, Kleer C G, Palanisamy N, Natrajan R, Lambros M B, Reis-Filho J S, Kumar-Sinha C, Chinnaiyan A M. 2011. Functionally recurrent rearrangements of the MAST kinase and Notch gene families in breast cancer. Nat Med 17: 1646-1651.
Sakarya O, Breu H, Radovich M, Chen Y, Wang Y N, Barbacioru C, Utiramerur S, Whitley P P, Brockman J P, Vatta P, Zhang Z, Popescu L, Muller M W, Kudlingar V, Garg N, Li C Y, Kong B S, Bodeau J P, Nutter R C, Gu J, Bramlett K S, Ichikawa J K, Hyland F C, Siddiqui A S. 2012. RNA-Seq mapping and detection of gene fusions with a suffix array algorithm. PLoS Comput Biol 8: e1002464.
Maher C A, Kumar-Sinha C, Cao X, Kalyana-Sundaram S, Han B, Jing X, Sam L, Barrette T, Palanisamy N, Chinnaiyan A M. 2009. Transcriptome sequencing to detect gene fusions in cancer. Nature 458: 97-101.
Zhao Q, Caballero O L, Levy S, Stevenson B J, Iseli C, de Souza S J, Galante P A, Busam D, Leversha M A, Chadalavada K, Rogers Y H, Venter J C, Simpson A J, Strausberg R L. 2009. Transcriptome-guided characterization of genomic rearrangements in a breast cancer cell line. Proc Natl Acad Sci USA 106: 1886-1891.
Maher C A, Palanisamy N, Brenner J C, Cao X, Kalyana-Sundaram S, Luo S, Khrebtukova I, Barrette T R, Grasso C, Yu J, Lonigro R J, Schroth G, Kumar-Sinha C, Chinnaiyan A M. 2009. Chimeric transcript discovery by paired-end transcriptome sequencing. Proc Natl Acad Sci USA.
Edgren H, Murumagi A, Kangaspeska S, Nicorici D, Hongisto V, Kleivi K, Rye I H, Nyberg S, Wolf M, Borresen-Dale A L, Kallioniemi O. 2011. Identification of fusion genes in breast cancer by paired-end RNA-sequencing. Genome Biol 12: R6.
Kim D, Salzberg S L. 2011. TopHat-Fusion: an algorithm for discovery of novel fusion transcripts. Genome Biol 12: R72.
Varley K E, Gertz J, Roberts B S, Davis N S, Bowling K M, Kirby M K, Nesmith A S, Oliver P G, Grizzle W E, Forero A, Buchsbaum D J, LoBuglio A F, Myers R M. 2014. Recurrent read-through fusion transcripts in breast cancer. Breast Cancer Res Treat 146: 287-297.
ENCODE. 2015. http://encodeprojectorg/.
ENA. 2014. http://www.ebi.ac.uk/ena.
NCBI. 2014. http://www.ncbi.nlm.nih.gov/.
Zhuo D, Madden R, Elela S A, Chabot B. 2007. Modern origin of numerous alternatively spliced human introns from tandem arrays. Proc Natl Acad Sci USA 104: 882-886.
Zhuo D, Cao W, Zhu S, Dong C, Glass ADM. 2012. Deciphoring Splicing Codes of Spliceosomal Intron. Int Conf BioInformatocs and Computational Biology 1: 521-527.
ACEVIEW. 2010. http://www.ncbi.nlm.nih.gov/IEB/Research/Acembly/Download/Downloads.html. UCSC. 2014. http://hgdownload.soe.ucsc.edu/downloads.html.
Thierry-Mieg D, Thierry-Mieg J. 2006. AceView: a comprehensive cDNA-supported gene and transcripts annotation. Genome Biol 7 Suppl 1: S12 11-14.
An integrated encyclopedia of DNA elements in the human genome. Nature 489: 57-74.
SCILIFELAB. 2015. http://www.scilifelab.se/
Yoshihara K, Wang Q, Torres-Garcia W, Zheng S, Vegesna R, Kim H, Verhaak R G. 2014. The landscape and therapeutic relevance of cancer-associated transcript fusions. Oncogene.
Asmann Y W, Necela B M, Kalari K R, Hossain A, Baker T R, Carr J M, Davis C, Getz J E, Hostetter G, Li X, McLaughlin S A, Radisky D C, Schroth G P, Cunliffe H E, Perez E A, Thompson E A. 2012. Detection of redundant fusion transcripts as biomarkers or disease-specific therapeutic targets in breast cancer. Cancer Res 72: 1921-1928.
Giacomini C P, Sun S, Varma S, Shain A H, Giacomini M M, Balagtas J, Sweeney R T, Lai E, Del Vecchio C A, Forster A D, Clarke N, Montgomery K D, Zhu S, Wong A J, van de Rijn M, West R B, Pollack J R. Breakpoint analysis of transcriptional and genomic profiles uncovers novel gene fusions spanning multiple human cancer types. PLoS Genet 9: e1003464.
Giacomini C P, Sun S, Varma S, Shain A H, Giacomini M M, Balagtas J, Sweeney R T, Lai E, Del Vecchio C A, Forster A D, Clarke N, Montgomery K D, Zhu S, Wong A J, van de Rijn M, West R B, Pollack J R. 2013. Breakpoint analysis of transcriptional and genomic profiles uncovers novel gene fusions spanning multiple human cancer types. PLoS Genet 9: e1003464.
Mercer T R, Clark M B, Crawford J, Brunck M E, Gerhardt D J, Taft R J, Nielsen L K, Dinger M E, Mattick J S. 2014. Targeted sequencing for gene discovery and quantification using RNA CaptureSeq. Nat Protoc 9: 989-1009.
SDG. 2015. http://www.yeastgenome.org
Porrua O, Libri D. 2015. Transcription termination and the control of the transcriptome: why, where and how to stop. Nat Rev Mol Cell Biol 16: 190-202.
ERP010142 SI. 2015. http://www.ebi.ac.uk/ena/data/view/ERP010142.
Kinsella M, Harismendy O, Nakano M, Frazer K A, Bafna V. 2011. Sensitive gene fusion detection using ambiguously mapping RNA-Seq read pairs. Bioinformatics 27: 1068-1075.
Koolen D A, Vissers L E, Pfundt R, de Leeuw N, Knight S J, Regan R, Kooy R F, Reyniers E, Romano C, Fichera M, Schinzel A, Baumer A, Anderlid B M, Schoumans J, Knoers N V, van Kessel A G, Sistermans E A, Veltman J A, Brunner H G, de Vries B B. 2006. A new chromosome 17q21.31 microdeletion syndrome associated with a common inversion polymorphism. Nat Genet 38: 999-1001.
de Jong S, Chepelev I, Janson E, Strengman E, van den Berg L H, Veldink J H, Ophoff R A. 2012. Common inversion polymorphism at 17q21.31 affects expression of multiple genes in tissue-specific manner. BMC Genomics 13: 458.
Stefansson H, Helgason A, Thorleifsson G, Steinthorsdottir V, Masson G, Barnard J, Baker A, Jonasdottir A, Ingason A, Gudnadottir V G, Desnica N, Hicks A, Gylfason A, Gudbjartsson D F, Jonsdottir G M, Sainz J, Agnarsson K, Birgisdottir B, Ghosh S, Olafsdottir A, Cazier J B, Kristjansson K, Frigge M L, Thorgeirsson T E, Gulcher J R, Kong A, Stefansson K. 2005. A common inversion under selection in Europeans. Nat Genet 37: 129-137.
Varley K E, Gertz J, Roberts B S, Davis N S, Bowling K M, Kirby M K, Nesmith A S, Oliver P G, Grizzle W E, Forero A, Buchsbaum D J, LoBuglio A F, Myers R M. Recurrent read-through fusion transcripts in breast cancer. Breast Cancer Res Treat 146: 287-297.
Rao P N, Li W, Vissers L E, Veltman J A, Ophoff R A. 2010. Recurrent inversion events at 17q21.31 microdeletion locus are linked to the MAPT H2 haplotype. Cytogenet Genome Res 129: 275-279.
Charlier C, Segers K, Wagenaar D, Karim L, Berghmans S, Jaillon O, Shay T, Weissenbach J, Cockett N, Gyapay G, Georges M. 2001. Human-ovine comparative sequencing of a 250-kb imprinted domain encompassing the callipyge (clpg) locus and identification of six imprinted transcripts: DLK1, DAT, GTL2, PEG11, antiPEG11, and MEG8. Genome Res 11: 850-862.
Gutschner T, Diederichs S. 2012. The hallmarks of cancer: a long non-coding RNA point of view. RNA Biol 9: 703-719.
Williams G T, Mourtada-Maarabouni M, Farzaneh F. 2011. A critical role for non-coding RNA GASS in growth arrest and rapamycin inhibition in human T-lymphocytes. Biochem Soc Trans 39: 482-486.
Tripathi V, Ellis J D, Shen Z, Song D Y, Pan Q, Watt A T, Freier S M, Bennett C F, Sharma A, Bubulya P A, Blencowe B J, Prasanth S G, Prasanth K V. 2010. The nuclear-retained noncoding RNA MALAT1 regulates alternative splicing by modulating S R splicing factor phosphorylation. Mol Cell 39: 925-938.
Ghoussaini M, Song H, Koessler T, Al Olama A A, Kote-Jarai Z, Driver K E, Pooley K A, Ramus S J, Kjaer S K, Hogdall E, DiCioccio R A, Whittemore A S, Gayther S A, Giles G G, Guy M, Edwards S M, Morrison J, Donovan J L, Hamdy F C, Dearnaley D P, Ardern-Jones A T, Hall A L, O'Brien L T, Gehr-Swain B N, Wilkinson R A, Brown P M, Hopper J L, Neal D E, Pharoah P D, Ponder B A, Eeles R A, Easton D F, Dunning A M. 2008. Multiple loci with different cancer specificities within the 8q24 gene desert. J Natl Cancer Inst 100: 962-966.
Enwerem, I I, Velma V, Broome H J, Kuna M, Begum R A, Hebert M D. 2014. Coilin association with Box C/D scaRNA suggests a direct role for the Cajal body marker protein in scaRNP biogenesis. Biol Open 3: 240-249.
An S, Song J J. 2011. The coded functions of noncoding RNAs for gene regulation. Mol Cells 31: 491-496.
Olive V, Jiang I, He L. 2010. mir-17-92, a cluster of miRNAs in the midst of the cancer network. Int J Biochem Cell Biol 42: 1348-1354.
Olive V, Li Q, He L. 2013. mir-17-92: a polycistronic oncomir with pleiotropic functions. Immunol Rev 253: 158-166.
Penna E, Orso F, Taverna D. 2015. miR-214 as a key hub that controls cancer networks: small player, multiple functions. J Invest Dermatol 135: 960-969.
Pelczar P, Filipowicz W. 1998. The host gene for intronic U17 small nucleolar RNAs in mammals has no protein-coding potential and is a member of the 5′-terminal oligopyrimidine gene family. Mol Cell Biol 18: 4509-4518.
Guttman M, Donaghey J, Carey B W, Garber M, Grenier J K, Munson G, Young G, Lucas A B, Ach R, Bruhn L, Yang X, Amit I, Meissner A, Regev A, Rinn J L, Root D E, Lander E S. 2011. lincRNAs act in the circuitry controlling pluripotency and differentiation. Nature 477: 295-300.
Ameres S L, Zamore P D. 2013. Diversifying microRNA sequence and function. Nat Rev Mol Cell Biol 14: 475-488.
Oldham W M, Van Eps N, Preininger A M, Hubbell W L, Hamm H E. 2006. Mechanism of the receptor-catalyzed activation of heterotrimeric G proteins. Nat Struct Mol Biol 13: 772-777.

1. A method of detecting alternatively spliced transcripts or fusion transcripts in at least one RNA sequence obtained from biochemical analysis of a biological sample from a species or from a database, comprising the steps of:
(a) providing a computer for data identification, aligning, and comparison purposes, wherein the computer has access to predetermined genome data of said species, comprising data of predetermined genomic nucleotide sequences, predetermined splicing junctions, predetermined exons, predetermined introns, and annotated genes;
(b) generating a splicing code table using the predetermined genome data, the splicing code table comprising ordered E5 keys, I5 keys, E3 keys and I3 keys, wherein the E5 keys, the I5 keys, the E3 keys and the I3 keys are subsequences of predetermined 5′ exonic (E5), 5′ intronic (I5), 3′ exonic (E3), and 3′ intronic (I3) splicing sequences for each of the predetermined splicing junctions respectively;
(c) aligning the at least one RNA sequence with each of the E5 keys and each of the E3 keys in the splicing code table; and
(d) determining that the at least one RNA sequence is an alternatively spliced transcriptif:
the at least one RNA sequence contains a first subsequence substantially identical to an E5 key of a first splicing junction and a second subsequence substantially identical to an E3 key of a second splicing junction of the same gene; or
the at least RNA sequence contains a subsequence substantially identical to an E5 key of an annotated gene, but an immediate downstream sequence of said subsequence is mapped to an intron region of the same annotated gene; or
the at least one RNA sequence contains a subsequence substantially identical to an E3 key of a splicing junction, but an immediate upstream sequence of said subsequence is mapped to an intron region of the same annotated gene;
or determining that the at least one RNA sequence is a fusion transcriptif:
the at least one RNA sequence contains a subsequence substantially identical to an E5 key of a first annotated gene, and an immediate downstream sequence of said subsequence is substantially identical to an E3 key of a second annotated gene; or
the at least RNA sequence contains a subsequence substantially identical to an E5 key of a first annotated gene, and an immediate downstream sequence of said subsequence is mapped to a second annotated gene; or
the at least one RNA sequence contains a subsequence substantially identical to an E3 key of a first annotated gene, and an immediate upstream sequence of said subsequence is mapped to a second annotated gene.

2. The method of claim 1, wherein the E5 keys, the I5 keys, the E3 keys and the I3 keys in the splicing code table in step (b) have a length of about 20-50 bp.

3. The method of claim 1, wherein the at least one RNA sequence is obtained from RNA sequencing.

4. The method of claim 1, wherein the at least one RNA sequence is obtained from a biochemical analysis comprising RT-PCR.

5. The method of claim 1, wherein the at least one RNA sequence is obtained from a database.

6. The method of claim 1, further comprising a quality control step between step (b) and step c), wherein the quality control step comprises removing reads from the at least one RNA sequence, wherein the reads have substantially same sequences as at least one of mitochondrial gene sequences, mitochondrial ribosomal RNA sequences, ribosomal RNA sequences, poly (A) sequences, GC-repetitive sequences, AT-rich sequences, and simple and contaminant sequence reads.

7. The method of claim 1, wherein the species is an eukaryotic organism.

8. The method of claim 7, wherein the species is a mammal.

9. The method of claim 8, wherein the species is human.

10. A method of characterizing at least one RNA sequence read in a transcriptome dataset, obtained from a transcriptome sequencing of a biological sample, for fusion transcripts, the method comprising the steps of:
(a) providing a computer for data identification, aligning, comparison and computation purposes, wherein:
the computer has access to the transcriptome dataset, the transcriptome dataset comprising data of genome-wide RNA sequence reads and counts thereof and; and
the computer has access to a predetermined fusion transcript table, the predetermined fusion transcript table comprising data of predetermined E5-E3 keys, wherein:
each of the predetermined E5-E3 keys corresponds to junction sequence of a predetermined fusion transcript, comprising an E5 key and an E3 key, wherein:
the E5 key corresponds to a 5′-end subsequence of the predetermined fusion transcript and is mapped to a first annotated gene;
the E3 key corresponds to a 3′-end subsequence of the predetermined fusion transcript and is mapped to a second annotated gene; and
the E5 key and the E3 key is connected at a junction of the predetermined fusion transcript;
(b) aligning the at least one RNA sequence read with each of the E5-E3 keys in the predetermined fusion transcript table;
(c) determining that the at least one RNA sequence read is mapped to a predetermined fusion transcript if the at least one RNA sequence read contains a subsequence substantially identical to an E5-E3 key in the predetermined fusion transcript table.

11. The method according to claim 10, further comprising, following step (c), a step of determining expression level of the predetermined fusion transcript to which the at least one RNA sequence read is mapped in the biological sample, the step comprising:
(i) determining that E5 key and E3 key of the E5-E3 key, which corresponds to the predetermined fusion transcript, are unique in the transcriptome dataset; and
(ii) determining the expression level of the predetermined fusion transcription the biological sample, by dividing the count of the at least one RNA sequence read by sum of the counts of the genome-wide RNA sequence reads in the transcriptome dataset.

12. A set of isolated, cloned recombinant or synthetic polynucleotides, comprising at least one polynucleotide, wherein:
each of the at least one polynucleotide encodes a fusion transcript, the fusion transcript comprising a 5′ portion from a first gene and a 3′ portion from a second gene, wherein:
the 5′ portion from the first gene and the 3′ portion from the second gene is connected at a junction;
the junction has a flanking sequence, comprising a sequence selected from the group of nucleotide sequences as set forth in SEQ ID NOs: 1-258,853, or from complementary sequences thereof.

13. The set of polynucleotides according to claim 12, wherein the junction has a flanking sequence selected from the group of nucleotide sequences as set forth in SEQ ID NOs: 1-258,077.

14. A composition for detecting, from a biological sample from a subject, the set of polynucleotides as set forth in claim 12, comprising at least one of the following:
(a) at least one probe, wherein each of the at least one probe comprises a sequence that hybridizes specifically to a junction of a fusion transcript encoded by one of the set of polynucleotides;
(b) at least one pair of probes, wherein each of the at least one pair of probes comprises:
a first probe comprising a sequence that hybridizes specifically to a first gene of a fusion transcript encoded by one of the set of polynucleotides; and
a second probe comprising a sequence that hybridizes specifically to a second gene of the fusion transcript; or
(c) at least one pair of amplification primers, wherein each of the at least one pair of amplification primers comprise:
a first amplification primer comprising a sequence that hybridizes specifically to a first gene of a fusion transcript encoded by one of the set of polynucleotides;
a second amplification primer comprising a sequence that hybridizes specifically to a second gene of the fusion transcript; and
a means for detecting an amplified product generated between the first amplification primer and the second amplification primer.

15. The composition according to claim 14, comprising in (a) a plurality of probes, and a substrate on which the plurality of probes are immobilized.

16. The composition according to claim 14, further comprising a means for generating cDNA molecules from mRNA molecules in the biological sample.

17. A method for detecting, from a biological sample from a subject, the presence of at least one of the set of polynucleotides as set forth in claim 12, comprising:
(a) performing a biochemical assay on the biological sample, using at least one gene fusion informative composition for detection of the at least one of the set of polynucleotides; and
(b) determining the presence, or absence, of the at least one of the set of polynucleotides in the biological sample.

18. The method of claim 17, wherein in step (a) the biochemical assay comprises a nucleic acid hybridization technique, selected from the group consisting of: in situ hybridization (ISH), microarray analysis, and Northern blot analysis.

19. The method of claim 18, wherein the nucleic acid hybridization technique is microarray analysis, comprising the sub-steps of:
(i) isolating mRNA molecules from the biological sample;
(ii) converting the mRNA molecules into cDNA molecules, and optionally amplifying the cDNA molecules;
(iii) labeling the cDNA molecules;
(iv) hybridizing the labeled cDNA molecules to a microarray chip, wherein:
the microarray chip comprises a plurality of probes and a substrate;
the plurality of probes are immobilized on the substrate; and
each of the plurality of probes comprises an oligonucleotide sequence that hybridizes specifically to a junction of a fusion transcript encoded by one of the set of polynucleotides; and
(v) detecting a pattern of hybridization for each of the plurality of probes.

20. The method of claim 17, wherein in step (a) the biochemical assay comprises a nucleic acid amplification technique, selected from the group consisting of: polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA).

21. The method of claim 20, wherein the nucleic acid amplification technique is reverse transcription polymerase chain reaction (RT-PCR), comprising the sub-steps of:
(i) isolating mRNA molecules from the biological sample;
(ii) converting the mRNA molecules into cDNA molecules;
(iii) performing at least one PCR on the cDNA molecules, using at least one pair of amplification primers, wherein each of the at least one pair of amplification primers comprise:
a first amplification primer comprising a sequence that hybridizes specifically to a first gene of a fusion transcript encoded by one of the set of polynucleotides;
a second amplification primer comprising a sequence that hybridizes specifically to a second gene of said fusion transcript encoded by one of the set of polynucleotides; and
(iv) detecting amplification products from the at least one PCR.
